(19) United States
(12) Patent Application Publication
(10) Pub. No.: US 2024/0130747 A1
(43) Pub. Date: Apr. 25, 2024

(54) Knowledge-Based Thrombectomy System

(71) Applicant: J. Michael Shifflette, Alachua, FL (US)

(72) Inventor: J. Michael Shifflette, Alachua, FL (US)

(21) Appl. No.: 18/380,784

(22) Filed: Oct. 17, 2023

Related U.S. Application Data
(60) Provisional application No. 63/525,624, filed on Jul. 7, 2023.
(60) Provisional application No. 63/416,831, filed on Oct. 17, 2022.

(51) Int. Cl. A61B 17/22; A61B 90/00

(52) U.S. Cl. CPC A61B 17/22012; A61B 90/06; A61B 2017/00022

(57) ABSTRACT

Thrombectomy systems are disclosed that utilize a knowledge base comprising intra-procedural and inter-procedural (i.e., historical) measurement data that is correlated to deterministic events that predicate each measurement. Each thrombectomy procedure is thereby a compendium of cause and effect experimentation; experimental data are retained, in addition to being utilized intra-procedurally to determine future experimental configurations (deterministic events). Herein, experimental data comprise variable measurement data of intensive physical properties of the aspirate within the catheter (e.g., viscosity=10 cP, % thrombus=15%, thrombus load=25%, etc.). Knowledge-based thrombectomy systems engender procedure standardization across multiple thrombectomy systems, facilities and clinicians by standardizing the sequence of intra-procedural, deterministic events. Some embodiments feature a plurality of subsystems (experimental factors) such as Liquid Column Oscillator, Harmonic Oscillator, frequency, aspiration rate, infusion rate, mechanical or hydrodynamic lance, catheter position and/or configuration, etc.; these subsystems being operable at a plurality of setpoints (experimental levels). A knowledge base is compiled that identifies and exploits efficacious experimental configurations and abandons or modifies inefficacious experimental configurations.

### STATEMENT OF RELATED CASES

This case relates to U.S. patent application Ser. No. 17/409,635 filed Aug. 23, 2021, Ser. No. 18/128,224 filed Mar. 30, 2023, and Provisional U.S. Pat. App. 63/525,624 filed Jul. 7, 2023 and 63/416,831 filed on Oct. 17, 2022, any or all of which are incorporated by reference herein.

### FIELD OF THE INVENTION

The present invention relates to thrombectomy, control systems, oscillatory flow, resonance, flow measurement and viscometry.

### BACKGROUND INFORMATION

Thrombectomy systems and devices are intended to remove occlusive deposits (thrombus) from the interior walls of the vascular system by means a long tube or catheter. Some thrombectomy systems employ extracorporeal vacuum which evacuates (aspirates) blood and thrombus through a catheter or hollow tube. Other thrombectomy systems employ manually manipulated obturators and/or rotating cutters to mechanically macerate thrombus, which may be aspirated by vacuum. Other thrombectomy systems employ hydrodynamic (or rheolytic) effects, where liquid is infused into the catheter; this liquid may be delivered at high pressures (e.g., up to approximately 10,000 psi, 666 bar) such that a high velocity liquid jet performs maceration of thrombus. Some thrombectomy systems deliver hemolytic or thrombolytic agents to chemically decompose solid thrombus to a liquid or less-viscous state.

The composition and physical properties of thrombus vary widely from soft, gelatinous material such as collagen, to hard, fibrotic masses with strong adhesion to the vessel wall. The physical properties range from elastic to plastic and localized discontinuities may exist wherein a network or matrix of plastic material is interspersed with elastic material. Softer thrombus may be successfully aspirated with a vacuum system, while harder thrombus may require mechanical maceration as may be provided by mechanical or hydrodynamic maceration.

Mechanical (e.g., obturator, rotating cutter, etc.) and hydrodynamic (e.g., liquid jet, ultrasonic, etc.) thrombectomy systems should be designed so as not to injure the vasculature while macerating and aspirating thrombus. Thrombectomy systems should be designed to minimize the loss of viable blood. Hemolytic thrombectomy systems pose risks to both blood and the vascular system because the hemolytic agents decompose viable blood and vascular tissue ad well as the targeted thrombus. Once infused into thrombus and the vascular system, the hemolytic agents are not confined to the location of the thrombus; hemolysis of viable blood and decomposition of vascular tissue are systemic and not localized to the lesion (site of thrombus).

Some thrombectomy systems are outfitted with instrumentation such as one or

more pressure transducers, a goal being to draw inferences about the flow rate of fluid through the catheter. Inferences from pressure transducer data which are characterized in terms such as “free flow” or “open flow” are used as justification of further inference that the catheter is aspirating blood with no appreciable thrombus present. Inferences from pressure transducer data which are characterized in terms such as “clot” or “clog,” are used as justification of further inference that at least a portion of the catheter contents is comprised of thrombus. Continued operation of a thrombectomy system under “free flow” or “open flow” conditions is disadvantageous because blood loss may be appreciable. A “clogged” or “corked” catheter may require manual clinician intervention, which can be time consuming and adversely affect the procedure efficacy.

### SUMMARY OF THE INVENTION

Embodiments of the invention provide a thrombectomy system that, in addition to other capabilities, is capable of determining and setting efficacious setpoints for system parameters in real time. Embodiments include one or more adjustable system parameters (e.g., aspiration rate, infusion rate, catheter configuration, oscillatory frequency, etc.) that may be setpoint-controlled by a system controller using closed-loop feedback of viscometric measurement data obtained by the system. In some embodiments, a sequential execution of a plurality of thrombectomy operating modes is correlated to viscometric measurement data to quantitatively determine thrombectomy efficacy.

The various embodiments of invention differ from the prior art in many ways, a few of which are described below; to better understand those differences, it is illustrative to first consider the apparatus and control strategies of prior art.

Thrombectomy systems of prior art feature an aspiration catheter and instrumentation tasked to answer the question: what is the flow rate through the catheter? Measuring the flow rate in a thrombectomy catheter has proven elusive, for reasons outlined in the inventor's co-pending US Patent Application Time-Domain Viscometry, listed as a reference. The prior art, seeking and failing to measure the flow rate, utilizes an attribute data classification system to describe the flow using words such as: “characteristic of flow,” “flow state,” “free flow,” “restricted flow,” “occluded flow,” etc., wherein the flow may be monitored for change in the “characteristic of flow.” Upon detection of a change in monitored flow, prior art thrombectomy systems make changes to system setpoints (open/close a valve, hold a valve open, adjust a pump speed, adjust a pressure level, etc.) in reaction to the change. Prior art monitors flow for a change of flow state or characteristic resulting from random events occurring with the passage of time. The point in time that an uncontrolled or random event caused the flow state or characteristic to change predicates the point in time that the system changes the apparatus setpoints. Note that the designers of prior art thrombectomy chose to measure or monitor flow rate, which is an extensive property of a system (apparatus+patient) that is dependent upon factors such as differential pressure, catheter diameter/length, etc.

Among other structures and functionalities, embodiments of thrombectomy systems or apparatus of the invention also feature an aspiration catheter and instrumentation; however an entirely different control strategy is utilized. Embodiments of the invention ask entirely different questions, including: how much thrombus is currently in the catheter, what apparatus setpoints caused that amount of thrombus to be in the catheter, and what apparatus setpoints will cause an optimum amount of thrombus to be in the catheter in the future. Alternatively to measuring or monitoring flow, embodiments of the invention quantitatively measure the viscosity of the aspirate to quantitatively measure the amount of thrombus in the aspirate. Embodiments of the invention systematically adjust apparatus setpoints, and operate the apparatus for a duration sufficient to measure the viscosity of the aspirate. The apparatus thereby executes a sequence of deterministic events (or conducts successive experiments) in thrombectomy efficacy, and correlates the measured thrombus in the aspirate to the apparatus setpoints. The resulting data, correlating thrombus to apparatus setpoints, is used to alter the sequence of deterministic events (experiments) that are conducted in the future. Thrombectomy systems of prior art correlate changes in characteristic of flow to time and correlate reactionary changes in apparatus setpoints to changes in characteristic of flow; therefore changes in apparatus setpoints are correlated to the passage of time. In contrast, embodiments of the invention correlate measurements of thrombus to deterministic events that predicate the amount of thrombus in the catheter and the measurement. The inventor chose to measure viscosity, an intensive property of the fluid within the catheter. Designers of prior art thrombectomy systems chose to measure/monitor an extensive system property, flow (rate), which is continuously changing with changes in aspiration rates or differential pressure and is markedly influenced by catheter diameter and length. The inventor chose to measure an intensive property, viscosity, of the fluid within the catheter, which is independent of aspiration rates, differential pressure, catheter length/diameter or other extensive property. The inventor has chosen a more fundamental system parameter to measure, fluid viscosity being independent of other system parameters; the resulting experiment designs disclosed herein are well-devised and are not confounded by variation in other system parameters.

Embodiments of the invention invoke an intensive physical property —viscosity— that is measured, thereby quantitatively determining relevant information regarding the composition and relative flow rate of aspirate generally contained within a catheter. Examples include: viscosity, μ≈6 centiPoise (cP), relative flow rate, Qr≈67% (as compared to blood), % thrombus≈5%, where % thrombus is a quantitative measure of the amount of thrombus in the catheter. The prior art typically seeks information about an extensive physical property of the thrombectomy system: flow (rate). Prior art methods provide a qualitative characterization of attributes (e.g., “characteristic of flow,” ‘flow state,” “free flow,” “open flow,” “restricted flow,” “clot,” “clog,” etc.), which limits the usefulness of such methods.

Unlike the prior art, embodiments of the invention obtain viscometric samples of the aspirate within the catheter and thereby measure the amount of thrombus in the catheter. The sample volume may be small (c.a., 0.2 cc, range of approximately 0.0 cc<sample volume<5 cc) and rapidly obtained (c.a., 0.5 s, range of 0.2 s to 5 s). The small sample volume and rapid analysis contribute to procedural efficiency. Viscometric sampling further enables an inference of the proximity/distance between the catheter tip and a target thrombus. This enables clinicians (or automated systems) to better position the catheter prior to aspiration so that successive thrombectomy operating modes are executed with the catheter in proper proximity to a thrombus. Furthermore, if the catheter is in a sub-optimal location such as piercing or penetrating a thrombus, with an impending clog forming, automated selection of subsequent thrombectomy operating modes will reduce the likelihood of clogging or corking a catheter during aspiration. If the catheter is in a sub-optimal location, such as too far away to attrite and aspirate thrombus, manual or automated methods to advance the catheter are incorporated in some embodiments.

Some prior art thrombectomy systems employ methods that utilize experimental measurements, such as pressure, as arguments in conditional statements (e.g., IF [measured pressure<REFERENCE VALUE] THEN [open valve], etc.), wherein the REFERENCE VALUE may be a manufacturer-supplied arbitrary constant, threshold value, pre-determined value, library data, or other reference data. Since the REFERENCE VALUE has no specific connection to the system of interest, the use of such data may bias the outcome of the conditional statement. Moreover, the prior art may first assign attribute data (e.g., “characteristic of flow,” free flow, open flow, clot, clog, restricted flow, etc.) and then utilize the assigned attribute data as arguments in Boolean operations (e.g., is the current “characteristic of flow” equal to the last “characteristic of flow?”, etc.). This limits the analysis to two possible outcomes, such as “yes or no,” “true or false,” “change or no change,” etc. In contrast, embodiments of the invention utilize quantitative analysis (e.g., algebraic, ratiometric, etc.) of collected data (e.g., viscosity, pressure, etc.) with respect to similar data collected earlier in the procedure. Here, both the same apparatus and the same clinical setting are used, remaining unchanged from calibration through procedure endpoint. Consequently, conditional statements executed in the present methods utilize current, intra-procedural, inter-procedural and/or historical data as threshold values and arguments in conditional statements.

Whereas prior-art thrombectomy systems typically exert automated control over one system parameter (e.g., valve on/off, pump on/off/speed, etc.), embodiments of the invention exert simultaneous, automated control over a plurality of system parameters or factors (e.g., aspiration pump, infusion pump, Low Frequency Oscillator frequency, Harmonic Oscillator frequency, catheter geometry, etc.) at a plurality of setpoints or levels (e.g., 200 RPM, 25 Hz, 3 mm extension, etc.).

Prior-art thrombectomy systems collect data (e.g., pressure, etc.) and utilize it to characterize flow (e.g., free flow, restricted flow, open flow, clot, etc.) to determine a “next step” (e.g., open valve, turn on pump, remove catheter to clear clog, etc.). Thereafter, such collected data and inferences (e.g., “characteristic of flow,” etc.) are discarded at the conclusion of each step. Embodiments of the present invention, however, provide ongoing cause-and-effect analyses which are retained as a procedure log. Each thrombectomy operating mode that is executed is correlated to a quantitative measurement of thrombectomy efficacy. Efficacious thrombectomy operating modes may be exploited and inefficacious thrombectomy operating modes may be abandoned. A thrombectomy procedure, executed in accordance with the present teachings, typically executes thousands (c.a., range of approximately 100 to 20,000) of cause-and-effect experiments in thrombus extraction through a catheter. These data, which are collected from a single procedure, may be incorporated into a compilation of similar procedures, thereby enabling further correlations and cross-correlations with measurable statistical significance.

In the prior art, thrombectomy systems typically monitor data for changes in parameters such as pressure or flow with respect to time. Prior art thrombectomy systems may thereby only correlate monitored data for changes with the passage of time. In contrast, embodiments of the invention first invoke deterministic events and then measure the resultant aspirate viscosity; outcomes of the measurements are predicated upon deterministic events. Embodiments of the invention measure aspirate viscosity and correlate the measurement data to deterministic events, rather than time. Example deterministic events include:
thrombectomy operating mode, aspiration levels, catheter positioning, oscillatory frequencies, infusion rates, catheter geometries, etc.

In the prior art, each thrombectomy system is independently (and autonomously) operated in facilities such as hospitals, clinics, private practices by one or more clinicians. Each clinician utilizes a different skill-set acquired through experience, expertise, intuition, training, etc.; furthermore, each clinician is tasked with qualitatively determining procedure steps, endpoint determination and assessing procedure efficacy. Embodiments of the invention enable standardization of multiple procedures conducted at multiple facilities by multiple clinicians. For instance, embodiments of the invention include standardization of the distance between a catheter tip and a thrombus prior to aspiration or other thrombectomy operating mode. This standardization concomitantly limits blood loss (in cases where the distance is too large) and risk of clogging or corking a catheter (in cases where the catheter has pierced or penetrated a thrombus). Further procedure standardization is enabled by embodiments of the invention that execute a repeatable sequence of treatment regimens (thrombectomy operating modes) combined with data-driven feedback and adjustments to the sequence. Embodiments of the invention thereby enable a degree of automation to thrombectomy procedures for repeatable and reproducible results irrespective of facility or clinician. Embodiments of the invention enable novice clinicians (perhaps in poorly-quipped, remote or otherwise inauspicious facilities) to deliver patient outcomes that rival exemplary patient outcomes delivered by expert clinicians (perhaps in well-equipped facilities with extensive experience and expertise in thrombectomy).

Objectives of a thrombectomy procedure include attriting and aspirating a maximum quantity of thrombus concomitantly with a minimum quantity of blood loss in a manner that does not clog the catheter and is not injurious to vasculature or other tissues. After a device (e.g., catheter, guidewire, obturator, lance, etc.) is deployed and positioned in proper proximity to a thrombus, there exist a great many techniques (and combinations of techniques) that may be executed (e.g., maximum suction for a short period of time, reduced suction for a long period of time, pulsed suction, 500 psi infusion pressure, twist and/or push catheter or obturator, etc.). In thrombectomy systems of prior art, an experienced and/or skilled clinician may execute one or more thrombectomy techniques (i.e., treatment regimen or thrombectomy operating modes) that efficaciously aspirate thrombus, whereas other clinicians may their own training or intuition with more or less efficacious results. Some embodiments of the invention standardize the selection and sequence of thrombectomy operating modes for increased procedure uniformity and standardization.

The present invention provides solutions to several problems identified above: (1) difficulty in determining the proper proximity between the device and a thrombus, (2) inability to determine the amount of thrombus aspirated as result of each technique executed, (3) randomness in determining the proper combination and/or sequence of techniques that improve procedure efficacy and (4) a general lack of standardization between procedures. Solution of these problems, by methods of the present invention, may be enabled by invoking real-time, quantitative measurement of the aspirate viscosity concomitantly with each executed treatment regime, thrombectomy operating mode or experiment (deterministic events).

Thrombectomy procedures are typically indicated for patients that present with non-flowing masses of thrombus within the vasculature; the thrombi are typically attached to, or otherwise immobilized with respect to the vasculature. A patient that presents with thrombotic complications does so with a specific amount of thrombus burden (e.g., 100g, 125g, etc.) within the vasculature. Thrombectomy procedures do not typically extract 100% of the thrombus burden of the patient, rather certain target thrombi are identified for treatment by thrombectomy. For example, a patient may present with a total of 100 grams of thrombus irregularly distributed about the vasculature; of this, an example 20 gram portion of the total 100 grams of thrombus is problematic, typically due to anatomical considerations, e.g., blood flow through certain portions of the vasculature is reduced or occluded by a thrombus. A corresponding example thrombectomy procedure is therefore tasked with aspirating a maximum amount of the problematic 20 grams of thrombus, thereby attriting the total thrombus burden of the patient. The corresponding 100% successful thrombectomy procedure aspirates the entire 20 grams of problematic thrombus; however the remaining 80 grams of non-problematic thrombus remains within the patient vasculature. The example thrombectomy procedure thereby attrites and aspirates thrombus. During the course of a thrombectomy procedure, thrombus may be attrited by aspiration alone (as in prior art); however additional means of thrombus attrition may be utilized in conjunction with aspiration. Example additional means include: LF oscillator 102, Harmonic Oscillator 104, LCO with aspiration and infusion 400, hydrodynamic lance, mechanical lance, obturator, rotating cutters, stent retrievers, guidewires, etc. Embodiments of the invention reduce, decrease or otherwise limit aspiration setpoints (e.g., pump speed, setpoint pressure, duration of aspiration) such that potential or imminent clog occurrences are averted. Some embodiments include active measures to avert potential or imminent clogs by means such as: impulse mechanism 90, LF oscillator 102, LCO with aspiration and infusion 400; these example embodiments represent means to attrite thrombus and/or means to avert clogs.

A typical catheter is generally analogous to a long pipe, for which analytical (e.g., Poiseuille's Equation, etc.) and graphical solutions exist (e.g., Moody Chart, etc.) that relate the parameters including viscosity, differential pressure, flow etc. In a long pipe (such as an aspiration catheter), a quantitative measurement of viscosity may enable the calculation of a quantitative measurement of flow rate. However, quantification of the extensive property flow rate may be accomplished at the expense of error and computational resources; one reason is that the differential pressure between a vacuum/suction/aspiration source and the patient bloodstream is unsteady (or constantly changing); therefore the flow rate is also continuously changing and may need to be tracked and averaged throughout many pressure cycles. Conversely, the intensive property, viscosity, remains constant (independent of differential pressure and flow rate); when measured viscosity does change, embodiments of the present invention infer that the aspirate composition (e.g., % saline, % blood, % thrombus, etc.) has changed. Measurements of aspirate viscosity may be converted to engineering units (e.g., cP, cS, Pas, etc.) by straightforward in-situ calibration with reference fluids including patient blood and/or saline. Any calculated or derived quantification of aspirate composition (e.g., % saline, % blood, % thrombus, relative flow rate, thrombus load, etc.) may be obtained from viscometric measurements by algebraic or ratiometric means.

To provide relevance to prior art and qualitative flow rate characterization, calculation of relative flow rate, (e.g., Qr, as may be measured viscometrically) is included herein. A large or maximum relative flow rate (e.g., Qr>≈95%, Qr≈100%, etc.) may represent undesirable blood loss (because the catheter contains predominantly blood and not much thrombus). A small or near zero relative flow rate (e.g., Qr<10%, Qr≈0%) may be indicative of heavily thrombus-laden catheter or a clog. An intermediate relative flow rate (e.g., approximately 10%<Qr<95%, etc.) may be indicative of efficacious aspiration of thrombus because the aspirate may be inferred to comprise a blood/thrombus mixture. It may also be inferred that any increase in measured viscosity of the aspirate may be correlated to an increase in the quantity of thrombotic matter (in the aspirate) which may tend to diminish, limit, restrict or even occlude flow. The present invention comprises quantitative measurement of aspirate viscosity, which may be non-standard in the industry vernacular; subsequent calculation of derived quantities including relative flow rate (Qr) and % thrombus are included herein because (1) flow rate is an industry-standard term and (2) % thrombus is intuitively meaningful. Note, however, that flow rate (of prior art efforts) is an extensive property (expressed in units such as grams/second, cc/second, gallons/minute, etc.); in contrast, both relative flow rate (e.g., Qr) and % thrombus are derived, intensive properties (comprising the present invention) and may be expressed in units such as mass fraction, volume fraction, percentage, etc.

Prior art attempts to create thrombectomy systems that feature flow measurement have been elusive, and terms such as “flow monitoring,” and “characteristic of flow” have been used in technical literature; examples include “free flow,” “restricted flow,” “full flow,” “flow state,” “clot,” and “clog,” none of which are quantitative. The present applications intentionally select viscosity as a parameter to be measured, because viscosity is an intensive property of a fluid. In contrast, flow rate is an extensive property wherein system parameters or variables (e.g., catheter diameter and length, differential pressure, etc.) affect (e.g., scale, multiply, etc.) the measured flow rate. As an example, a large diameter catheter may exhibit an absolute flow rate for blood (μ≈4 cP) of approximately 100cc/minute, whereas a small diameter catheter may exhibit an absolute blood flow rate of only approximately 10cc/minute. But that same large catheter may exhibit an absolute flow rate for SAE 30 motor oil (μ≈40 cP) of approximately 10cc/minute and the same small diameter catheter may exhibit an absolute flow rate of only approximately 1cc/minute. The extensive property absolute flow rate may need to be scaled by system parameters (e.g., catheter diameter/length, differential pressure, etc.) whereas the intensive property viscosity (and relative flow rate and % thrombus) is independent of size or scale. Embodiments of the present invention may take initial measurements of the viscosity of blood (and optionally saline as an additional calibration point); this in-situ instrument calibration is performed upon the specific catheter (manufacturer part number, serial number, including dimensional or quality aberrations, etc.) used throughout the procedure such that scaling factors or corrections for catheters of different lengths and/or diameters are typically unnecessary. Preferred embodiments of the present invention measure aspirate viscosity and perform algebraic or ratiometric calculations thereupon; if deemed necessary, viscosity measurements may be converted into the extensive property of absolute flow rate (in units including: cc/second, grams/second, etc.).

Embodiments of the present invention utilize methods including viscometric sampling wherein a small, zero or near zero aspirate sample volume (less than approximately 5cc) is analyzed for viscosity as fully described in the references. An embodiment of a thrombectomy system suitable for viscometric sampling comprises the following components: (a) catheter, (b) peristaltic pump, (c) pressure transducer and (d) system controller. An example method of viscometric sampling is as follows: (i) system controller executes a finite rotation of peristaltic pump (e.g., 1/3 revolution, 1 revolution, 5/3 revolution, etc.), (ii) pressure transducer measures the resulting decrease and increase in pressure at or near the catheter or peristaltic pump inlet, (iii) mathematical techniques such as Time-Domain Viscometry (see inventor's co-pending applications listed as references) are utilized to measure the viscosity of the aspirate within the catheter, (iv) aspirate samples which are viscometrically similar or identical to blood may be returned to the patient bloodstream (by reversing peristaltic pump through a finite rotation) thereby eliciting a measurement of aspirate viscosity with zero or near-zero blood loss. Viscometric sampling may also be utilized by embodiments and methods of the present invention to measure the distance between a catheter tip and a thrombus; this may be termed viscometric distance sampling and is presented in conjunction with FIG. 3A through FIG. 3C.

Continuously or intermittently measuring the aspirate viscosity during a thrombectomy procedure enables a control system (and/or clinician, technician, remote observer, etc.) to discriminate between effective and ineffective treatment regimens (e.g., thrombectomy operating modes, combination of system setpoints, etc.). Experimental data (e.g., aspirate viscosity, relative flow rate, % thrombus, blood pressure, etc.) may be concurrently, intermittently, continuously and/or post-procedurally correlated to each treatment regimen attempted or executed during the procedure. Within each procedure, each experimentally attempted treatment regimen (e.g., a combination of system setpoints, thrombectomy operating mode, manual manipulation, etc.) may be executed and immediately evaluated for thrombectomy efficacy and to infer subsequent treatment regimens. Post-procedurally, data from each procedure (e.g., procedure log file, etc.) may be contributed to a local or remote database for preservation and/or analysis; a compilation of post-procedural data may then be utilized to update system software or firmware with continuously-improving thrombectomy control algorithms or control strategies. Thrombectomy procedures and devices (e.g., catheter French size, length, etc.) vary widely in properties including size by clinical indication (e.g., ischemic stroke, DVT or Deep Vein Thrombosis, Pulmonary Embolism or PE, Chronic Total Occlusion or CTO, collagenaceous, calcified, etc.); data from similar procedures (e.g., PE, 8 French, 85 cm length, etc.) may be analyzed independently from other procedures (e.g., DVT, 5 French, 135 cm length). Each procedure that is performed may draw from real-time experimental, as well as historical, data to infer effective thrombectomy operating modes, and each procedure may contribute to a database such that ongoing improvement to control systems (e.g., thrombectomy mode control algorithm, thrombectomy control flowchart, etc.) may be developed and implemented.

Embodiments of the present invention may comprise the oscillation of a column of liquid within a catheter. Liquid Column Oscillator (LCO) embodiments may be subdivided into a lower-frequency range (approximately 0.1 Hz to 50 Hz) and a higher-frequency range (approximately 20 Hz to 19,000 Hz). The lower-frequency range of a representative LCO is termed an LF oscillator which imparts oscillatory, cyclic or reciprocating fluid flow within a catheter. The higher-frequency range of a representative LCO is termed Harmonic Oscillator (HO) which imparts fluid vibrations in the sonic frequency range to induce phenomena including resonance and/or standing and traveling waves within a catheter. Some embodiments of an LCO generate a spectrum of frequencies (denoted herein as f) spanning subsonic (f<20 Hz) to sonic (20 Hz<f<19,000 Hz) frequencies. LF oscillator embodiments typically impart finite displacement, velocity and acceleration of fluids within a catheter. Harmonic Oscillator (HO) embodiments typically impart standing or traveling sound waves to fluid within a catheter. LF oscillator embodiments are depicted herein as a reciprocating piston in a cylinder, and HO embodiments are depicted as a sonic transducer, hydrophone, submersible speaker, transceiver, transmitter and/or receiver wherein a vibrating diaphragm (or analogue) induces and/or detects oscillatory pressure fluctuations within a fluid in a catheter.

Embodiments of the present invention may comprise a LF oscillator to deliver oscillatory flow to the catheter tip, whereupon the oscillatory flow may directly impinge upon the thrombus, transferring oscillatory fluid momentum to the thrombus. The LF oscillator intermittently discharges and subsequently withdraws a finite volume of fluid to/from the proximal end of the catheter, generating a forced oscillation of fluid within the catheter. The oscillatory flow produces a sequence of forward/reverse (antegrade/retrograde) flow and subsequent forces upon an intravascular site generally distal to the catheter tip. This sequence of antegrade/retrograde flow generates (1) bi-directional erosive flow forces at the surface of the thrombus (surface forces), and (2) oscillatory forces tending to push and pull the thrombus. The frequency range of an LF oscillator may range from approximately 0.1 Hz to 50 Hz. The LF oscillator therefore is operable in the subsonic to low sonic frequency ranges; the frequency may be intermittently or continuously adjusted such that preferred frequencies (i.e., frequencies that effect thrombus disruption and aspiration) may be identified. Preferred embodiments of an LF oscillator comprise adjustments to the frequency and/or amplitude of the oscillatory fluid motion induced within catheter; frequencies and/or amplitudes may be incrementally or continuously adjusted during the course of a thrombectomy procedure. As efficacious or preferred frequencies are identified, these preferred frequencies may be explored and/or exploited to any limit of diminishing returns.

Embodiments of the present invention may independently and further comprise a Harmonic Oscillator (HO) which may comprise generally higher frequencies and/or utilization of resonance phenomena within a fluid filled catheter that may augment the (fluid mechanical/acoustic) power delivered to the catheter tip. The fluid filled catheter may be modelled as an organ pipe or guitar string; these models illustrate standing wave resonance just as a piano or a pipe organ fills a concert hall with sound. The frequency range of an HO may range from approximately 20 Hz to 19,000 Hz; the HO is therefore operable in the sonic or audible frequency range. Resonance and/or standing wave phenomena enable embodiments of the HO to act analogously to an organ pipe or piano string to contain, channel, transmit, emit or otherwise transfer hydrodynamic energy/power which may impinge upon tissue including thrombi and surrounding vasculature. Generation of standing wave phenomena (which augment the amplitude of oscillations) may generally occur at one more discrete harmonic (natural, first or fundamental) frequencies (and overtones or higher order harmonics). A sequential plurality of approximately such frequencies may be explored for thrombectomy efficacy. Frequencies other than harmonic/natural (e.g., out of phase, off-harmonic, etc.) may typically produce traveling wave phenomena and may provide efficacious outcomes as well. Preferred embodiments of an HO may comprise adjustments of process variables including harmonic oscillator input frequencies (HO frequencies) adjusted to a plurality of values including harmonic and/or off-harmonic setpoint values.

Embodiments of the present invention may independently and further comprise non-oscillatory, unidirectional infusion of fluid for improved thrombectomy efficacy. Infused fluids (e.g., saline, lubricant, thrombolytic or other therapeutic agent, etc.) may be discharged internally within a catheter (to a proximal, distal or any intermediate location with respect to the catheter) and may also be discharged externally, generally directed into the bloodstream. Infused fluid may be pressurized to a (pressure or flow) setpoint level such that appreciable fluid pressure and/or momentum of infused fluid may be available to do work in the form of thrombus maceration, fragmentation, degradation, ablation, attrition, dislodgement, disintegration, etc.; infused fluid may enable rapid clearing of thrombus within a catheter. Depending upon the thrombectomy system embodiment, clinical indication (e.g., ischemic stroke, DVT, PE, etc.), catheter selection, etc., infusion tubes may be fixed or moveable (with respect to the catheter). A moveable infusion tube may be extended distally (from the catheter tip) to form a hydrodynamic and/or mechanical lance to pierce, probe, penetrate, dislodge or otherwise effect thrombus aspiration. A moveable infusion tube, acting as a hydrodynamic and/or mechanical lance, may be deployed in a manner similar to an obturator or separator. Moveable infusion tube may be repositioned, translated, rotated or moved by automated, mechanized, or manual means.

Embodiments of the present invention may comprise adjustment (e.g., of levels, setpoints, settings, etc.) of process variables or factors (e.g., LF oscillator frequency and/or stroke, HO driven frequency, HO resonant frequency, aspiration rate, infusion, infusion pressure, catheter/infusion geometry, etc.) and subsequently implement feedback and/or feed-forward control to identify, explore and/or exploit any characteristic combination of process variables that evoke desired or favorable system responses (e.g., increased % thrombus in aspirate, aspirate viscosity<750 cP, etc.).

Embodiments of the present invention may comprise concurrent and independent setpoint control over a plurality of system process variables (thrombectomy operating modes) and concomitantly or subsequently analyze/assess combinations of setpoints which are effective in aspirating thrombus; thrombus aspiration efficacy (thrombectomy efficacy) may be measured viscometrically. Viscometric data may be correlated to thrombectomy operating modes (e.g., LF oscillator=10 Hz, H0=750 Hz, aspirate pump speed=10RPM, etc.) to discriminate between effective and ineffective thrombectomy operating modes during a thrombectomy procedure. At any time (e.g., during or after a procedure) a data log of thrombectomy efficacy vs. thrombectomy operating mode may be available to any or all or the following: (i) the thrombectomy mode control flowchart or algorithm (feedback control), (ii) the clinician (feedback and input), (iii) a remote observer, (iv) an historical database which may be compiled and analyzed in order to enable revision or updates to software or firmware including the thrombectomy mode control flowchart or algorithm. Embodiments of the present invention enable thrombectomy systems to (a) measure procedure efficiency (thrombectomy efficacy) in real-time, (b) test and evaluate a plurality of thrombectomy operating modes for efficacy, (c) generate and retain correlation data for utilization including intra-procedural, inter-procedural, post-procedural software/firmware updates, etc.

Embodiments of the present invention include LF oscillator and HO featuring adjustable system process parameters or variables; similarly, variable infusion and catheter/infusion geometry configurations are disclosed which provide additional system process variables which may be controlled under setpoint, level, manual or other means. In general, increasing the number of system process variables that may be controlled or adjusted may be advantageous in a thrombectomy system because specific combinations of those process variables may produce procedural results exceeding those attainable by any individual contributor acting independently.

Some embodiments of the invention utilize viscometric distance sampling to detect and/or measure the distance between a catheter tip and a target thrombus. Furthermore, embodiments of the present invention subsequently limit the magnitude of aspiration vacuum/suction to levels corresponding to the measured distance. This provides at least two distinct advantages over prior art: (1) blood loss is minimized and (2) likelihood of clogging or corking a catheter is minimized. Blood loss is minimized by limiting the aspiration vacuum/suction levels in configurations wherein the catheter tip to thrombus distance is “too large” (range of approximately 3 to 10 times the catheter diameter); in such configurations, blood would be predominantly aspirated without successful harvesting of thrombus. The likelihood of clogging or corking a catheter is minimized by limiting the aspiration vacuum/suction levels corresponding to the “thrombus load” or % thrombus within the catheter; the catheter may be repositioned to keep the thrombus load to a level sufficiently low for aspiration by differential pressure means (i.e., vacuum/suction).

Some embodiments of the invention utilize a methodology wherein each thrombectomy procedure is an exercise in thrombus removal; the desired outcome of the exercise is to transport a maximum amount of thrombus through a catheter in a minimum amount of time. Each thrombectomy procedure (or exercise) may be comprised of a number of discrete steps including, as examples:
1. deploying and advancing a catheter to a thrombotic location,
2. determining and achieving a proper proximity between the catheter and a target thrombus,
3. executing a plurality of thrombectomy operating modes and/or techniques (conducting experiments or deterministic events),
4. assessing the efficacy of each thrombectomy operating mode and/or technique executed (measuring the outcomes of the experiments or deterministic events), and
5. repeating or altering thrombectomy operating modes until a procedure endpoint is determined or established.

Some embodiments of the present invention comprise instrumentation (e.g., pressure measurement, viscosity measurement, etc.) to facilitate steps 2 through 5 of the above list. Steps 3 and 4 (of the above list) may be executed concomitantly such that as a thrombectomy operating mode is executed it is quantitatively assessed for measured physical properties of the aspirate. Thus, a thrombectomy procedure may be decomposed to comprise a sequence of discrete experiments (i.e., thrombectomy operating modes) wherein a definitive, quantitative experimental result (e.g., 20% increase in viscosity to 25 cP, 10% reduction in relative flow rate to 80%, etc.) may be correlated to each thrombectomy operating mode executed or experiment conducted. The examples given are representative of efficacious thrombectomy operating modes because the amount of thrombus (% thrombus, thrombus load) within the catheter is measured to have increased as a result of a thrombectomy operating mode that predicated the outcome of the measurement.

Embodiments of the present invention may also comprise a systematic approach to the selection of thrombectomy operating modes to be executed (i.e., experiments to be conducted) and also a determination of the order that experiments are conducted. The standard techniques of DOE (Design of Experiments) may be employed or adapted including: full factorial, fractional factorial, Taguchi, etc.; experimental measurement data are rapidly available for feedback such that any effective thrombectomy treatment regimen (e.g., thrombectomy operating mode, combination of system setpoints, device manipulation, etc.) may be repeated, altered or otherwise exploited for efficacious thrombus aspiration. The standard techniques of SPC (Statistical Process Control) may be used to determine the significance of any measurement data (e.g., thrombectomy efficacy, viscosity, relative flow rate, 5% thrombus, etc.) collected over a plurality, array or sequence of experiments (thrombectomy operating modes).

Any or all events resulting from a (particular) thrombectomy operating mode (e.g., combination of factor/level, setpoints, adjustment, manual manipulation, etc.) may be analyzed for thrombectomy efficacy using instrumentation including pressure transducers, viscometers, flowmeters, etc. From the standard techniques of DOE, the adjustable system variables are termed factors and the plurality of setpoints available for each factor is termed levels. An example of a one factor, two level (thrombectomy operating mode) experiment design is vacuum/suction on or off; two experimental states are available. A one factor, two level experiment is the limit of some prior art thrombectomy systems commercially available, such as an evacuated reservoir and a two-position (on/off) valve.

An example four factor (e.g., LF oscillator frequency, LF oscillator stroke, HO frequency, infusion rate, etc.), six level (e.g., 5 Hz to 20 Hz, 1 mm to 5 mm, f≈375nHz, 1 to 3cc/s, etc.) thrombectomy operating mode matrix or array may present approximately 1,300 experimental states available for analysis (in a full-factorial experiment design). Experiments with fewer experimental states may be constructed by standard means including fractional factorial or Taguchi experiment design. Experiments with fewer experimental states may be constructed by selecting efficacious factors, levels, initial values and ranges (of levels) by means including post-procedural analysis of thrombectomy procedures (historical data). Post-procedural analysis may manifest as updated software or firmware or as clinician-selected factors, levels, initial values, ranges, etc. based upon expertise, experience, training, published data, etc.

An example of post-procedural analysis is given as follows: Consider a three factor (system X, system Y and system Z), ten level experiment (setpoints range from 1 to 10). For example, system X setpoint may be LCO frequency, system Y setpoint may be HO frequency and system Z setpoint may be aspirate pump speed. A shorthand notation for a sequence of setpoints is: (1,1,1), (1,1,2), (1,1,3), . . . (1,2,1), (1,2,2), . . . (2,1,1), (2,1,2), . . . (10,10,10). Each of the 1000 setpoints are experimentally executed in a first procedure, with only certain experiments being positively correlated to thrombus in the catheter. The correlation data of the first procedure revealed that all of the setpoints above 6 were not positively correlated, and the combination (3,3,3) positively correlated at the highest significance level. In an example second procedure, each setpoint X, Y and Z is changed to range from 1 to 6; the number of experiments is reduced from 1,000 to 216 (=6³). In the second procedure, the initial values are assigned the value of (3,3,3). In the second procedure, the experiment sequence is changed to: (3,3,3), (3,3,4), (2,4,3), (4,3,3), . . . and eventually to (1,1,1) or (6,6,6), termination, or a variation thereof. By changing the range of setpoints and the initial values of the apparatus, the efficiency of the second procedure is improved over the first procedure. In some embodiments, post-procedural analyses such as this may be conducted manually (observation of data trends and changing values such as range, increment, initial values, etc.); in some embodiments the averages of many procedures may comprise the dataset. In some embodiments, changes made to parameters such as range, increment, initial values, arbitrary constants, threshold values, etc. are enacted by manual or automated means such as software. In software embodiments, data may be sorted based upon thrombectomy efficacy; this identifies a subset of the data (from previous procedures) and identifies efficacious systems and setpoints (factors and levels). Some embodiments employ statistical analysis; statistical parameters such as mean and standard deviation may be used to determine initial values and ranges for setpoints. Some embodiments utilize thrombectomy control algorithms that identify efficacious experiments, store the setpoint values in memory, and repeat efficacious experiments. Some thrombectomy control algorithms identify efficacious experiments, and invoke a fixed or variable dwell period (of operation) such that efficacious experiments are being conducted for longer periods of time over the course of the procedure.

Current generation, commercially-available thrombectomy systems generally do not feature such an experimental approach because these thrombectomy systems generally do not feature: (1) viscometric sampling, a quantitative measurement of viscosity/flow rate/thrombus concentration (thrombectomy efficacy) within the catheter, (2) a significant plurality of thrombectomy operating modes, and (3) a control strategy (e.g., thrombectomy control flowchart, algorithm, etc.) wherein a significant number thrombectomy operating modes are executed, with the thrombectomy operating modes being quantitatively assessed for thrombectomy efficacy. Some embodiments of thrombectomy systems of the present invention feature an experimental approach to the cause—effect relationship between discrete thrombectomy operating modes and thrombectomy efficacy. Cause—effect analysis may generally only be developed in conjunction with instrumentation such as viscometric sampling of aspirate to determine thrombus load. Prior art thrombectomy techniques or experiments (conducted in the absence of relevant quantitative measurement data) may allow only “observations” which may be “monitored” for change. Some example prior art thrombectomy systems generate tones, sounds or colored lights based upon pressure measurements which are audible and/or visible to the clinician. As the measured pressure “changes,” the sound volume and/or pitch, and/or light color, is also “changed.” It is left to the clinician to interpret any changes in the volume, pitch or light color. Prior art may also include a visual display which changes images or colors in response to changes in measured pressure.

The present disclosure supplements the inventor's co-pending applications (listed as references) wherein any thrombectomy system comprising aspiration and adjustable system process variables (e.g., pump speed, vacuum level, manual catheter or device manipulation, infusion rate, rotating cutter speed, thrombolytic agent infusion, etc.) may be improved by viscometric measurement and/or physical property measurement/analysis of the aspirate as a diagnostic aid and/or feedback mechanism. Real-time measurement data (e.g., viscosity measurement, relative flow rate measurement, % thrombus, etc.) enable the construction of a thrombectomy control flowchart or algorithm which: (1) provides clinician feedback to detect the presence of and/or proximity to a thrombus (at a current site), (2) adjusts at least one system process variable, (3) quantitatively (e.g., viscometrically, volumetrically, etc.) assesses the effect of the adjustment(s), and (4) arrives at a data-driven decision {e.g., conditional statement IF (μᵢ₊₁>μᵢ, where μᵢ is the viscosity of the i^{th} iteration) THEN (adjust system process variable j), etc.}, where the aspirate viscosity after the adjustment(s) may again be compared to the viscosity before the adjustment(s). Commercially available thrombectomy systems may depict rudimentary examples of such a flowchart, however the shortcomings include: (1) the flow rate of aspirate through of the catheter is assessed in attribute data terms (e.g., full flow, clogged, free-flow, etc.) rather than in variable data terms (e.g., 87% relative flow rate, 2.6cc/second, etc.) and (2) the number of adjustable system process variables may be generally rudimentary or even binary (e.g., on-off, open-closed, etc.). The present disclosure includes embodiments simultaneously controlling (an example number of) seven independent system process variables (factors) in an example nested-loop flowchart; other embodiments may include other control schemes including: Design of Experiments (DOE), fuzzy-logic, Proportional Integral Derivative (PID), etc. Statistical Process Control (SPC) may be integrated in order to assess the statistical significance of any measured change in thrombectomy efficacy. Control system embodiments of the present invention may be implemented requiring a minimum of hardware, computational resources and programming skill.

The geometry of a typical catheter is suitable for the implementation of either (or both) an LF oscillator and/or an HO; in both cases because catheters exhibit a large ratio of Length/Diameter (L/D, or Aspect Ratio). Any fluid filled conduit exhibiting an AR greater than approximately 50 is a candidate for an LF oscillator or HO. As an LF oscillator, typical catheter geometries enable a generally small mass (range of approximately 0.001 g to 50 g) of fluid to be reciprocally accelerated and decelerated to sufficient velocity such that the magnitudes of fluid momentum and kinetic energy (of the oscillating fluid) developed may be sufficient to fluid-mechanically disrupt thrombus near the distal end of the catheter. Within the conduit, the oscillating mass of fluid intermittently exchanges kinetic energy and potential energy; this potential energy may comprise pressure energy and system compliance including elasticity and deformation of components. Intermittently, a mass of fluid is discharged from a catheter into a remote fluid reservoir (e.g., vein, artery, bladder, tank, etc.) at sufficient velocity such that momentum and kinetic energy are available to do work. Momentum is a vector, and the direction of discharge is generally in the direction of the catheter axis at the tip; however, embodiments of the present invention include a non-axial discharge, thereby producing lateral forces on the catheter. The geometry of a catheter is well-suited to channel, constrain and direct high-velocity, high (or low) pressure fluid flow to any fluid or surface (e.g., blood, thrombus or vasculature, etc.) in close proximity to and generally axially aligned with the catheter tip. From a resonance-phenomena perspective, a catheter may be analogously compared to an organ pipe, wherein the sound emitted may be directional and may occur near the pipe opening, or a piano string wherein the pegs experience oscillatory translational forces superposed over the static force of string tension.

Surface forces resultant from oscillatory flow generally past a thrombus may erode, dislodge and entrain material from the surface of the thrombus, creating a blood/thrombus slurry which may be aspirated by suction or vacuum. Oscillatory flow may exist superposed over non-oscillatory flow (as may be generated by aspiration vacuum or suction) such that there is a net flow of aspirate into a collection vessel of the thrombectomy system. With both a LF oscillator and an aspiration system active, any blood or thrombus contained within the catheter may experience oscillatory flow with a net flow out of the patient and into the collection vessel. Vacuum may be provided to the catheter by means including an evacuated reservoir or a liquid pump such as a peristaltic pump. A peristaltic pump may be advantageous (over an evacuated reservoir) because the vacuum level may be controlled by changing the speed of the pump shaft. The vacuum level may be adjusted to a desired value by feedback from a pressure transducer in fluid communication with the peristaltic pump inlet; the desired vacuum level may be attained by a system controller or manual manipulation of a speed control knob. In some embodiments, the aspirate pump speed may be continuously or intermittently adjusted in response to measurements including the viscosity of the aspirate contained within the catheter.

An example LF oscillator embodiment may comprise an oscillating or reciprocating piston in a cylinder that is fluidically coupled to a catheter lumen, thus creating oscillatory or reciprocating fluid flow within the catheter lumen. The piston may be generally described as surface (or a moving surface) which is moving in oscillatory or reciprocating fashion. The piston oscillation or reciprocation may be imparted by means including a crankshaft and connecting rod, as in a piston pump; a piston pump with a single, valve-less intake/discharge port represents an embodiment of a prime mover of fluid in representative LF oscillator embodiments. In alternative embodiments to a crankshaft and connecting rod, the moving surface oscillation may be enacted by means including a linear actuator, rotating cam mechanism, solenoid, Scotch yoke, etc. An LF oscillator may be comprised of an oscillating diaphragm or other physical barrier to fluid flow. An LF oscillator may be comprised of a section of tubing which is intermittently compressed and released by surface forces (e.g., squeezing the tubing between moving surfaces or hydrostatic compressions, etc.). The oscillation may be that of a simple harmonic oscillator (a sinusoidal wave), a square wave, a sawtooth wave or other waveform; the frequency of oscillation may be biased for a longer duration of either the intake or exhaust stroke of the piston or diaphragm.

In some embodiments, the frequency and the stroke of the LF oscillator may be adjusted or selected to effect a desired velocity, pressure and acceleration of the fluid within the catheter. On each stroke of an LF oscillator, the pressure or vacuum that is developed is not only a function of the frequency and stroke, but also parameters including (1) the dimensions of the catheter and (2) the physical properties of the fluid (viscosity, density, etc.). A more dense or viscous fluid may develop a greater pressure because momentum and viscous forces impede flow into or out of the catheter. On the intake stroke of an LF oscillator, the pressure (negative pressure/vacuum) that is developed may be limited to the vapor pressure of the fluid. If the pressure in the LF oscillator or catheter decreases below the vapor pressure of the fluid, cavitation and/or boiling (vaporization of liquids) may occur.

For an LF oscillator comprising a reciprocating piston in a cylinder, the theoretical maxima of amplitude, velocity and acceleration of a massless, inviscid fluid oscillating within the catheter are given by Eq. 1, Eq. 2 and Eq. 3.

$$Maximum Theoretical Catheter Flow Amplitude = \frac{{( LCO Bore )}^{2}}{{( Catheter ID )}^{2}} \times LCO Stroke \qquad Eq . 1$$

$$Maximum Theoretical Catheter Flow Velocity = π f \frac{{( LCO Bore )}^{2}}{{( Catheter ID )}^{2}} \times LCO Stroke \qquad Eq . 2$$

$$Maximum Theoretical Catheter Flow Acceleration = 2 {π}^{2} {f}^{2} \frac{{( LCO Bore )}^{2}}{{( Catheter ID )}^{2}} \times LCO Stroke \qquad Eq . 3$$

Where f is the frequency and may be expressed in units including Hz or cycles per second. In reducing the invention to practice, the maximum values of Eq. 1, Eq. 2 and Eq. 3 are generally not attained (at operational frequencies) due to physical phenomena including fluid mass, compressibility, elasticity, vapor pressure of solid and/or fluid components of the system.

As an example, a representative 9F catheter has approximate dimensions of 3 mm OD and 2.5 mm ID and 100 cm length, the cross-sectional area of the catheter bore is approximately 5×10⁻⁶ m². A representative example LF oscillator embodiment has a 1 cm bore and 1 cm stroke and is operated at 1 Hz (60 RPM); the volumetric displacement is approximately 0.8 ml/revolution. Substituting these values into Eq. 1, Eq. 2 and Eq. 3 yields Eq. 4, Eq. 5 and Eq. 6.

$$Maximum Amplitude = \frac{10 {mm}^{2}}{{( 2.5 mm )}^{2}} \times 10 mm = \frac{1000}{6.25} mm \approx 160 mm \approx 16 cm \approx 6.3 inch \qquad Eq . 4$$

$$Maximum Velocity = π \times 1 Hz \times \frac{10 {mm}^{2}}{{( 2.5 mm )}^{2}} \times 10 mm \approx 500 mm / s \approx 20 in / s \qquad Eq . 5$$

$$Maximum Acceleration = 2 {π}^{2} \times {( 1 Hz )}^{2} \times \frac{10 {mm}^{2}}{{( 2.5 mm )}^{2}} \times 10 mm \approx 3.1 m / {s}^{2} \approx 10 ft / {s}^{2} \qquad Eq . 6$$

The volume of fluid contained within the example catheter is approximately 5,000 mm³ or 5 ml; this analysis neglects the mass of fluid in the LF oscillator cylinder. The example 160 mm amplitude within a 2.5 mm ID catheter yields a theoretical volume transfer of approximately 0.8 ml/stroke (0.16 m×5×10⁻⁶ m²×10⁻⁶ cm³/m³), which has mass of approximately 0.8g. Representative fluids including blood, saline and thrombus have a density of approximately 1 g/ml; the mass of fluid in the example catheter is approximately 5 grams (range of approximately 1 gram to 50 grams). The example LF oscillator/catheter system therefore oscillates a 5-gram column of liquid through a distance of 16 centimeters two times per second, once in the distal direction and once in the proximal direction. In the example catheter/LF oscillator combination, the theoretical fluid within the catheter intermittently stops (to zero velocity) and accelerates (to approximately 50 cm/s velocity) two times during each cycle. Approximately 0.8g of fluid exits and 0.8g of fluid enters the distal tip of the catheter with each idealized LF oscillator cycle of one second duration.

The theoretical maximum pressure required to effect this example acceleration may be calculated by Newton's Second Law (Eq. 7) and the definition of pressure (Eq. 8).

Maximum Cyclic Force: F=ma=(0.005 kg)×3.1 m/s²=±0.016N Eq.7

Maximum Cyclic Pressure: P=F/area=±0.016N/5×10⁻⁶ m²≈±3,200 Pa≈±0.032bar Eq. 8

This theoretical pressure amplitude calculated in Eq. 8 is the cyclic pressure extrema corresponding to accelerating (oscillating) a frictionless (inviscid) fluid column. The ±symbol indicates that during the discharge stroke the pressure is positive (above ambient, atmospheric or intravascular pressure, etc.) and during the intake stroke the pressure is negative (below ambient, atmospheric or intravascular pressure, etc.). The distal end of the catheter may be exposed to a pressurized environment (e.g., atmospheric pressure or intravascular pressure when the device is deployed in the patient bloodstream, etc.). It is assumed that fluids (viscous or inviscid) will experience a pressure fluctuation around an intermediate pressure value. Herein it is assumed that atmospheric pressure is approximately 760 mmHg (1 bar, 100 kPa) and intravascular pressure is approximately 800 mmHg (1.05 bar, 105 kPa) absolute. The calculated pressure range for the example LF oscillator/catheter combination is 96.8 kPa to 103.2 kPa for a catheter containing and immersed in an inviscid fluid at approximately standard atmospheric pressure (760 mmHg, 100 kPa).

Viscous friction and PV work have been neglected herein, as was the mass of liquid in the LF oscillator cylinder; experimental pressures may therefore be different. As the negative cyclic pressure amplitude approaches full vacuum (≈−100 kPa gauge), the phenomenon of cavitation or boiling may occur wherein the cyclic pressure is calculated to be below the vapor pressure of the fluid contained within the LF oscillator/catheter combination. Setting Eq. 8 to 100 kPa and solving for frequency in Eq. 6, we find that a frequency of approximately 6 Hz is calculated to be great enough to theoretically induce cavitation or boiling in water, saline or blood; cavitation or boiling may occur at frequencies less than 6 Hz because of viscous losses and PV work. The example representative catheter and LF oscillator combination produces oscillatory fluid displacement and velocity values that may not be practical for an LCO thrombectomy system comprising an LF oscillator. Other values for the LF oscillator stroke and frequency may be calculated from Eq. 1 and Eq. 2 or experimentally determined.

Cavitation or boiling may occur on the inlet stroke of an LF oscillator/catheter combination, depending upon system process variables including frequency. Cavitation or boiling has three important consequences: (1) the actual (negative portion of) intra-catheter fluid amplitude will be diminished to some fraction of theoretical, (2) the minimum attainable pressure (the vapor pressure of the aspirate) is intermittently applied to a proximal portion of the catheter, and (3) as the vapor bubble collapses to an incompressible fluid, an axially-directed shock wave may be transmitted through the catheter, such that intravascular tissues (including thrombus) are subjected to intermittent shock effects which may be emitted, radiated or projected outward from the distal end of the catheter. Item (1), above, ensures that the maximum attainable amplitude is achieved for any cavitating (boiling) system. Item (2) ensures that the minimum cyclic pressure is achieved with each cycle. Item (3) provides a distally-directed shock wave to disrupt, dislodge, erode, disintegrate or otherwise disturb intravascular thrombotic material distal to the catheter tip. The resulting shock may also be effective in clearing a clogged or corked catheter. Cavitation therefore provides (1) maximum attainable liquid column amplitude, (2) maximum attainable vacuum to dislodge and aspirate thrombus, and (3) a liquid-column shock wave (similar to water hammer) directed to impinge upon the surface of thrombus in the distal proximity of the catheter tip. This shock wave may impart impulse forces, surface (erosive) forces and oscillatory (momentum-inducing/rocking) forces upon any thrombus.

Embodiments of a generalized LCO thrombectomy system featuring an LF oscillator may be modeled as a non-isovolumetric system which is closed at the proximal end (e.g., piston and cylinder) and open at the distal end (e.g., the catheter tip); fluid transfer into and out of the distal end of the catheter generally results from the differential pressure between opposite ends of the catheter. A thrombectomy system which features a catheter that is “long,” “of small diameter,” and filled with a “viscous” and/or “dense” fluid will generally exhibit a lower flow rate (absolute and relative) than one featuring a catheter which is “short,” “of large diameter,” and filled with an “inviscid” and/or “low-specific gravity” fluid. System parameters including catheter length and diameter, fluid viscosity and density, etc. therefore also influence the onset of cavitation. Cavitation may occur under conditions wherein the time rate of change in system volume exceeds the flow rate afforded by parameters including: catheter length and diameter, fluid viscosity and density.

The foregoing provides examples of oscillatory or reciprocating flow which may be utilized to disrupt and aspirate a thrombus at frequencies which may range from approximately 0.1 Hz to 50 Hz; at the higher end of this frequency range, smaller displacement volumes (smaller bore and/or smaller stroke) may be necessary to avoid unwarranted cavitation/boiling and/or system overpressure. A generalized thrombus, adhered or otherwise affixed to a vessel wall may exhibit one or more natural frequencies of vibration; exciting any of these natural frequencies may elicit a desired result of thrombus dislodgement, fragmentation, disintegration, ablation, etc. Some commercially available thrombectomy systems employ pulsatile flow at ultrasonic frequencies ranging from approximately 20 MHz to 30 MHz with the intent of finding a natural vibrational frequency of a thrombus; the approximate wavelength for ultrasonic pulsations or flow is approximately 75 μm to 50 μm. Equations governing the example LF oscillator predicted cavitation or boiling at approximately 6 Hz, corresponding to a wavelength of approximately 250m.

Embodiments of the present invention comprise utilization of resonance phenomena in at least three distinct ways: (1) resonance of a thrombus, (2) resonance within a catheter and/or (3) resonance of the vasculature. Large-scale resonance of a thrombus may be initiated by low-frequency/large amplitude fluid oscillations which may be imparted by an LCO thrombectomy system featuring an LF oscillator; the LF oscillator frequency may be discretely or continuously adjusted throughout a range (e.g., approximately 0.1 Hz to 50 Hz). Natural resonant frequencies of thrombi are generally unknown a-priori and may be experimentally determined by means including viscometry or flow measurement that provide feedback as the system approaches resonance or other efficacious state. Resonance within a catheter may generally comprise standing or traveling wave phenomena which are well-understood and provide mathematical models to approximately predict specific frequencies at which maximum amplitude and power conditions are likely to exist. Resonance of the vasculature may elicit cyclic distortions of vessel walls.

Resonance of a thrombus: axial oscillations of the column of fluid within the catheter may occur regularly such that oscillatory motion of the liquid may be used as a model. An equation for forced, damped harmonic motion is given in Eq. 9.

m\ddot{x}+μ\dot{x}+kx=A cos(ωt) Eq. 9

Where m represents the oscillating mass, μ represents linear frictional losses such as fluid viscosity and k represents a spring constant. \ddot{x}, \dot{x} and x are the acceleration, velocity and position of the mass (e.g., thrombus, blood, saline, etc.). The right-hand-side term, A cos(ωt), is the forcing function, which may be provided by an LF oscillator or an HO such as an oscillating piston in a cylinder or an oscillating or vibrating diaphragm, etc. The general solution to Eq. 9 is given in Eq. 10.

x(t)=B cos(ω₀t+ϕ)+Ce^{-γt/2} cos(ω′t+δ) Eq. 10

Where B and C are amplitudes of the oscillating mass and ϕ and δ are phase angles. Eq. 11 relates the physical system parameters (u., k and m) to relevant terms of Eq. 10, which is the general solution to Eq. 9;

$${ω}_{0} = \sqrt{\frac{k}{m}} ; γ = \frac{µ}{2 m} \qquad Eq . 11$$

where ω₀ is a natural frequency, γ is a damping coefficient, k is a spring constant, m is the mass or density of an oscillating mass and μ is damping which may arise from fluid viscosity or other frictional phenomena. The amplitudes (B and C), phase angles (ϕ and δ may) and frequency ω′ may be obtained by standard methods, if desired.

Eq. 11 provides insight to implementation of the present invention because the mass of any representative thrombus, while unquantified by the system controller and/or clinician, may typically lie within a range of approximately 0.05 g to 50 g. Eq. 11 also provides that larger masses oscillate at natural frequencies that are lower (i.e., smaller) than those of smaller masses. Therefore, a system controller and/or clinician detecting a large thrombotic mass may elect to explore the lower ranges of frequencies. The spring constant, k, also provides insight to the nature of the thrombus structure and its attachment to a vessel wall. A more elastic attachment, characteristic of fresh thrombus, is characterized by a smaller value of k; in contrast, a more rigid/brittle attachment, characteristic of chronic thrombus, is characterized by a larger value of k. Therefore, a system controller or clinician detecting a fresh, elastic thrombus (k is smaller) may elect to explore the lower frequency ranges for improved thrombectomy efficacy. A system controller and/or clinician detecting a chronic thrombus (k is larger) may elect to explore the higher (i.e., larger) frequency ranges for improved thrombectomy efficacy. Eq. 11 further provides insight that one or more natural frequencies of internal vibrational modes may be at a higher frequency than the resonant frequencies of the total mass of thrombus. This is because a 1g mass of thrombus may be modeled as an arbitrary quantity of lesser masses interconnected by elastic attachments (e.g., quantity 1000 individual masses of 1 mg each, etc.). Each of these example 1000 masses is in close proximity to near neighbors so k may be inferred to be large. Natural frequencies of internal vibrational modes may be inferred to exist at higher frequencies than a single oscillating mass. Some embodiments of the present invention span sufficient frequency ranges to potentially excite multiple vibrational modes of a thrombus for improved therapeutic efficiency and efficacy.

Resonance and standing wave phenomena within a catheter, the Harmonic Oscillator (HO): a catheter may be modeled as an organ pipe during instances where the distal end of a fluid-filled catheter is unoccluded and in fluid communication with the surrounding tissues, e.g., the patient bloodstream. Alternatively, a catheter may be modeled as a piano string (or an acoustic cavity closed at both ends) during instances where the distal end (or any portion of the catheter) of the catheter is occluded or clogged. The oscillating medium may comprise components including saline, blood, thrombus, etc. or any mixture thereof; the density of the oscillating medium is taken to be approximately 1g/cc (1,000 kg/m 3) and the viscosity is generally in the lower end of the range between 1 cP to 1,000 cP (1mPa·s to 1,000mPa·s). The speed of sound of saline or blood is taken herein to be approximately 1,500 m/s. An objective of a thrombectomy procedure is to aspirate thrombus, some portions of which may be approximately the size (diameter) of the catheter; this may lead to clogging or “corking” of the catheter because of low pressure applied at the proximal end of the catheter. When open or unoccluded, the catheter may be modeled as a (1) closed-open acoustic chamber (organ pipe, trumpet, etc.); when clogged or occluded, the catheter may be modeled as (2) a closed-closed acoustic chamber (e.g., piano string, guitar string, etc.). Standing waves may occur in a catheter whether or not the distal end is occluded or clogged; however, some frequencies of the standing waves may be different in the two cases.

Herein, a generally sinusoidal, oscillatory motion is assumed to be imparted to a column of fluid contained within a catheter. Such motion may be modeled as a forced, damped harmonic oscillator with a moving boundary condition (piston, diaphragm, speaker cone, transducer, etc.). The equation for the fundamental and overtone/harmonic frequencies for a pipe closed at one end is given in Eq. 12; however, only odd harmonics are allowed for such a system. The first few harmonics (including “overtones”) are calculated for the example catheter which is not clogged.

$${fo}_{n} = \frac{nv}{4 L} ; {fo}_{1} = \frac{( 1 ) ( 1 , 500 )}{4 ( 1. )} \approx 375 Hz ; {fo}_{3} \approx 1 , 125 Hz , \qquad Eq . 12$$

$${fo}_{5} \approx 1 , 875 Hz , {fo}_{7} \approx 2 , 625 Hz , …$$

Where fo is a resonant frequency of a catheter open at the distal end, n is the n^{th} harmonic, v is the speed of sound in water (e.g., 1,500 m/s) and L is the catheter length (e.g., 1.0m). 375 Hz is approximately F^{#}above middle C (F^{#}4) and 2,625 Hz is approximately E7 or E three octaves above middle C; this frequency range is in the audible (sonic) frequency range. Exciting a 1m column of liquid water (with one end closed) at a suitable frequency may cause standing waves of approximately 375 Hz, 1,125 Hz, 1,875 Hz, 2,625 Hz, 3,375 Hz, etc. to form within the liquid column. If the liquid column were longer, the frequencies would be shifted downward (to lower values); if the liquid column were shorter, the frequencies would be shifted upward (to higher values). Embodiments of the present invention comprise a catheter of variable length in order that additional or alternate resonant frequencies may become existent by changing the catheter length.

The LF oscillator and HO embodiments disclosed herein comprise oscillatory motion and mechanical or pressure waves of the contents of a catheter as well as that of tissue proximate to the catheter tip; the frequency range is approximately 0.1 Hz to 19,000 Hz. At the lower end of this frequency range (e.g., LF oscillator subsonic and low sonic frequencies of approximately 0.1 Hz to 50 Hz) a finite fluid displacement may be calculated, observed, measured or quantified as presented in Eq. 1 through Eq. 8; an operational range for oscillatory fluid translation may be approximately 0.5 mm to in excess of 500 mm. At the higher end of the frequency range (e.g., HO, sonic frequencies exceeding approximately 20 Hz) any fluid displacements (x) may be characterized as infinitesimal (e.g., approximately 0<x<0.5 mm); the fluid field may be characterized as interspersed regions of compression and expansion (nodes and anti-nodes). The nodes and anti-nodes thereof comprising approximately local pressure extrema (e.g., maxima and minima). While fundamentally similar, the LF oscillator may be considered to transmit momentum waves through a catheter whereas the HO may be considered to transmit pressure or mechanical waves through a (generally stationary) medium in a resonating cavity. In both cases (LF oscillator and HO), the fluid medium (saline, blood, thrombus, clot, etc.) undergoes finite or infinitesimal translation (e.g., moves, undergoes motion, etc.). A fluid column acted upon by an (1) an aspirate pump, (2) an LF oscillator (operating at larger amplitudes) and (3) an HO (operating at higher frequencies) may exhibit the additive effects of (1) net flow, (2) oscillatory flow and (3) sonic pressure waves.

When a catheter becomes clogged (e.g., during the course of a thrombectomy procedure, testing, calibration, etc.) the organ pipe model may no longer apply and a piano string model may be applied. The equation for the fundamental and overtone/harmonic frequencies for a piano string or a pipe closed at both ends is given in Eq. 13; both even and odd harmonics are allowed for this system. The first few harmonics (including “overtones”) are calculated for the example catheter which is clogged at the distal end.

$${fc}_{n} = \frac{nv}{4 L} ; {fc}_{1} = \frac{( 1 ) ( 1 , 500 )}{2 ( 1. )} \approx 750 Hz ; {fc}_{2} \approx 1 , 500 Hz , \qquad Eq . 13$$

$${fc}_{3} \approx 2 , 250 Hz , {fc}_{4} \approx 3 , 000 Hz , …$$

Where fc is a resonant frequency of a clogged catheter, n is the n^{th} harmonic, v is the speed of sound in water (1,500 m/s) and L is the catheter length (1.0m). 750 Hz is approximately F^{#}one octave above middle C (F^{#}5) and 3,000 Hz is approximately F^{#}7 or F^{#}three octaves above middle C; also in the audible (sonic) frequency range. Exciting a 1m column of liquid water (with both ends closed) at suitable frequencies produces standing waves estimated to be approximately 750 Hz, 1,500 Hz, 2,250 Hz, 3,000 Hz, etc. to form within the liquid column. If the liquid column were longer, the frequencies would be shifted downward (to lower values); if the liquid column were shorter, the frequencies would be shifted upward (to higher values). A catheter may become clogged at a location more proximal than at the tip (e.g., 20 cm, 40 cm, 60 cm proximal to the tip, etc.); the length dimension of the resonance cavity is correspondingly decreased. Resonant frequencies for this clogged (at some intermediate location between the proximal and distal ends) catheter may be higher (i.e., shorter wavelength). In cases where a clogged catheter is detected, intra-procedural experimentation at higher than calculated frequencies may be conducted in an effort to clear a clogged catheter without the need for manual clinician intervention (e.g., guidewire, obturator, remove catheter, etc.). The input power to an HO may be measured at any time; as oscillations within a resonant cavity approach a natural frequency, an increase in input power may be measured. Embodiments of the present invention measure the input power to an HO and infer standing wave resonance at characteristic frequencies. Continued experimentation at these characteristic frequencies (at which input power to an HO is measured to be increased) may be utilized to disrupt, dislodge or otherwise cause a clogging thrombus to be successfully aspirated. Embodiments of the present invention may enable clearing of clogs within a catheter without time-consuming clinician intervention.

The first or fundamental frequencies for both an open and clogged representative catheter (approximately 1m in length, filled with water, saline, blood and/or thrombus) may be calculated to be not less than approximately 375 Hz; standing waves are not predicted to form at frequencies below 375 Hz. At fundamental and overtone harmonic frequencies standing wave phenomena may result in a reflection of waves at the aperture or the end of the resonant cavity (e.g., catheter tip), effectively containing the resonance within the catheter. This may cause the catheter to vibrate or oscillate, including in transverse directions; this phenomena may or may not be effective in transmitting wave (pressure) energy to a thrombus. At frequencies (less than or) intermediate between the resonance frequencies (e.g., as may be calculated by Eq. 12 and Eq. 13), standing wave phenomena may be insignificant and traveling wave phenomena may dominate. Off-resonance frequencies may radiate (i.e., emit) wave power effectively toward a thrombus for effective treatment. Identification of approximate resonance frequencies (e.g., as may be calculated by Eq. 12 and Eq. 13) provides guidelines for experimental exploration of frequencies which are calculated or observed to be resonant or off-resonant. Off-resonance frequencies which are approximately at the mid-point of calculated or approximated resonance frequencies may generate effective thrombectomy results because traveling waves may be radiated from, rather that reflected by the catheter tip. Multiple combinations of resonant and off-resonant frequencies may be experimentally conducted (and analyzed for thrombectomy efficacy) in less than approximately 1 minute; any increase in measured aspirate viscosity may be correlated to the frequency (or combination of frequencies and other setpoint-controlled system process variables) measured to be effective in aspirating thrombus.

A property of standing waves is that oscillatory translation or pressure wave amplitudes may exceed the amplitude of ordinary sound (traveling) waves in free space (i.e., not constrained by a solid body or cavity). The augmented translation or pressure amplitudes may enable embodiments of the present invention to direct augmented kinetic and acoustic energy (power) levels to (or toward) a thrombus. The thrombus may be external to the catheter, in which case the organ pipe model may apply; or the thrombus may be clogging the catheter, in which case the piano string model may apply. The length dimension of a catheter generally dictates the resonant frequencies including the fundamental (first harmonic) and overtones or higher order harmonics; the fundamental frequencies and first few higher order harmonics are shown to be in the sonic range. Therefore a sonic transmitter, transceiver, hydrophone or submersible speaker may be utilized to excite one or more frequencies thereby producing on- or off-resonance phenomena. The working lengths of catheters is variable in design and manufacturing, and the physical properties of the medium are variable, therefore the calculated values for fundamental and higher order harmonic frequencies may be considered to be a guideline. Variation about the calculated frequency may be employed in some embodiments of the present invention because accurate measurement of the relevant system process variables may not be germane to the thrombectomy procedure. Some embodiments may “sweep” imposed frequencies (of a transmitter, transceiver, submersible speaker, etc.) such that the harmonic, resonant or off-resonant frequencies are imposed irrespective of variations in dimensions or physical properties of the resonant cavity or medium. A sonic transceiver may be utilized in some embodiments to provide feedback to detect augmented medium translation or pressure amplitudes. A property of standing waves in both organ pipes and piano strings is that imposed frequencies (e.g., from a sonic transmitter, speaker, etc.) other than harmonic frequencies may excite harmonic frequencies within the system; as the imposed frequency approaches a harmonic frequency, the acoustic power, and the amplitude of medium oscillation and/or pressure waves may significantly increase. Measurement of the input power supplied to an HO may enable a thrombectomy system controller to detect resonant frequencies and/or standing wave phenomena. Alternatively, devices such as a sonic transceiver (in “listening” mode) may detect and identify natural/resonant frequencies by measuring the frequencies emitted by a resonating system. Embodiments of a thrombectomy system controller of the present invention may detect and identify natural or resonant frequencies by means including a sonic transceiver, sonic receiver or hydrophone. These experimentally determined natural or resonant frequencies may then be selected as “forcing frequencies” imparted by devices such as sonic transmitter, transceiver, transducer or underwater speaker, etc.

LF oscillator embodiments of the present invention may be intermittently or continuously operated in the frequency range of approximately 0.1 Hz (subsonic) through approximately 50 Hz (low end of sonic frequency range) to impose large amplitude oscillation/translation of fluid within a catheter; the large scale translation may act to erode, ablate, dislodge or disintegrate thrombus proximate to the tip of a catheter. The calculated maximum oscillatory fluid translation/displacement for the example was approximately 160 mm or 6.3 inches. Independently, a generally higher frequency fluid oscillation (Harmonic Oscillator) may be imparted in the sonic frequency range of up to approximately 19 kHz (the high end of the sonic frequency range). In the sonic frequency range, the translation/amplitude/displacement of a fluid/medium volume element are significantly smaller; resonance theory predicts comparatively large amplitudes at resonance (as may be observed with a piano string), however no quantification is attempted herein.

A general trend of the LF oscillator is to decrease the stroke as the frequency is increased. As an example, the LF oscillator operating at 1 Hz may have a stroke of 10 mm; at 10 Hz, the stroke may be reduced to 3 mm; at 30 Hz or greater, the stroke may be reduced to 1 mm or less, etc. An objective of an LF oscillator is to cyclically discharge and intake fluid from the distal tip of the catheter, which is preferably proximate to and directed toward thrombotic material. The thrombotic material subsequently may become a harmonic oscillator with characteristic mass (m, the mass of the thrombus), damping (b, resistance to shear) and spring constant (k, the elasticity of the thrombus or its attachment to the vessel wall). The oscillating liquid within the LF oscillator/catheter combination becomes the forcing function to drive harmonic oscillation of the thrombus such that dislodgement or disintegration may ensue. Embodiments of the LCO thrombectomy system may select multiple frequencies of the LF oscillator and harmonic oscillator (HO) such that a thrombus and surrounding tissue may be excited in one or more vibrational modes.

When a thrombus is excited at or near a natural frequency, the resulting amplitude (e.g., translational, rotational) of vibration increases, which is advantageous in dislodging or disintegrating a thrombus. Dislodging a thrombus may occur at a characteristic frequency (perhaps at or near a natural frequency of the thrombus) based upon the total mass of the thrombus and/or the shear-resistance and elasticity of any attachment to a vessel wall. Disintegrating a thrombus may occur at characteristic frequencies based upon modelling the thrombus as a collection of point masses interconnected with springs and dampers. A thrombus may therefore be dislodged at or near a first frequency and the same thrombus may be disintegrated at or near a second frequency. Selecting multiple frequencies of the LCO may excite one or more vibrational frequencies of the thrombus and its attachment mechanism to a vessel wall. Generally higher harmonic frequencies, characteristic of the system, (e.g., standing waves, fundamental frequencies, overtones, etc.) generated by a Harmonic Oscillator (HO) may be applied to augment the effects of the lower-frequency LF oscillator in some embodiments. The LF oscillator and HO may be intermittently, alternatingly, consecutively, concurrently, simultaneously or independently applied. The LF oscillator and the HO share an aspect of the present invention: oscillating a column of fluid. In the case of the LF oscillator, generally operating at lower frequencies (<approximately 50 Hz), finite fluid displacements may be imparted in the range of less than 0.5 mm to in excess of 500 mm, depending upon frequency and stroke. In the case of the HO, generally operating at higher frequencies (approximately 20 Hz to 19 kHz), the fluid displacements (with each cycle) may be considered to be in the range of 0 mm to approximately 0.5 mm. The LF oscillator and HO are complementary in some embodiments; the two oscillator systems may be distinguished from one another by finite fluid displacements vs. infinitesimal fluid displacements, and further distinguished by the HO generation of one or more standing or traveling pressure or mechanical waves within the catheter. Either the LF oscillator or the HO may generate an oscillation of a thrombus. Herein an LCO may be comprised of either or both an LF oscillator and a Harmonic Oscillator (HO), in conjunction with other system components including a catheter, pump, pressure transducer, etc. Herein LCO is a general term to describe a Liquid Column Oscillator, comprised of either/both low-frequency oscillator (LFO) or a higher-frequency Harmonic Oscillator (HO).

Objectives of an LCO thrombectomy system include the vibrational excitation of one or more natural frequencies of a system comprising thrombus, blood, saline, vasculature, attachments, catheter, etc. that result in the disintegration or dislodgement of a thrombus. It is therefore important to determine which frequencies are effective in aspirating the thrombus after disintegration or dislodgement. Some embodiments of the present invention incorporate a peristaltic pump and pressure transducer to (1) generate net aspiration suction and (2) viscometrically analyze the contents of the catheter. The references provide a detailed description of viscometric thrombectomy whereby relative mass or volume flow rate and/or % thrombus concentrations may be measured or calculated as derived quantities. In a first case, wherein thrombus is eroded by oscillatory flow or disintegrated by vibration, eroded thrombotic material is ideally entrained by the net inflow into the catheter for aspiration. Thereupon, the viscosity of the contents of the catheter may measurably increase; the LF oscillator frequency at which the viscosity increases in the catheter may be considered a preferred or desired frequency. The LF oscillator may be operated at a preferred or desired frequency until the viscosity of the aspirate returns to a lower value. Independently, an HO may be operated at one or more frequencies which may induce standing or traveling waves within the catheter at or near resonant or off-resonant frequencies which may augment, supplement or perhaps diminish the thrombectomy efficacy of the LF oscillator. This sequence may be repeated for finding multiple preferred or desired frequencies, each of which erode, ablate, dislodge, disintegrate or otherwise disrupt the thrombus in a characteristic manner. In a second case, wherein a solid bolus of thrombus is dislodged by harmonic oscillations from an LF oscillator, an HO or a combination thereof, and aspirated, the measured viscosity of the aspirate may increase to a large value which is indicative of a clogged catheter. Automated or manual countermeasures may be enacted to clear the clog.

The LF oscillator and HO are disclosed herein as a means to elicit harmonic oscillation or vibration of components including: thrombus, vessel wall, thrombus attachments, blood, saline, vasculature, catheter, etc.; the objective being that thrombus may then be aspirated by an integrated and/or independent system (e.g., suction provided by a peristaltic pump, syringe or evacuated reservoir, etc.). The vacuum level (or aspirate pump speed) and LF oscillator/HO frequencies may be factors (process variables) which may be under control of a system controller or manual input by a clinician. Additional independent systems, such as fluid column impulse mechanism, direct impingement radial or axial jets may be simultaneously or sequentially employed to further increase the effectiveness of each independent system. Embodiments of the present invention may comprise viscometry of aspirate along with a plurality of systems to perform any or all of the following: (1) apply a suction level appropriate for the measured viscosity of the aspirate, (2) excite harmonic frequencies of or within components including: thrombus, vessel wall, attachment, or catheter, (3) deploy axial and/or radial hydrodynamic jets to directly impinge upon the thrombus and/or its attachment to the vessel wall (4) employ mechanical means including obturators, rotating cutters, meshes, augers, etc., (5) deliver a fluid impulse through the catheter, and/or (6) discharge lubricious or therapeutic compounds to the thrombotic site.

Thrombectomy procedures are, by nature, experimental. The specific locations of thrombi (e.g., middle cerebral artery infarction 7 mm into segment Ml, 4 mm into popliteal artery, etc.) are typically not quantitatively known by the clinician; the clinician may therefore manipulate the thrombectomy catheter (e.g., by advancing, retracting, rotating, etc.) to position the catheter tip in proximity to a target thrombus. The clinician may be visually guided, however optical resolution and tissue radiopacity phenomena limit the clinician's ability to precisely assess (1) where the thrombus is with respect to the catheter tip, (2) what the composition of the thrombus is (e.g., soft, hard calcified, proteinaceous, fibrous, etc.) and (3) which thrombectomy operating mode (e.g., suction/vacuum level, pulse rate, rotational speed, infusion pressure, obturator manipulation, etc.) may be appreciably efficacious. The chemical and physical properties of thrombi are subject to a clinician's observation, speculation, experience and expertise. The clinician is thereby tasked with experimentally determining and detecting the location of each thrombus and subsequently enacting one or more experimental techniques (e.g., maximum, intermediate or pulsed suction/vacuum, manual manipulation of the catheter, etc.) in attempts to aspirate one or more thrombi. A successful thrombectomy procedure may be a process comprised of one or many phases (e.g., eroding a thrombus surface, disrupting attachment(s) to a vessel wall, lancing/piercing/macerating thrombus through hydrodynamic or mechanical means, vibrational excitation, etc.) that are sequentially, intermittently, continuously or randomly executed throughout any procedure.

Experimentation may be more successfully implemented when performed in conjunction with accurate and precise measurement instrumentation and meaningful experimental results. The present disclosure comprises the utilization of viscometric and/or relative flow measurements to improve the experimental results of a thrombectomy procedure by systematically conducting successive experiments (e.g., thrombectomy operating modes, suction/vacuum levels, pump speeds, etc.) and quantitatively measuring the outcome of each experiment in terms which are both meaningful and quantitative (e.g., viscosity, relative flow rate, % thrombus, % saline, etc.). In the case of a thrombectomy procedure, execution of each thrombectomy operating mode may be considered an experiment; embodiments of the present invention may comprise calculation of quantitative experimental results which may be used as feedback to provide a decision basis for subsequent experimentation. In experimental thrombectomy cases wherein the measured viscosity of aspirate (contained within the catheter) is approximately equal to that of blood (or a known blood/saline mixture), the experiment may be considered a “failure;” in cases wherein the measured viscosity is greater than that of blood, the experiment may be considered a “success.” Thus, thrombectomy operating modes determined to be “successes” may be subjected to subsequent and/or further experimentation, whereas thrombectomy operating modes determined to be “failures” may be modified or abandoned. Embodiments of the present invention comprise a plurality of thrombectomy operating modes which may be systematically executed, and whereupon the measured experimental results may be conclusively interpreted.

The present disclosure includes embodiments of thrombectomy systems and catheters which comprise either a single lumen or a plurality of lumens. Single lumen catheters are typically selected in order to minimize the overall catheter size for smaller applications, such as neurological and other applications featuring vasculature of a comparatively smaller dimension. Dual or multiple lumen catheters are typically selected for larger vasculature wherein the non-aspiration lumen(s) may be used to mechanically or hydrodynamically disrupt the thrombus. Both single and dual lumen embodiments of the present invention are disclosed herein. The present invention is applicable to both the arterial and venous sides of a mammalian cardiovascular system, and may include systems, devices and treatments for clinical indications including pulmonary embolism, arteriosclerosis, deep vein thrombosis, ischemic stroke and many others.

Following is a representative example of a sequence of (single lumen) thrombectomy operating modes which may be initiated upon viscometric (and/or visual) detection of a thrombus proximate to the catheter tip (e.g., ischemic stroke, CTO, etc.). A first (optional) step may comprise a “saline flush” wherein the catheter is filled with saline, “pushing” blood distally outward through the catheter; this step may be performed in order to fill the catheter with saline (at approximately 1 cP viscosity), rather than blood (at approximately 4 cP viscosity). The endpoint of this experimental step may be determined when the measured viscosity of the contents of the catheter is approximately that of saline. In accordance with the present teachings, an objective of a saline flush is to diminish the frictional, dissipative and/or damping effects of the fluid contained within the catheter, thereby making the catheter a more effective transmission line for hydrodynamic, fluid mechanical, oscillatory motion at sonic or subsonic frequencies, etc. as therapeutic treatment(s).

A subsequent step may be to infuse an aliquot of a lubricious and/or therapeutic fluid (e.g., glycerin, silicone oil, anticoagulant, thrombolytic agent, etc.) into the proximal end of the catheter; aliquots may be formulated to exhibit substantially greater viscosity than saline (procedural range of approximately 30 cP to greater than 1,000,000 cP for a lubricious and/or therapeutic fluid). The aliquot of lubricous and/or therapeutic fluid may be transported distally (by means of infusing saline) through the catheter to be delivered to the site of the thrombus. Thus, the aliquot may exhibit “plug flow” wherein the plug of aliquot is flanked by saline and moving distally toward the thrombus. Delivery of the aliquot to the target site may be detected volumetrically (i.e., when the viscosity of the contents of the catheter returns to approximately that of saline) or volumetrically (prescribed number of revolutions of a pump). The high-viscosity aliquot of lubricous and/or therapeutic fluid is thus delivered proximate to the thrombus to be aspirated; saline may comprise the fluid within the catheter. The endpoint of this experimental step may be determined volumetrically or when the measured viscosity of the contents of the catheter is approximately that of saline.

A subsequent step may be to induce one or more axially moving (longitudinal) pressure, velocity, momentum or impulse waveforms (or pulses) originating at the proximal end of the catheter and traversing the length of the catheter. The pressure/momentum pulse exits the distal end of the catheter and impinges upon the thrombus. The pressure/momentum pulse may be subsequently followed by aspiration to withdraw the dislodged thrombus proximally. The subsequent aspiration may be provided by means including a peristaltic pump, evacuated reservoir, LF oscillator, syringe, etc. Objectives of a single pressure/momentum pulse include to hydrodynamically force the lubricous and/or therapeutic fluid into the interstitial spaces of the thrombus and the interface (attachments) between the thrombus and surrounding vasculature. Objectives of a plurality of pressure pulses interspersed with suction include to vibrate, oscillate, rock, twist or dislodge thrombus, thereby therapeutically treating the thrombus and the interface. Objectives of oscillatory motion of fluid within the catheter include the generation of one or more resonant frequencies of systems including: vascular wall, thrombus, attachments, adhesions/cohesions, catheter, etc. Each such resonant frequency may excite a characteristic vibrational mode in the resonant system comprised of catheter, blood, saline, thrombus and vasculature.

A subsequent step may be to aspirate or withdraw the dislodged thrombus. The sequence of steps disclosed may enable any or all of the following tasks: (1) disrupting the static equilibrium of the thrombus—vasculature interface, (2) disrupting any bonding phenomena including adhesive or cohesive forces including thrombolysis, and/or (3) delivering a lubricous and/or therapeutic fluid to the interface between thrombus and vasculature. At the conclusion of any or all of these tasks, the thrombus may now be more easily aspirated by any methodology.

The phenomenon of ischemic stroke is herein modeled as a static equilibrium between a generally round (e.g., cylindrical, conical, spherical, oblate spheroid, etc.) thrombus lodged in a vessel which is considered to be generally cylindrical or conical. The thrombus may reduce, occlude or block downstream blood perfusion. Prior to the ischemic event, the blood flow rate through the affected vasculature may have been effectively normal at a characteristic linear velocity (range of approximately 1 cm/sec to in excess of 300 cm/sec), with corresponding fluid momentum. At the instant of the ischemic event, momentum forces may “push” or “compress” the thrombus into the vasculature; the vasculature may be radially expanded in the process. The vasculature thereby may exhibit radial compressive forces (from elastic pressure vessel theory) bearing upon the thrombus as surface contact forces. Subsequent to flow blockage, a differential pressure may exist between the vascular regions upstream and downstream of the stationary thrombus; this differential pressure may act to exert an axial force upon the thrombus. The forces upon the thrombus include axial pressure forces and radial compressive forces. The contact pressure at the thrombus—vasculature interface may be appreciable (range of approximately 50 Pa to 200 kPa).

A generalized methodology to dislodge and aspirate a thrombus may comprise generating an axial force on the thrombus great enough to disrupt the static equilibrium and initiate relative motion between the thrombus and vasculature. Considering a thrombus lodged in a cerebral artery, the catheter approach is typically from the “upstream” side of the thrombus wherein a distally-directed force (relative to the catheter) acting upon the thrombus may tend to translate the thrombus distally and further “tighten” the thrombus in the vasculature. Therefore, proximally directed forces (acting upon the thrombus) may be preferred for effective thrombus dislodgement and aspiration. A “pressurized” catheter may generate distally directed forces (pressure and momentum forces) acting upon fluids or solids distal to the catheter tip; the magnitude of this pressure may be limited to factors including: (1) the bore, stroke and speed of a piston pump, (2) the diameter and length of the catheter, (3) the viscosity of the fluid generally contained within the catheter. There is no physical limitation imposed upon the magnitude of the pressure observed within the catheter, the range may be from approximately 1 Pascal (1 Pa) to 10 or more megaPascals (10 MPa). A “suctioned” catheter may generate a proximally directed (low pressure) force acting upon fluids or solids proximate to the distal catheter tip. However, this low pressure force has one or more physical limitations imposed upon its magnitude; in no case may the pressure within the catheter attain a magnitude less than approximately zero absolute pressure. The lower limit of pressure within the catheter is generally physically limited by the vapor pressure of the contents of the catheter; cavitation or boiling may subsequently occur. The magnitude of proximally directed pressure forces acting upon a thrombus are physically limited to be less than approximately 1 atm. (approximately 100 kPa), multiplied by the cross sectional area of the thrombus. The magnitude of distally directed pressure and momentum forces acting upon a thrombus may not exhibit such a limitation. Hydrodynamically, a thrombus may be more effectively “pushed” deeper and more distally than a thrombus may be “pulled” proximally because of the physical limitation of low pressure. To avoid distal displacement of a thrombus, pressure (above intravascular) may be used judiciously, whereas to enable proximal displacement of a thrombus maximum attainable suction (approximately the vapor pressure of the contents of the catheter) may be desired.

The affected vasculature proximate to a thrombus may exhibit trauma or inflammation; this trauma or inflammation may initiate thrombosis or clotting at the thrombus-vasculature interface. Any such thrombosis or clotting at the interface may tend to create physicochemical bonding between the thrombus and the vasculature. The physicochemical bonds at the interface may manifest as necessitating a greater axial force to dislodge and aspirate the thrombus. The thrombus may experience (1) differential pressure forces (2) surface contact forces and (3) physicochemical bonds acting similarly to static friction at the interface. The result of clotting at the interface is that the thrombus may become increasingly more difficult to dislodge; phenomena including physicochemical bonding may manifest as an apparent increase in the observed static friction. The physicochemical bonds between the thrombus and vasculature may be broken by shearing, peeling, stretching or pulling the interface through hydrodynamic or mechanical means.

Distention of the affected vasculature may develop in the vicinity of a lodged thrombus as the thrombus is decelerated and momentum transferred into the vasculature. Vasculature is generally elastic in nature and typically undergoes cyclic expansions and contractions as result of the cardiac cycle. Objectives of embodiments of the present invention include utilization of vascular elasticity to stretch or expand slightly more than the static equilibrium configuration, thereby generating a gap between the thrombus and the vasculature. This gap may then be filled with a lubricious and/or therapeutic fluid which decreases the effective friction between the thrombus and surrounding vasculature. Certainly there is a limit to the elasticity of the vasculature before the onset of vasculature trauma. Embodiments of the present invention may cyclically oscillate the thrombus, interface and vasculature by means of oscillatory hydrodynamic forces as may be imparted by an LF oscillator or HO. Certain frequencies of oscillation may excite one or more vibrational modes of the thrombus/interface/vasculature system such that translation or rotation of thrombus with respect to the vasculature may develop. Some embodiments of the present invention may utilize an impulsive fluid shock to dislodge, disintegrate or break the static friction/physicochemical bonds between a thrombus and the surrounding vasculature. Disruption of physicochemical bonds between the thrombus and the presence of a lubricious and/or therapeutic fluid delivered to the interface may generate a configuration wherein the radial compressive forces (of the vasculature) and pressure forces dominate the static equilibrium and physicochemical bonding phenomena and static friction are minimized. This may minimize the magnitude of a proximally directed axial force required to dislodge and aspirate a thrombus.

In some elementary embodiments, the present invention may be effectively reduced to practice in systems comprising a system controller, a catheter, a peristaltic pump, a pressure transducer, and an optional saline reservoir. Operationally, the thrombectomy catheter may be manually positioned by a clinician to a thrombus; presence of thrombus may be detected by viscometric means as described herein and within the references. The catheter may be optionally flooded with saline by operating the peristaltic pump in a reverse or “negative aspiration” direction, infusing saline into the catheter, the endpoint may be determined viscometrically or volumetrically. With the catheter (optionally) primed with low-viscosity saline, the peristaltic pump may be operated in a predetermined sequence of rotations which may induce transient, impulsive or oscillatory fluid motion within the catheter. Thus, the combination of a system controller, a peristaltic pump, a pressure transducer and a catheter comprise an elementary embodiment of the present and co-pending inventions.

Commercially available peristaltic pumps are often supplied with stepper-motors wherein a single shaft rotation may be divided into a large number (range of approximately 50 to 1,000 or greater) steps per revolution and may be operable at speeds ranging between approximately 0.1 RPM to in excess of 5,000 rpm. Each revolution of the rotor of a peristaltic pump typically generates three pressure pulses per revolution, corresponding to three rollers displaced about the rotor. A representative (3-roller) peristaltic pump may transfer approximately 1.5cc of fluid per revolution or approximately 0.5cc of fluid per (ideal) one-third revolution. A (3-roller) peristaltic pump operating at approximately 600 RPM (10 Hz) may therefore deliver pressure pulses at approximately 30 Hz. However, the stepper-motor driving the peristaltic pump may be repeatedly stopped, started and reversed such that alternative, sequential, oscillatory or other unsteady flow regimes are developed within the catheter. The oscillatory, reversing or intermittent rotation of the peristaltic pump rotor may include sequences, examples of which may include: one distally directed impulse followed by a plurality of proximally directed flow cycles. The peristaltic pump, pressure transducer and catheter may then be used to viscometrically determine the presence of any thrombus aspirated by the selected thrombectomy operating mode; thus providing feedback data to assess the results of the experiment. If thrombus is present in the aspirate, the selected thrombectomy operating mode may be repeated; if no thrombus is present in the aspirate, a different, modified or altered thrombectomy operating mode may be executed and assessed for thrombectomy efficacy.

Some embodiments of the present invention feature setpoint/level control over a plurality of thrombectomy operating process variables and/or modes (e.g., aspiration rate, LF oscillator and/or HO frequency, infusion rate, catheter geometry, etc.) enables the use of standard computer programming techniques (e.g., loops and nested loops containing conditional statements) to change setpoints/levels and quantitatively measure the effect. A typical response time for a setpoint/level change may range from several milliseconds to several seconds; aspiration for viscometric measurement may require less than one second to occur (approximate range of 0.2s to 2s). In some cases, setpoints/levels (e.g., LF oscillator/HO frequency, LF oscillator stroke, infusion rate, catheter geometry, etc.) may be changed and the effects analyzed in less than one second; thus some embodiments execute approximately 60 combinations of setpoints/levels per minute, with each combination being measured for thrombectomy efficacy.

Each thrombectomy procedure may be considered an exercise comprising a sequence of experiments in thrombus removal lasting about an hour (range of approximately 5 minutes to in excess of 4 hours); within that hour-long thrombectomy procedure, a plurality (range of approximately 100 to 20,000) of experiments may be conducted upon a plurality of system process variables. Each experiment may comprise a combination of system setpoints/levels (e.g., aspiration rate, LF oscillator/HO frequency, infusion rate, etc.) that may be correlated to quantitatively measured thrombus removal. Selection of thrombectomy system setpoints/levels (or thrombectomy operating modes) of successive experiments in a thrombectomy procedure may be data driven to identify and exploit those system setpoint/level combinations which are effective at aspirating thrombus.

Some embodiments of thrombectomy systems of the present invention comprise generation of procedure data logs (i.e., log files) that document correlation between setpoints/levels (or combinations of setpoints/levels) of system process variables (factors) vs. measured thrombus extraction or thrombectomy efficacy. Procedure data log files may comprise (1) procedural data (e.g., thrombectomy operating mode vs. viscosity, local blood pressure/flow measurements, number/mass of thrombi, blood loss, etc.), (2) system configuration information (e.g., catheter length, diameter, adjustable process variables such as LF oscillator, HO, obturator, rotating cutter, hydrodynamic jet, variable geometry catheter, etc.), (3) clinical indications (e.g., ischemic stroke, deep vein thrombosis, pulmonary embolism, CTO, etc.) and (4) data pertaining to a patient, clinician, clinic, etc.

In some embodiments, thrombectomy systems of the present invention comprise any or all combinations of: (1) viscometric determination of the contents of the catheter including as a feedback mechanism for thrombectomy operating mode selection, (2) hydrodynamic erosion and/or harmonic oscillation of thrombi (and surrounding vasculature) such as may be implemented by means including an LF oscillator, (3) utilization of resonance phenomena including sonic-frequency standing and traveling waves such as may be implemented by means including an HO, and (4) auxiliary systems which may improve thrombectomy procedure outcomes by physicochemical, hydrodynamic, variable geometry, mechanical or other means.

This disclosure recites multiple embodiments, components, subsystems and/or methods including: Liquid Column Oscillator, Harmonic Oscillator, impulse mechanism, hydrodynamic lance, mechanical lance, aspiration, infusion, viscometric sampling, viscometric distance sampling, clog aversion, etc. as utilized by a generalized knowledge-based thrombectomy system or apparatus. Some embodiments of the apparatus utilize operation of subsystems of the apparatus to subject a biological system to a stimulus or deterministic event (e.g., to oscillate or aspirate fluid) and use other subsystems (e.g., viscometric sampling, time-domain viscometry, pressure measurement, flow measurement, etc.) to measure the response or effect (of the stimulus/deterministic event). This disclosure must therefore present, and provide functional descriptions of, each component or subsystem that is included herein having a function to enact deterministic events. At the conclusion of each deterministic event, other subsystems are invoked to take measurements which are reflective of the deterministic event that preceded and influenced the measurement. At the conclusion of each measurement, subsystems such as thrombectomy control flowchart are utilized by the apparatus to modify sequence and/or duration of upcoming deterministic events to be executed.

It is first objective of this disclosure to describe and define structures and operation of the subsystems that function to attrite thrombus; herein referred to as means to attrite thrombus. It is a second objective of this disclosure to describe and define structures and operation of the subsystems that function to measure thrombus, or to measure the catheter to thrombus distance; herein referred to as means to measure thrombus. Means to measure thrombus are reliant upon time-domain viscometry, as cited as references. It is a third objective of this disclosure to describe and define how to correlate the deterministic events to the thrombus measurements and to subsequently alter or modify the algorithm, taking into consideration that the algorithm selects the upcoming deterministic events to be executed. The first 16 of the initial claims, appended to this application, are devoted to defining the structures comprising the apparatus and, in some cases, the functions thereof. Each of these structures is associated with a characteristic approach to attriting thrombus, and these structures are generally adjusted to a setpoint or position before or during the course of executing a deterministic event. In some embodiments, the means to attrite thrombus are setpoint-controlled by a system controller. To bring context and examples to the claim language, the following commentary is included to provide insight.

Claim 1. An apparatus comprising:
a first fluid conduit having a proximal end and a distal end, wherein, in use of the apparatus, the distal end of the first fluid conduit is fluidically coupled to a fluid reservoir, the fluid reservoir including a material attached to a wall thereof; and a reciprocating surface, wherein the reciprocating surface displaces fluid within the first fluid conduit, and wherein the reciprocating surface is operable to create an oscillating flow of a fluid within the first fluid conduit at a frequency less than 19,000 Hz, the oscillating flow within the first fluid conduit creating an oscillating motion of the material, thereby attriting the material.

In the context of thrombectomy systems and procedures, claim 1 describes an apparatus (thrombectomy system) and a catheter (first fluid conduit) deployed within a patient vascular system (fluid reservoir) wherein a material (thrombus) is attached to a wall of the reservoir (the patient vasculature). The first fluid conduit is typically the aspiration lumen of a catheter; subsequently, additional lumens are included in the apparatus. A reciprocating surface can be a piston of LF Oscillator, diaphragm of sonic transduce or other embodiment, also herein referred to as a Liquid Column Oscillator, or LCO. In operation of the apparatus, the reciprocating surface vibrates or oscillates the fluid in the catheter, which in turn, causes the thrombus to vibrate or oscillate. The vibrating or oscillating thrombus may become loosened and/or dislodged from the vasculature, attriting and/or diminishing the size of the thrombus attached to the vasculature. Vibrational or oscillatory flow past the thrombus may erode, attrite or otherwise diminish the size of the thrombus. The eroded, attrited or dislodged thrombus may be aspirated by a catheter. Oscillating a column of fluid is included to invoke resonance phenomena (of catheter, thrombus, vasculature, etc.), however such resonance phenomena typically occur at specific frequencies. An oscillator is typically tuned to a system (like a radio is tuned to a station) whereupon the signal may be detected and amplified; it is an objective of embodiments of the invention to detect the resonant frequencies and amplify the ability to attrite thrombus. In claim 1, the oscillating flow may be at any frequency less than 19,000 Hz. In thrombectomy context, claim 1 defines the structure of an LCO and the functionality of attriting thrombus.

Claim 2. The apparatus of claim 1 including:
a transducer fluidically coupled to the fluid conduit; and a controller operable to receive input data from the transducer and vary the motion of the reciprocating surface.

In the context of thrombectomy, the transducer may be a measuring device to measure a physical property of the contents of the catheter (e.g., pressure, viscosity, relative flow rate, etc.); a controller is included to vary the operation of the reciprocating surface (e.g., frequency, stroke, amplitude, etc.). The controller receives data from the transducer and measures the amount of thrombus in the catheter. These components may be utilized to execute a sequence of cause and effect (stimulus/response) experiments in aspirating thrombus (attrited by the reciprocating surface). After each experiment, the amount of thrombus in the catheter is measured (and may be correlated to the apparatus setpoints).

Claim 3. The apparatus of claim 2 wherein varying the motion of the reciprocating surface comprises varying the speed of the motion of the reciprocating surface.

Claim 4. The apparatus of claim 2 wherein varying the motion comprises varying the distance of the motion.

In thrombectomy context, claims 3 and 4 define the variation of two operating parameters of an oscillator (LCO), frequency and stroke (or amplitude). Claim 1 defines a Liquid Column Oscillator, claim 2 includes a controller to adjust the setpoints of the LCO, claim 3 defines that the controller can adjust the speed (frequency) of the LCO and claim 4 defines that the controller can adjust the displacement or amplitude of the LCO.

claim 5. The apparatus of claim 2 including a pump fluidically coupled to the first fluid conduit.

In thrombectomy context, claim 5 includes a means to aspirate fluid through the first fluid conduit. Means to aspirate fluid is inclusive of liquid pumps, gas or vapor pumps (evacuated reservoirs), syringes, syringe pumps, hydrodynamic jets (jet pumps), valves, etc. A means to aspirate fluid is a critical component or subsystem of a thrombectomy system or apparatus to extract attrited thrombus from the patient. The means to attrite thrombus contributes to the degradation, diminution or attrition of thrombus. In thrombectomy context, the two means (to attrite and to aspirate) work in concert to attrite and aspirate thrombus. The means to aspirate fluid may be considered a means to attrite thrombus because aspiration alone is successfully used in thrombectomy systems. The inclusion of additional means to attrite thrombus is motivated by improving thrombectomy procedure efficacy.

Claim 6. The apparatus of claim 5 including:
a second fluid conduit; and a second pump fluidically coupled to the second fluid conduit.

Claim 7. The apparatus of claim 6 wherein:
the second fluid conduit is at a first location with respect to the first fluid conduit. at a first time; and the second fluid conduit is at a second location with respect to the first fluid conduit at a second time.

Claim 6 includes a second lumen in the catheter for infusion of pressurized fluid; embodiments include hydrodynamic tube, hydrodynamic lance, coiled hydrodynamic tube, etc.

These embodiments represent an additional means to attrite thrombus, generally by hydrodynamic action (e.g., jets, nozzles, etc.). Infused fluid may also represent a means to aspirate fluid within the catheter. Claim 6 introduces a means to attrite thrombus and a means to aspirate fluid by infusion and/or hydrodynamic effects. Claim 7 includes a provision that the hydrodynamic tube may be configured in multiple locations with respect to the catheter. For instance, the hydrodynamic tube may be configured to extend past the catheter tip or it may be configured to be contained fully within the catheter; each configuration being specific to the task of attriting thrombus or aspirating fluid.

Claim 8. The apparatus of claim 2 wherein the distance between the proximal end of first fluid conduit and the distal end of the first fluid conduit is a first length at the first time; and the distance between the proximal end of first fluid conduit and the distal end of the first fluid conduit is a second length at a second time.

Claim 8 enables an apparatus comprising an oscillator (e.g., LCO) to be tuned to a specific frequency by changing the length of the resonant cavity (that is the catheter). For instance, if resonant thrombus oscillation is detected at 400 Hz, the length of the catheter may be adjusted to develop a standing wave at the desired frequency of 400 Hz. Claim 8 provides an additional means to attrite thrombus by adjusting the catheter length to a desired length and corresponding desired resonant frequency. Claim 1 enables an LCO to operate at any frequency; claim 8 enables an LCO to be adjusted to a desired resonant frequency. The desired resonant frequency may be determined by means to measure thrombus.

Claim 9. The apparatus of claim 1 wherein the reciprocating surface comprises a face of a piston.

Claim 10. The apparatus of claim 1 wherein the reciprocating surface comprises a diaphragm.

claim 11. The apparatus of claim 1 wherein the reciprocating surface comprises a sonic transducer.

Claims 9, 10 and 11 recite embodiments of structures that comprise claim 1, which is construed herein as a means to attrite thrombus. Claim 11 includes a sonic transducer to the apparatus; acting as a sonic transmitter, sonic transducer functions as a means to attrite thrombus. Acting as a sonic transceiver or receiver, sonic transducers function as a means to measure thrombus. A sonic receiver may detect a resonant frequency of a thrombus; the controller may subsequently adjust the apparatus to oscillate or resonate at the detected resonant frequency of the thrombus.

claim 12. The apparatus of claim 2 wherein the transducer is operable to measure pressure.

Claim 13. The apparatus of claim 2 wherein the transducer is operable to measure frequency.

Claim 14. The apparatus of claim 2 wherein the controller is operable to measure a viscosity of the fluid within the fluid conduit.

claims 12, 13 and 14 identify the system parameters that are measured by the transducer of claim 2. Claim 12 describes the function of a pressure transducer; claim 13 describes the function of a sonic receiver. Claim 14 describes the function of an apparatus such as a time-domain viscometer (cited as reference). Claims 12, 13 and 14 further define embodiments of means to measure thrombus.

Claim 15. An apparatus comprising a fluid conduit, the fluid conduit having a proximal end and a distal end, the fluid conduit comprising:
a first lumen operable to transport a pressurized liquid from a proximal end thereof to a distal end thereof;
wherein, in use of the apparatus, the distal end of the fluid conduit is fluidically coupled to a fluid reservoir, and at least a portion of the pressurized liquid is discharged into the fluid reservoir in a radial direction, thereby generating a reaction force exerted upon the fluid conduit in a direction opposite to the radial direction, the fluid conduit thereby being forced into contact with a wall of the fluid reservoir.

claim 16. The apparatus of claim 15 wherein the conduit comprises a second lumen, wherein the distal end of the first lumen is a distance, d, from a distal end of the second lumen, wherein in use of the apparatus, the distance, d, has a first value at a first time and a second value at a second time.

In the context of thrombectomy, claim 15 describes the structure and function of a means to attrite thrombus, generally by hydrodynamic action. The structure of the fluid conduit (catheter) includes a first lumen for pressurized fluid which is transported to the distal end of the catheter, or a hydrodynamic tube. The hydrodynamic tube includes a nozzle that directs a jet of pressurized fluid into the reservoir, generally in a radial direction. The reaction force to the jet pushes the catheter in a transverse direction until it is parallel to and constrained by the vasculature; this is a preferred orientation of the catheter with respect to the vasculature. In this orientation, axial jets are parallel to the vasculature, minimizing risk of vascular trauma. In this orientation, radial jets are in position to attrite thrombus by direct impingement. Claim 16 includes functionality of moving the hydrodynamic tube proximally or distally with respect to the aspiration lumen of the catheter; thereby acting as a hydrodynamic or mechanical lance to attrite thrombus.

Claim 17. An apparatus comprising:
a) a fluid conduit having a proximal end and a distal end, wherein, in use of the apparatus, the distal end of the fluid conduit is fluidically coupled to a fluid reservoir, the fluid reservoir containing a fluid and including a material attached to a wall thereof, the material having different physical properties from the fluid;
b) a pumping system operable to generate a differential pressure between the proximal end and the distal end of the conduit, the pumping system having a setpoint and operable to create a flow of the fluid within the conduit;
c) means for attriting the material, the means having a setpoint;
d) a transducer operable to measure an amount of the material contained within the conduit;
e) a system controller, wherein the system controller:
(i) is operable to cause the means to attrite the material, receive measurement data from the transducer, adjust the setpoint of the pumping system, and adjust the setpoint of the means;
(ii) executes a first experiment comprising a combination of a first setpoint of the pumping system and a first setpoint of the means;
(iii) receives a first value of the measurement data based on the first experiment;
(iv) correlates, using the first value of the measurement data, the first experiment to the first measurement of the amount of the material contained within the catheter.

Interpreted in a thrombectomy context, claim 17 defines an apparatus that integrates a means to attrite thrombus operated in conjunction with a means to aspirate fluid and a means to measure thrombus in the aspirate; the catheter portion of these means being deployed and utilized in an environment such as within a reservoir having a material attached to the wall thereof, or a patient vascular system having a stationary thrombus. Claim 17 recites a pumping system as a specific case of a means to aspirate fluid; this is encompassing of liquid pumps, vacuum pumps/evacuated reservoirs, valves, syringes, syringe pumps, etc. The example pumping systems are stated to be setpoint controlled (e.g., number of revolutions, speed, pressure, syringe position, on/off, time, etc.). The means for attriting material may be selected from the foregoing examples cited in this disclosure, such as: LCO, HO, aspiration, infusion, hydrodynamic lance, etc.). The operational functionality of the transducer is stated to be to measure an amount of the material (thrombus) within the catheter. Example embodiments and methods may be selected from the foregoing cited examples, such as: viscometers, viscometric sampling, viscometric distance sampling, time-domain viscometry, pressure transducers, flowmeters, etc. The system controller orchestrates operation of the means to attrite thrombus with the pumping system (to aspirate thrombus) and with the transducer (to measure thrombus in the catheter). The three subsystems are utilized to execute a sequence of experiments in thrombectomy efficacy that quantify and correlate cause and effect (stimulus/response) interactions between the apparatus and the thrombus. Coordinated operation of the three subsystems enables quantification of thrombectomy efficacy for each experiment and to rank (or sort) efficacious setpoints for means to attrite and aspirate thrombus. Moreover, the system controller is operable to adapt the sequence (of experiments in thrombectomy efficacy) such that efficacious setpoints of the apparatus are operated longer or more often (e.g., with increased duration or repetitiously). Claim 17 recites: a means to attrite thrombus, a pumping system (a means to aspirate fluid) and a transducer (a means to measure thrombus); this anticipates a plurality of means to attrite thrombus (LCO, HO, infusion, hydrodynamic lance, etc.) wherein a large number of subsystems and setpoints (factors and levels) are available for experimentation (in thrombectomy efficacy). Claim 17 describes the apparatus and operations for conducting an experiment in thrombectomy efficacy and then correlating the measurement data to the setpoints prescribed by the system controller (thereby defining a deterministic event). The apparatus of claim 17 thereby: executes a deterministic event, measures the effect of the deterministic event and correlates the apparatus setpoint(s) to the measured effect of the deterministic event. Subsequently, those apparatus setpoint(s) correlating to thrombectomy efficacy may be repeated, extended or otherwise further explored experimentally.

Claim 18. A method comprising:
(i) performing a first thrombectomy procedure using a thrombectomy apparatus, wherein the thrombectomy apparatus includes setpoint-controlled means to attrite thrombus, setpoint-controlled means to aspirate a fluid within a catheter, and means to measure thrombus within the catheter, wherein the first thrombectomy procedure comprises operating the thrombectomy apparatus at successive setpoints, wherein for each successive set point, an amount of thrombus within the catheter is measured and correlated to the respective setpoint, and wherein some of the setpoints positively correlate to the amount of thrombus within the catheter; and
(ii) performing a second thrombectomy procedure using the thrombectomy apparatus, wherein at least some of the setpoints that positively correlate to the amount of thrombus within the catheter are used to as setpoints for the setpoint-controlled means to attrite thrombus and the setpoint-controlled means to aspirate a fluid within a catheter.

claim 18 illustrates dynamic adaptation of a thrombectomy control algorithm to conduct a predetermined sequence of experiments and repeat, interrupt or otherwise alter the sequence based upon detection of positive correlation of experiment(s) to thrombectomy efficacy. In a first limiting case, the first and second thrombectomy procedures may be successive or sequential thrombectomy operating modes (experiments) conducted upon a single patient during a single session. In a second limiting case, the first and second thrombectomy procedures are conducted at different times, upon different patients, with different clinicians and equipment, etc. In both limiting cases, the second procedure utilizes data collected during the first procedure, albeit by differing means. In the case where the second procedure is an immediate continuation of the first procedure, the correlation data (of the first procedure) are used to identify efficacious experiments so that the identified experiments may be repeated or altered. For instance, if the first experiment positively correlates to thrombus, then the second experiment may be to repeat the first experiment. Each experiment is conducted to correlate apparatus setpoints to thrombectomy efficacy; the objective of a thrombectomy procedure is to aspirate thrombus. Therefore if any experiment positively correlates to aspirating thrombus, then repeating or extending the duration of that experiment contributes to the objective of aspirating thrombus.

claim 18 also illustrates a distinction in utilization of intra-procedural and inter-procedural (or post-procedural) data, while encompassing both. The first procedure cannot utilize or benefit from data that has not yet been collected (by experiments not yet performed); however, the second procedure can utilize and benefit from data that was collected during the first procedure. Therefore, the second procedure benefits from access to a greater knowledge base than the first procedure. In the limiting case where the first and second procedures are sequentially executed in a single session, intra-procedural data are available for the thrombectomy control algorithm to adapt the sequence of apparatus setpoints (e.g., such as duration, order, repetition, etc.). In the limiting case where the first and second procedures are (spatially and temporally) independent of one another, compiled data from a plurality of “first procedures” may be used to develop improved thrombectomy control algorithms, initial values, threshold values, experiment sequences, etc. The second thrombectomy procedure may benefit from updated software/firmware having been installed in the interim period between the first and second procedures. In this manner, each experiment that is conducted, in each thrombectomy procedure that is performed, contributes to a knowledge base which correlates apparatus setpoints to thrombectomy efficacy. Each thrombectomy procedure thereby becomes an extension of all prior procedures, benefitting from historical data and contributing to a knowledge-base to be utilized in future procedures.

### BRIEF DESCRIPTION OF THE DRAWINGS

FIG. 1A depicts a Liquid Column Oscillator (LCO)/Harmonic Oscillator (HO) catheter combination positioned within a blood vessel. The piston is to the right, and the thrombus shown pushed to the right. The acoustic tube is shorter and there is a higher frequency standing wave emanating from the catheter.

FIG. 1B depicts an LCO/HO catheter combination with the piston to the left, and the thrombus shown pulled to the left. The acoustic tube is longer and there is a lower frequency standing wave emanating from the catheter.

FIG. 1C depicts pressure limiter 10 in exploded view.

FIG. 1D depicts pressure limiter 10 in assembled view under pressure.

FIG. 1E depicts pressure limiter 10 under atmospheric pressure or vacuum/suction.

FIG. 1F depicts pressure limiter 10 under pressure and venting to exhaust port/tube.

FIG. 1G depicts impulse mechanism 90 is the cocked state.

FIG. 1H depicts impulse mechanism 90 in the uncocked state.

FIG. 2A depicts a thrombus in a neutral configuration within a blood vessel.

FIG. 2B depicts a thrombus in a distal/extended configuration within a blood vessel.

FIG. 2C depicts a thrombus in a neutral configuration within a blood vessel.

FIG. 2D depicts a thrombus in a proximal/retracted configuration within a blood vessel.

FIG. 2E depicts an oscillating vessel wall; the vessel wall is shown distended in a first vibrational mode.

FIG. 2F depicts the vessel wall in a first undulating vibrational mode.

FIG. 2G depicts the vessel wall in a second undulating vibrational mode.

FIG. 3A depicts an LCO 100 with catheter tip a distance away from a thrombus.

FIG. 3B depicts a catheter tip in close proximity to a thrombus.

FIG. 3C depicts a catheter tip penetrating a thrombus.

FIG. 3D depicts a catheter tip and thrombus bolus; the catheter tip has been withdrawn from the thrombus.

FIG. 4A depicts oscillating flow pressure vs time waveforms for three combinations of LF oscillator frequency and stroke.

FIG. 4B depicts a decaying oscillating flow pressure waveform vs distance for three combinations of LF oscillator frequency and stroke.

FIG. 4C depicts an effect of viscosity on the oscillating pressure waveform.

FIG. 4D depicts a velocity waveform of oscillating flow with superposition of aspiration. With aspiration, the net flow out of the catheter may be positive, negative or zero.

FIG. 4E depicts a pressure vs time waveform for a pressure-limited LF oscillator.

FIG. 4F depicts a pressure vs time waveform for a time-varying piston stroke cycle.

FIG. 5A depicts an LCO thrombectomy system with viscometric aspiration and infusion 400.

FIG. 5B depicts an LCO thrombectomy system with viscometric aspiration and infusion 400 featuring mechanized positioning of infusion tube within the catheter.

FIG. 5C depicts the distal ends of catheter and infusion tube showing axial and radial nozzles.

FIG. 5D depicts the distal end of infusion tube proximal to a thrombus.

FIG. 5E depicts the distal end of infusion tube extended to hydrodynamically interact with the thrombus.

FIG. 6 depicts measured aspirate viscosity vs. time during (a portion of) a representative thrombectomy procedure. Phases of thrombus aspiration are enumerated and distinguished from blood.

FIG. 7 depicts seven representative thrombectomy system process variables (factors) which may be setpoint/level-controlled. The units, range, increment, number of increments and loop termination index are also shown.

FIG. 8 depicts a representative system controller and the interfaces to input data and output system control.

FIG. 9A depicts a representative thrombectomy control flowchart.

FIG. 9B depicts a representative “effect” subroutine to evaluate current data with respect to previously determined values and execute control actions based upon the evaluations.

FIG. 9C depicts a representative “distance” subroutine that measures the distance between a catheter tip and a thrombus, and to execute control actions based upon the measurement.

FIG. 9D depicts a representative “clog detect/avert” subroutine that operates proactive measures to prevent clogging of a catheter.

FIG. 10 depicts a representative two-factor response surface, wherein frequency and stroke are independent variables and aspirate viscosity is the dependent variable.

FIG. 11 depicts a representative three-factor response surface wherein factor (1), factor (2) and factor (3) are independent variables; aspirate viscosity is the dependent variable. Four types of thrombus (anatomical/thrombus morphological) are shown as response surfaces/volumes.

FIG. 12 depicts a plurality of knowledge-based thrombectomy systems, each performing a plurality of thrombectomy procedures; a plurality of log data files are shown within a system and distributed throughout a network of systems.

FIG. 13A depicts page 1 of a representative thrombectomy data log file.

FIG. 13B depicts page 2 of the representative thrombectomy data log file.

FIG. 13C depicts page 3 of the representative thrombectomy data log file.

FIG. 14 depicts a block diagram depicting a physical relationship between a thrombectomy system, the patient vascular system, the surgical suite and remote database/compiler.

FIG. 15 depicts a representative architecture and communication pathways between a system controller and a database/compiler.

### DETAILED DESCRIPTION OF THE DRAWINGS

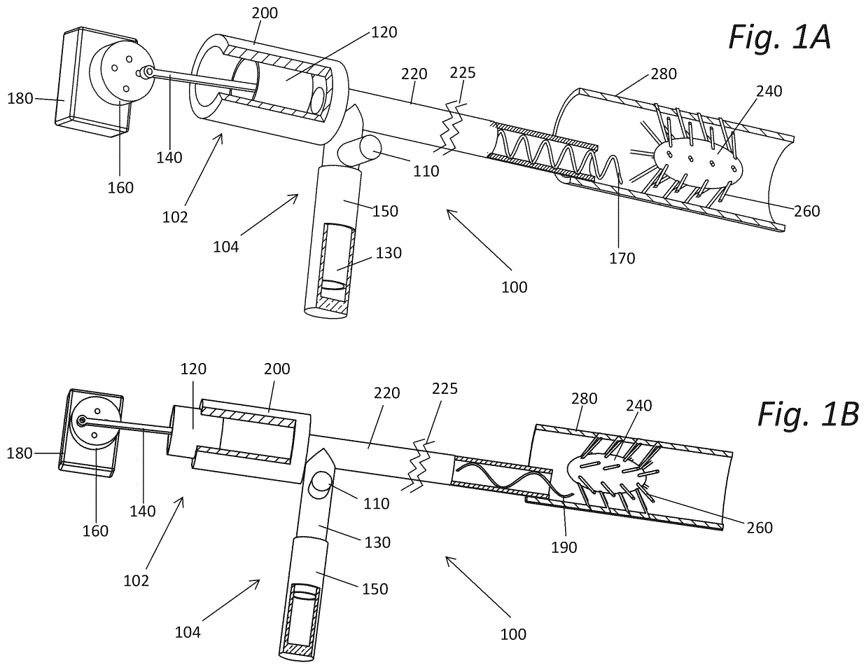

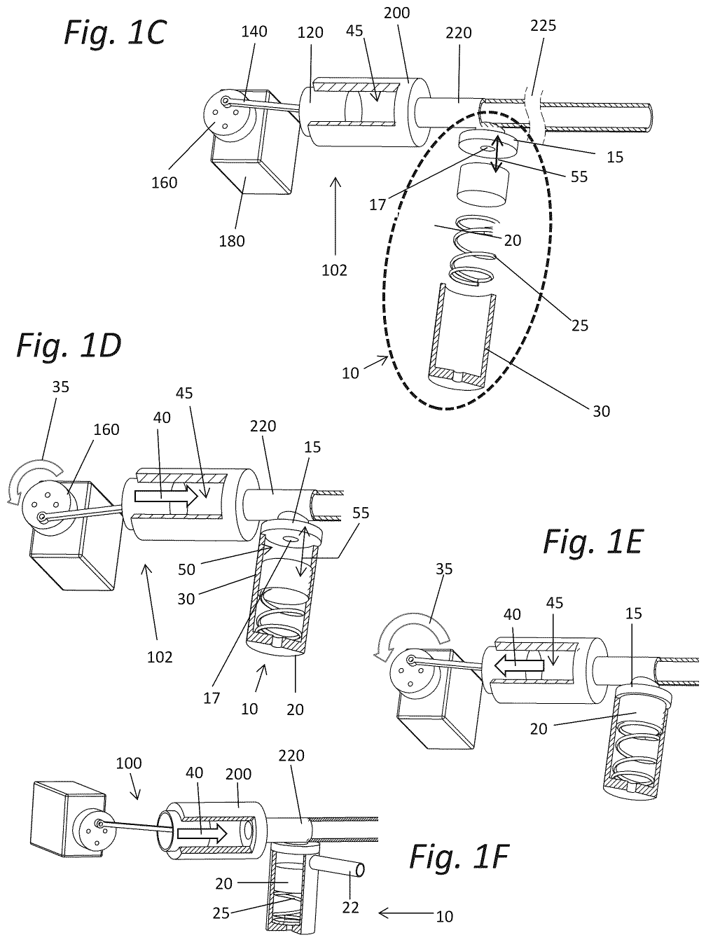

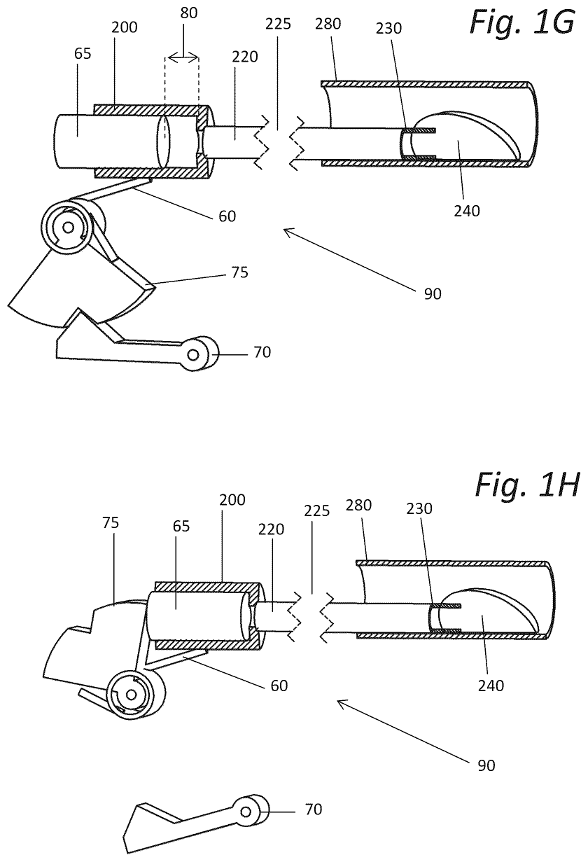

Liquid Column Oscillator 100. FIGS. 1A and 1B depict liquid column oscillator (“LCO”) 100 fluidically coupled to a portion of a patient's vascular system containing thrombus 240. LCO 100 includes two independently operating oscillator systems. Namely, (i) lower-frequency (LF) oscillator 102 including piston 120, cylinder 200, and associated actuation elements, and (ii) higher-frequency, or harmonic (HO) oscillator 104, including sonic transducer 110, acoustic tube 130, and slide 150. The two oscillator systems are fluidically coupled to catheter 220, which, in use, is inserted into a portion of the vasculature shown as vascular access 225. Vascular Access 225 is typically gained by means of a medical device such as an introducer sheath (not shown). LCO 100 (and subsequently presented embodiments) is representative of an apparatus that features multiple subsystems that are setpoint controlled. As previously discussed, a thrombectomy featuring a plurality of subsystems (factors), each having a plurality of setpoints (levels), may give rise to a large number of combinations thereof.

Among objectives of some embodiments (such as LCO 100) is to conduct a sequence of experiments. Some experiments enable a correlation of cause and effect (or stimulus-response) phenomena of a system (e.g., a biological system such as: vascular system, lymph system, gastric system, etc.) to deterministic events (e.g., causes, stimuli, etc.). During these experiments, the apparatus may be termed “in operation” or “operated,” when systems of the apparatus (e.g., motors, oscillators, pumps, etc.) are in operation (e.g., rotating, oscillating, pumping, etc.). A typical sequence of events comprising an experiment is presented as an example. Step1: the apparatus setpoint(s) is/are assigned. Step 2: the apparatus is operated with the setpoints assigned in step 1. Step 3: a property of the aspirate is measured. This concludes the experiment; step 4 may be optionally conducted concomitantly with, or subsequent to, the experiment. Step 4: the assigned apparatus setpoint(s) are correlated to the measured property of the aspirate. Embodiments of the invention provide a way to correlate measurement data (e.g., viscosity, relative/absolute flow rate, % thrombus, thrombus load, flow characteristic, state of flow, etc.) to deterministic events (e.g., setpoints, apparatus operation, manipulation, repositioning, etc.); the measurement data being predicated upon the deterministic events. Some depicted embodiments illustrate a means, apparatus or structure that enables cause and effect or stimulus-response phenomena to be measured, documented and/or analyzed.

In operation, LCO 100 generates oscillatory motion and/or pressure fluctuations of the contents of catheter 220 (e.g., blood, saline, thrombus, etc.) and that of tissue proximate to the end of the catheter. Among any other functionality, the oscillatory flow and/or pressure generated by LCO 100 dislodges, disintegrates, or partially attrites thrombus 240/attachments 260 that are proximate to the distal end of the catheter in the vasculature. In operation, LCO 100 generates oscillatory motion and/or pressure fluctuations of the contents of catheter 220 (e.g., blood, saline, thrombus, etc.) and that of tissue proximate to the end of the catheter. Among any other functionality, the oscillatory flow and/or pressure generated by LCO 100 dislodges, disintegrates, or partially attrites thrombus 240/attachments 260 that are proximate to the distal end of the catheter in the vasculature. In operation, LCO 100 generates oscillatory motion and/or pressure fluctuations of the contents of catheter 220 (e.g., blood, saline, thrombus, etc.) and that of tissue proximate to the end of the catheter. Among any other functionality, the oscillatory flow and/or pressure generated by LCO 100 dislodges, disintegrates, or partially attrites thrombus 240/attachments 260 that are proximate to the distal end of the catheter in the vasculature. Thrombus 240 is shown connected to vessel wall 280 by attachments 260. Attachments 260 may be fibers, tissue, thrombotic or other material; they are depicted to represent the physical connections between thrombus 240 and vessel wall 280. As discussed further below, the two oscillator systems (LF oscillator 102 and harmonic oscillator (HO) 104) are independently controlled such that multiple frequencies of oscillatory flow and/or pressure may be generated, to various ends. Some embodiments of LCO 100 also include aspiration and/or infusion, and/or other systems, which are omitted in FIG. 1A and FIG. 1B for clarity.

Relatively Lower-Frequency (LF) Oscillator 102. In the illustrative embodiment depicted in FIGS. 1A and 1B, LF oscillator 102 includes piston 120, cylinder 200, and several actuation elements. In the illustrative embodiment, the actuation elements include motor 180, crankshaft 160, and connecting rod 140. Piston 120, which is disposed within cylinder 200 (shown in cutaway), is coupled to connecting rod 140. Motor 180 rotates crankshaft 160 and connecting rod 140, which, in turn, drives piston 120 to reciprocating movement. Fluid within catheter 220 thereby undergoes oscillatory motion in the axial direction, resulting in repetitive discharge and intake of fluid through distal end of catheter 220.

FIG. 1A depicts piston 120 at or near Top Dead Center (TDC) such that fluid (typically blood, saline and/or thrombus within catheter 220) has been pushed outward, thus impinging upon thrombus 240. This outward flow pushes thrombus 240 distally; attachments 260 are shown being stretched distally (away from LCO 100).

At some frequencies and amplitudes (i.e., strokes), the sequential intake and discharge of fluid in the vicinity of thrombus 240 and attachments 260 erodes thrombus 240; this effect may be viscometrically detected in the aspirate. At some frequencies, thrombus 240 undergoes harmonic oscillatory motion, thereby putting alternating stresses on attachments 260. These alternating stresses tend to disrupt fibrous networks within thrombus 240 or attachments 260. At some frequencies, the amplitude of oscillation of thrombus 240 and attachments 260 tend to dislodge or disintegrate thrombus 240/attachments 260. At some frequencies, vessel wall 280 is excited to one or more vibrational modes that cause repetitive increases and decreases in the diameter of vessel wall 280. These vibrational modes augment the stresses within thrombus 240 and attachments 260. Piston 120 and cylinder 220 are operable at variable frequencies (i.e., by changing the speed of motor 180) and variable stroke (crankshaft 160 has multiple holes for stroke selection). Piston 120 typically reciprocates at subsonic (≈0.1 Hz to ≈20 Hz) to low sonic (≈20 Hz to ≈50 Hz) frequencies, with stroke appropriate for the frequency and desired pressure or flow amplitude.

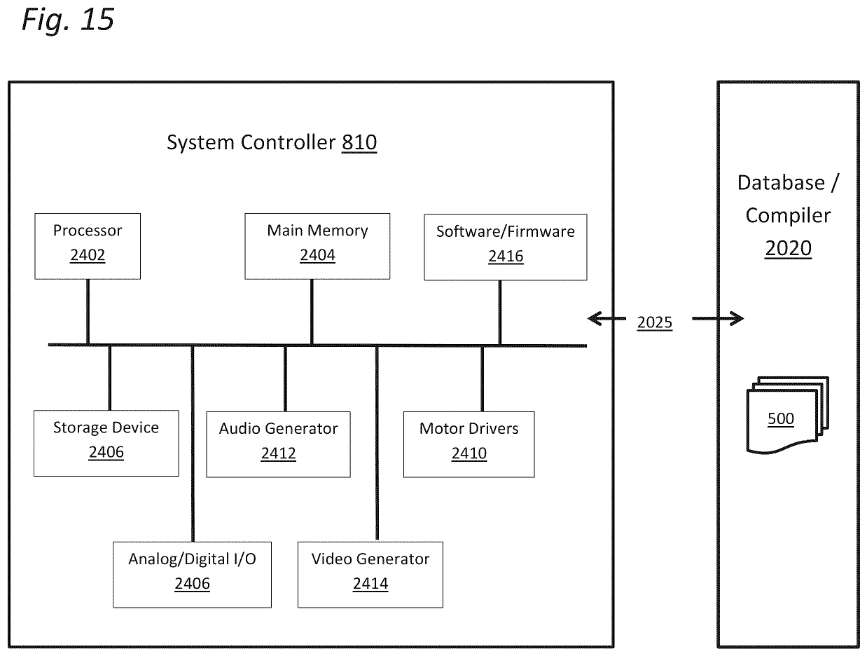

Relatively Higher-Frequency Harmonic Oscillator System (HO) 104. LCO 100 shown in FIG. 1A also depicts a second oscillator system, harmonic oscillator (HO) 104, including sonic transducer 110, acoustic tube 130 and slide 150. Slide 150, depicted in cutaway, is shown in an upward position in FIG. 1A such that acoustic tube 130 is effectively shortened. Sonic transducer 110 operates at low-sonic (≈20 Hz) to sonic (up to ≈19 kHz) frequencies (i.e., audible frequencies), thus operating at higher frequencies and lower amplitudes than piston 120. Sonic transducer 110 generates one or more standing or traveling waves at one or more frequencies; high frequency standing wave 170 is shown within and emanating from catheter 220. In various embodiments, sonic transducer 110 is a sonic transmitter, a sonic receiver, a sonic transceiver, a hydrophone, a submersible speaker, or the like. For embodiments in which sonic transducer 110 is a sonic transceiver, hydrophone or analogue, the system controller (described in conjunction with FIG. 15) may be operated to intermittently transmit and receive signals in the sonic or audible frequency range.

The vibrating thrombus 240, attachment 260, catheter 220 and/or vessel wall 280 may emit (as well as absorb) at sonic or subsonic frequencies. In some embodiments, LCO 100 includes a “listening” mode wherein sonic transducer 110 detects the aforementioned sonic or subsonic frequency emissions to determine vibrational frequencies and/or amplitudes of the system vibrational characteristics.

FIG. 1B depicts LCO 100 in a different operational configuration in which: (1) piston 120 is at or near Bottom Dead Center (BDC) and (2) acoustic tube 130 is lengthened by moving slide 150 in the downward direction, effectively lengthening acoustic tube 130. In some other embodiments, rollers or pinch valves are used to shorten and lengthen acoustic tube 130. In FIG. 1B, piston 120 is shown at or near BDC such that fluid, generally within catheter 220, has been drawn inward, creating suction that draws thrombus 240 proximally. Attachment 260 is shown stretched proximally in FIG. 1B.

In FIG. 1B, slide 150 has been moved downward to increase the length of acoustic tube 130, thereby decreasing the frequencies of any standing or traveling waves existent within catheter 220. Low frequency standing wave 190 corresponds to the longer length of acoustic tube 130. In some embodiments, sonic transducer 110 is operated at one or more frequencies that produce standing waves and overtones (n^{th} harmonics). Some embodiments of the present invention exclude acoustic tube 130 (or a variable-length embodiment thereof), such that fewer or different natural frequencies are generated. Some other embodiments exclude sonic transducer 110, and yet some additional embodiments exclude both sonic transducer 110 and acoustic tube 130. And some additional embodiments include one or both of sonic transducer 110 and acoustic tube 130, but exclude LF oscillator system 102.

In FIG. 1A and FIG. 1B, two modes of liquid column oscillation and or pressure wave transmission are shown as a single embodiment. LF oscillator 102, shown comprised of piston 120 and cylinder 200, typically operates at relatively lower frequencies and relatively larger (fluid displacement) amplitude. Harmonic oscillator (HO) 104, shown comprised of sonic transducer 110, typically operates at relatively higher frequencies and relatively smaller (fluid displacement) amplitudes. Large-scale movements of thrombus 240 with respect to vessel wall 280 tend occur at lower frequencies. The scale of these movements is expected to be in a range of about 1% to about 50% of a characteristic dimension (e.g., length, diameter, gap, thickness, etc.) of thrombus 240. Such large-scale movements of thrombus 240 tend to stretch, disrupt, break, or disintegrate attachment 260. Small-scale, internal movements of thrombus 240 and attachment 260 occur at relatively higher frequencies and smaller amplitudes. The scale of these movements is expected to range from about 1 nanometer (1 nm) to approximately 5% of a characteristic dimension. These small-scale movements tend to disrupt the structural integrity of both thrombus 240 and attachment 260. The dislodged, disintegrated, or partially disintegrated thrombus 240/attachments 260 are then aspirated.

Pressure Limiter 10. FIGS. 1C through 1F depict pressure limiter 10 being used in conjunction with LF Oscillator 102. FIG. 1C depicts an exploded/cutaway view of pressure limiter 10, the salient elements of which include: orifice plate 15, pressure limiter piston 20, pressure limiter spring 25, and pressure limiter cylinder 30. Pressure limiter displacement 55 is the distance between pressure limiter piston 20 and orifice plate 15. In the illustrative embodiment, pressure limiter 10 is fluidically coupled to LF oscillator 102 through catheter 220. However, in some other embodiments, pressure limiter 10 is coupled to LF Oscillator 102 via cylinder 200, these two fluid pathways being functionally equivalent.

Pressure limiter 10 operates by transferring fluid through orifice 17 of orifice plate 15, displacing pressure limiter piston 20 and compressing pressure limiter spring 25. Pressure limiter 10 limits the maximum operational positive pressure (i.e., greater than atmospheric or intravascular pressure) within cylinder 200, but does not limit the extent to which negative pressure (i.e., less than atmospheric or intravascular pressure) is developed. Limiting positive cylinder pressure 45 prevents undesirable outflow of aspirate (or other fluid) through catheter 220. Clinical indications (e.g., antegrade or retrograde native blood flow, risk of embolic release, etc.), might determine the necessity to regulate pressure via pressure limiter 10. Some embodiments of pressure limiter 10 include structures such as a flexible diaphragm, rather than the depicted piston and spring. The pressure limiting “setpoint” of pressure limiter 10 may be factory or field calibrated or adjusted by, for example, compressing the preload of pressure limiter spring 25. This is accomplished, for example, via a threaded connection between pressure limiter cylinder 30 and orifice plate 15, or an adjustment knob. In some embodiments, orifice 17 is sized to be large enough that flow is unimpeded into (and out of) pressure limiter 10, such that the volume of aspirate outflow (through catheter 220) is diminished or eliminated. Such sizing may be determined by simple experimentation.

FIG. 1D depicts pressure limiter 10 in operation to limit cylinder pressure 45 in LF oscillator 102. FIG. 1D depicts the compression stroke of LF Oscillator 102. Crankshaft rotation direction 35 depicts counterclockwise rotation of crankshaft 160 such that piston 120 is traveling (to the right) in piston direction 40, thereby increasing cylinder pressure 45. Fluid displaced by the rotation of crankshaft 160 has two potential pathways: (1) through catheter 220 (and into the patient blood stream) and/or (2) through orifice plate 15 and into pressure limiter cylinder 30, thereby increasing pressure limiter pressure 50. As pressure limiter pressure 50 increases, pressure limiter spring 25 becomes compressed as pressure limiter piston 20 is forced “downward” in FIG. 1D. Neglecting friction between pressure limiter piston 20 and pressure limiter cylinder 30, the magnitude of pressure limiter pressure 50 may be estimated by Hooke's Law, P=kx/A, where P is pressure limiter pressure 50, k is the spring constant of pressure limiter spring 25, A is the area of pressure limiter piston 20, and x is pressure limiter displacement 55, measured from the free length of pressure limiter spring 25.

FIG. 1E depicts pressure limiter 10 in operation during the intake or suction stroke of LF oscillator 102. Pressure limiter piston 20 is shown in contact with orifice plate 15 such that orifice 17 (not visible in FIG. 1E) is occluded. Piston direction 40 is shown to be to the left in FIG. 1E, and cylinder pressure 45 is generally decreasing throughout the intake or suction stroke. In the event of cavitation or boiling, cylinder pressure 45 may remain approximately constant at approximately the vapor pressure of aspirate, thus achieving maximum vacuum or suction levels (minimum absolute pressure). On the successive (compression) stroke of LF Oscillator 102, cylinder pressure 45 may remain approximately constant (at approximately the vapor pressure) until the vaporized aspirate has condensed to the liquid phase, at which time cylinder pressure 45 may increase.

Eq. 1 through eq. 8 provide mathematical means to estimate the position, amplitude, velocity, acceleration, and pressure of a finite fluid volume of aspirate within a cylinder 200. Example calculations are provided for given dimensions and at constant operating speed (e.g., Hz, RPM, etc.) for an ideal, massless, inviscid fluid as working fluid or aspirate. In practice, fluid viscosity may limit the fluid velocity and acceleration that develops. As discussed above, some embodiments of LF oscillator 102 include a mechanism to limit maximum pressure, such as pressure limiter 10. Additionally, maximum cylinder pressure 45 may be limited by varying the angular velocity of crankshaft 160 such that the speed of piston 120 is “faster” on the intake or suction stroke and “slower” on the successive compression stroke. In this manner, full vacuum (e.g., minimum absolute pressure, cavitation, boiling or vapor pressure, etc.) may be developed during the intake or suction stroke without incurring excessive pressure (and undesirable aspirate outflow) on the compression stroke.

FIG. 1F depicts an embodiment of pressure limiter 10 that includes exhaust port/tube 22. Pressure limiter piston 20 is depicted displaced downward, compressing pressure limiter spring 25, thereby opening exhaust port/tube 22 (shown open to atmosphere for clarity). Embodiments of pressure limiter 10 with exhaust port/tube 22 include a tube that conveys exhausted fluid to a reservoir or vessel, such as a drain or waste reservoir (not shown). FIG. 1F depicts (LF oscillator 102) piston direction 40 to be rightward, thereby increasing pressure in cylinder 200, catheter 220 and pressure limiter 10. In FIG. 1E, pressure limiter piston 20 is depicted in the upward configuration and thereby closing, occluding or “blocking off” orifice 17; in FIG. 1F, pressure limiter piston 20 is depicted displaced downwardly, opening both orifice 17 and exhaust port/tube 22. In FIG. 1F, pressurized fluid generally contained within cylinder 200 and catheter 220 flows through orifice 17 and exhaust port/tube 22 to a drain or waste reservoir. The embodiment of FIG. 1F thereby limits pressure generally within catheter 220 to a fixed or adjustable pressure level to a value that is above ambient, atmospheric, or intravascular pressure. This not only limits the magnitude of pressure generally within catheter 220 but also limits, reduces, or diminishes the volume (or mass) of fluid discharged from the distal end of catheter 220 during a compression stroke of LF oscillator 102. Reducing, etc., the volume (or mass) of fluid discharged from the distal end of catheter 220 is often desirable in thrombectomy procedures because downstream vasculature may have decreased diameter that such that a thrombus may be “wedged” therein due to the flow of discharged fluid.

Impulse Mechanism 90. FIGS. 1G and 1H depict impulse mechanism 90, which is an alternative to LF oscillator 102 for disrupting or dislodging thrombus 240. It does so by imparting a high pressure, short-duration pressure pulse or and/or travelling wave through catheter 220. Impulse mechanism 90 includes impulse piston 65, cylinder 200, sear 70, hammer 75, and torsion spring 60. Cylinder 200 couples to catheter 220. Vascular access 225 delineates intravascular from extracorporeal portions of catheter 220.

In FIG. 1G, impulse mechanism 90 is depicted in the “cocked” or ready configuration. Impulse piston 65 is slidingly engaged within cylinder 200, shown in cutaway view to illustrate impulse piston distance 80. Hammer 75 is shown rotated counterclockwise such that torsion spring 60 is stressed; sear 70 engages in a notch in hammer 75 akin to a firearm hammer/sear assembly.

FIG. 1H depicts impulse mechanism in the “uncocked” or “fired” configuration. Impulse piston distance (referenced as “80” in FIG. 1G) is reduced to effectively zero by the rightward movement of impulse piston 65, displacing fluid distally through catheter 220. In FIG. 1G and FIG. 1H, catheter tip 230 is shown to have penetrated thrombus 240. FIG. 1H does not show any change to the shape of thrombus 240, nor to the interface to catheter tip 230 because any such illustration is speculative. Methodologies of the present invention may include immediate viscometric/flow analysis to assess the thrombectomy efficacy of the fluid impulse imparted by impulse mechanism 90.

Impulse mechanism 90 imparts a high-pressure, short-duration pressure pulse or and/or travelling wave through catheter 220. The volume of the displaced fluid is approximated by the impulse piston area multiplied by impulse piston distance 80. Typically, the displaced volume is in the range of about 0.01cc to about 5cc. Some embodiments limit the displaced volume to less than approximately 1cc such that undesirable phenomena, including excessive embolization and wedging of a thrombus into a smaller vascular region may be avoided. Impulse mechanism 90 is intended to disrupt or dislodge thrombus 240, but without fragmentation that might release emboli. Impulse mechanism 90 may also be used to clear a clogged or corked catheter 220.

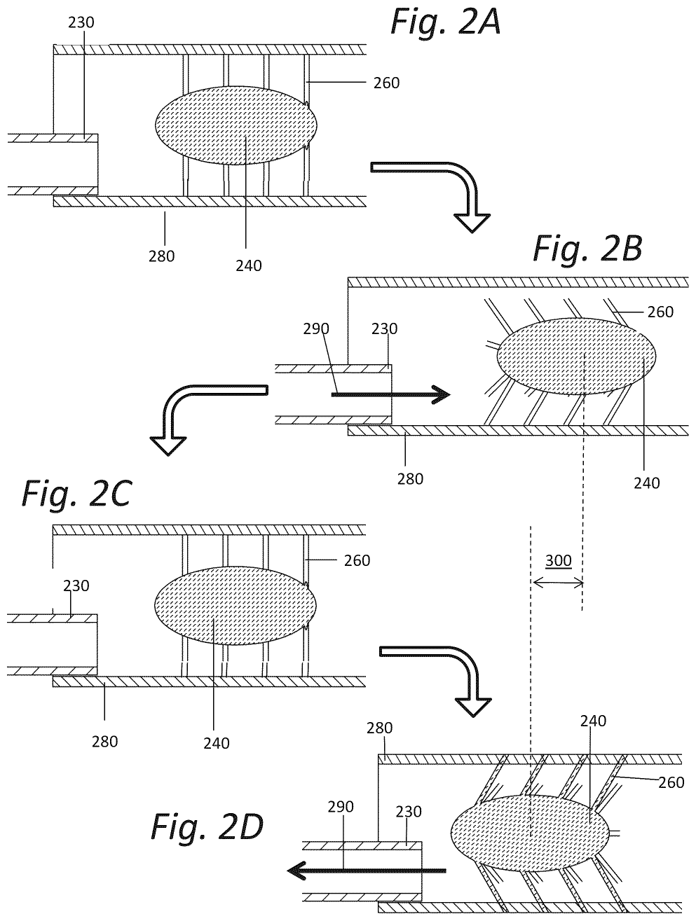

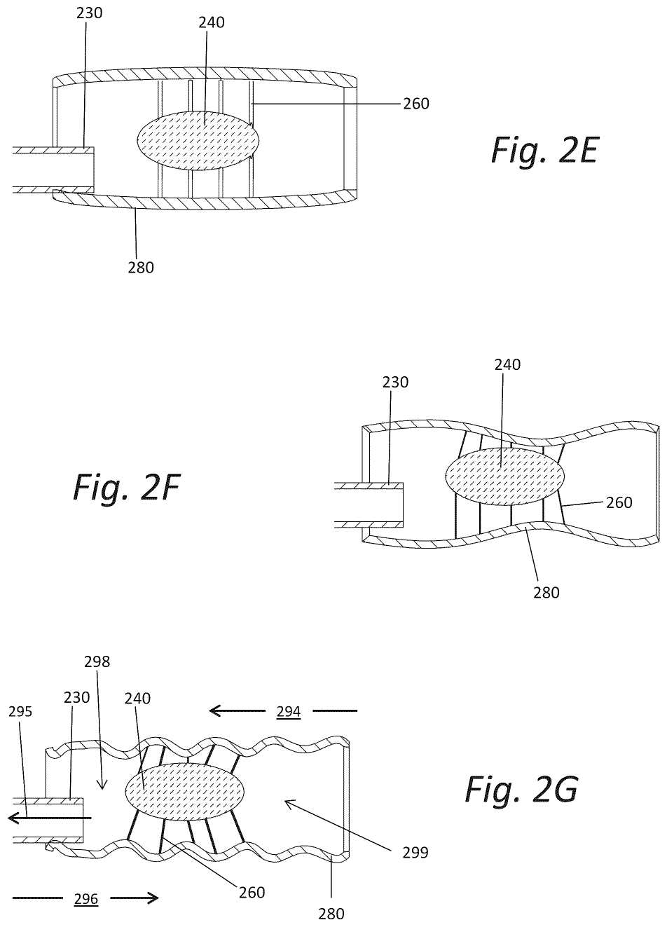

FIG. 2A through FIG. 2G depict large-scale oscillatory effects of LF oscillator 102 to disrupt, dislodge, or disintegrate thrombus 240 and/or attachments 260. FIGS. 2A through 2D depict a time sequence of thrombus 240 undergoing axial oscillation of amplitude 300 within a blood vessel wall 280. FIGS. 2A through 2D illustrate large scale oscillations of thrombus 240. These large-scale oscillations typically occur at relatively low frequency operational range of the present invention (e.g., approximately 0.1 Hz to 50 Hz).

FIG. 2A depicts thrombus 240 in a quiescent state and connected to vessel wall 280 by multiple attachments 260. Attachments 260 are shown vertical and unstressed. In the absence of any interventional device, such as a thrombectomy catheter, FIG. 2A represents the thrombus as it exists (generally in static equilibrium) in an untreated patient. Attachments 260 are shown to be symmetrically emanating in multiple radial directions from thrombus 240; clinically, thrombus 240 may exhibit significant asymmetry within vessel wall 280, and attachments 260 may exist in a portion of the perimeter, boundary, or circumference of vessel wall 280.

FIG. 2B depicts oscillatory flow 290 discharging (in the distal direction) from catheter tip 230 and directed to impinge upon thrombus 240 and/or attachment 260. Forces, including momentum, viscous drag, differential pressure, pressure drag and/erosive forces, are shown to stretch attachments 260 and translate thrombus 240 in the distal direction to generate the configuration shown in FIG. 2B. Oscillatory flow 290 is approximately equal to the sum of flow produced by LF oscillator 102 and any supplemental flow (e.g., aspiration, infusion, etc.) that may simultaneously exist.

In FIG. 2C, after oscillatory flow 290 (not shown, having an instantaneous zero velocity) from catheter tip 230 has subsided, thrombus 240 has returned to a neutral position and attachments 260 have relaxed to an unstressed configuration. For clarity, thrombus 240 and piston 120 are depicted “in phase” with one another (i.e., phase angle ((I)) is chosen to be approximately equal to zero).

FIG. 2D depicts oscillatory flow 290 entering catheter tip 230 (in the proximal direction); thrombus 240 is shown translated proximally and attachment 260 is shown stretched in the proximal direction. Oscillatory flow 290 is shown with a longer arrow in FIG. 2D than in FIG. 2A; this illustrates that a net aspiration inflow exists during this representative cycle.

In FIG. 2A through FIG. 2D, thrombus 240 is shown to oscillate through amplitude 300 by comparing relative thrombus 240 positions in FIG. 2B (distally extended) to FIG. 2D (proximally retracted). Amplitude 300 may be a function of system process variables (e.g., factors, levels, setpoints, adjustments, etc.) including frequency and stroke of LF oscillator 102 and/or harmonic oscillator (HO) 104. In some embodiments, amplitude 300 is at or near maximum when the frequency is at or near a natural frequency of the system comprising thrombus 240, attachments 260 and vessel wall 280. LF oscillator 102 is capable of generating flow at a variety of different frequencies, more than one of which may cause thrombus 240 to rock, twist, or rotate with respect to vessel wall 280. Other frequencies of flow generated by the system (including harmonic oscillator (HO) 104) are likely to cause internal vibrations within thrombus 240, and compromise the structural integrity thereof.

In FIG. 2E through FIG. 2G, thrombus 240 is depicted in a substantially stationary position, while vessel wall 280 is depicted as being excited (in vibrational modes) at three representative frequencies, which may be resonant frequencies. FIG. 2E through FIG. 2G depict instantaneous “snapshots” of vessel wall 280 at any given instant. Each of FIG. 2E through FIG. 2G depict different configurations (shapes) of vessel wall 280, as results from the vibrational mode excited at any given instant in time. It is possible that standing waves will develop in the vasculature or vessel wall.

In FIG. 2E, vessel wall 280 is shown to be instantaneously distended; this is representative of a first vibrational mode. In this mode, thrombus 240 remains stationary while attachments 260 are stretched (during the distention phase of the oscillation). An associated contraction phase is not shown, wherein vessel wall 280 will have a concave form.

FIG. 2F depicts vessel wall 280 assuming a different shape (from FIG. 2E) wherein an undulating profile is observed; this is representative of a second vibrational mode. In this mode, thrombus 240 remains substantially stationary while some attachments 260 are stretched and other attachments 260 are compressed. FIG. 2G depicts vessel wall 280 assuming a different undulating profile (from FIG. 2F); this is representative of a third vibrational mode.

The three vibrational modes depicted in FIG. 2E through FIG. 2G are but a few of the many vibrational modes that vessel wall 280 may experience as a function of the operation of LCO 100. Similarly, the vibrational modes depicted in FIG. 2A through FIG. 2D are representative of innumerable vibrational modes that thrombus 240 may experience. In some embodiments, LF oscillator 102 sweeps or continuously varies frequency throughout a range, and may therefore excite vibrational modes of either or both thrombus 240 and vessel wall 280. The vibrational modes of thrombus 240 and vessel wall 280 may be coupled to one another, which may cause constructive or destructive interference of the two vibrating structures.

The vibrational oscillatory motion of thrombus 240 and vessel wall 280 as depicted in FIG. 2A through FIG. 2G depicts extension and contraction of attachment 260 by changing the distance between thrombus 240 and vessel wall 280. This relative motion repetitively stresses attachment 260, effectively breaking the physical connection such that thrombus 240 is dislodged for aspiration. LF oscillator 102 imparts relative motion between thrombus 240 and vessel wall 280; this motion may be oscillatory and may be a form of generalized harmonic motion. The LCO 100, LF oscillator 102 and/or harmonic oscillator HO 104 thrombectomy system may also impart internal, vibrational stresses within thrombus 240, which leads to partial, significant, or total disintegration of thrombus 240 independently of the relative motions between thrombus 240, vessel wall 280 and attachment 260.

Some embodiments of the invention analyze data predicated upon deterministic events of the thrombectomy procedure underway and assess thrombectomy efficacy; this may be expanded to include measurement of blood pressure at or near catheter tip 230. An intra-procedural change in blood pressure may also be a quantitative indicator of overall thrombectomy efficacy. For instance, in procedures wherein a catheter is positioned downstream of a thrombotic lesion (i.e., downstream approach), increases in measured blood pressure at or near catheter tip 230 may be indicative of increased flow past a thrombotic lesion. Likewise, in procedures wherein a catheter is positioned upstream of a thrombus (i.e., upstream approach), decreases in measured blood pressure at or near catheter tip 230 may be indicative of increased flow past a thrombotic lesion. Measured increases in native blood flow rate may rationally be attributed to a summation of deterministic events preceding the measurements and also to the current state of thrombectomy efficacy. Some embodiments of the invention thereby provide intra-procedural diagnostic information quantifying the overall (present-state) efficacy of the procedure at any time during the procedure. Thrombus 240 is generally depicted as a solid mass which restricts blood flow through the vasculature; therefore a differential pressure is typically existent on opposite sides of thrombus 240.

During the course of a thrombectomy procedure, as thrombus 240 is disintegrated, ablated or otherwise diminished in size, native blood flow rate will increase; the consequent (quantitatively measured) change in differential pressure is utilized in some embodiments to intra-procedurally quantify the improvement in native blood flow rate. In procedures wherein catheter 220 is deployed on the downstream side of occlusive thrombus 240 (herein referred to as “downstream approach”), local blood pressure at catheter tip 230 is typically diminished below optimal values. In procedures wherein catheter 220 is deployed on the upstream side of occlusive thrombus 240 (herein referred to as “upstream approach”), local blood pressure at catheter tip 230 is typically augmented above optimal values.

FIG. 2G is illustrative of both upstream approach and downstream approach thrombectomy procedures; a significant difference being the direction of native blood flow. Aspirate flow 295 is depicted in the right-to-left orientation; native blood flow direction may be either parallel or anti-parallel to the direction of aspirate flow 295, depending upon the approach. Downstream approach procedures are performed by deploying catheter tip 230 downstream of thrombus 240. In downstream approaches, native blood flows in aspiration flow direction 294, thereby locating catheter tip 230 on the low pressure side of thrombus 240. In downstream approaches, pressure p1 298 is typically less than pressure p2 299, because thrombus 240 presents a flow restriction. Conversely, upstream approach procedures are performed by deploying catheter tip 230 upstream of thrombus 240. In upstream approaches native blood flows in infusion flow direction 296 (which is anti-parallel to aspiration flow direction 295), thereby locating catheter tip 230 on the high pressure side of thrombus 240. In upstream approach procedures, pressure p1 298 is typically greater than pressure p2 299, because pressure p1 298 is on the upstream side of flow restriction thrombus 240. Intra-procedurally, pressure p1 298 (and time dependent fluctuations, e.g., cardiac cycle) may be measured at any time, whereas pressure p2 298 may generally not be measured without complications such as “crossing the lesion.” Real-time measurements of blood pressure at or near catheter tip 230 may be taken at any time during a thrombectomy procedure by reducing the aspiration rate (in this context, absolute flow rate) through the catheter to zero or near zero by means such as stopping aspirate pump 440 or closing a valve in alternative embodiments (not shown). At zero or near zero absolute flow rate, pressure p1 298 may be approximately measured by pressure transducer 420 in fluid communication with catheter 220. The accuracy of the blood pressure measurement at or near catheter tip 230 may be improved by substituting saline (μ≈1 cP) for blood (μ≈4 cP) by means such as saline flush or saline exchange because any non-zero flow rate is accompanied by a pressure drop.

Embodiments of the invention which measure pressure p1 298 proximate to thrombus 240 provide diagnostic information regarding any change in native blood flow rate around thrombus 240. Thrombus 240 is assumed to be quantitatively occlusive (e.g., 20%, 40%, 60%, 100%, etc.); under flowing conditions, a differential pressure between pressure p1 298 and pressure p2 299 will therefore typically exist. In an example downstream approach DVT (Deep Vein Thrombosis) thrombectomy procedure, pressure p1 298 may be initially measured to be approximately 20±3 mmHg. Midway through the procedure, pressure p1 298 may be measured to be approximately 25±4 mmHg and, at the conclusion of the procedure pressure p1 298 may be measured to be approximately 30±5 mmHg. The example 50% increase in average/nominal pressure p1 298, as a result of the thrombectomy procedure, quantitatively measures improvement in native blood flow. In cases of upstream approach thrombectomy procedures, measured pressure p1 298 may be expected to exhibit a decrease as result of the procedure. The initial, intermediate, and final measurement data (of pressure p1 298) may be correlated to predetermined values (e.g., historical data, database, knowledge base, etc.), such that overall procedure efficacy may be quantitatively measured in terms of changes in blood pressure at or near the site of the lesion (thrombus). Thusly, some embodiments of the invention measure native blood flow rate—external to the catheter—by measuring flowing pressure proximate to a thrombus. This is in contrast to prior art methods which collect pressure data and determine “characteristics of flow” internal to the catheter. Prior art is limited to monitoring (or perhaps measuring) blood flow characteristics or states within the catheter; in contrast, some embodiments of the invention measure intravascular blood flow.

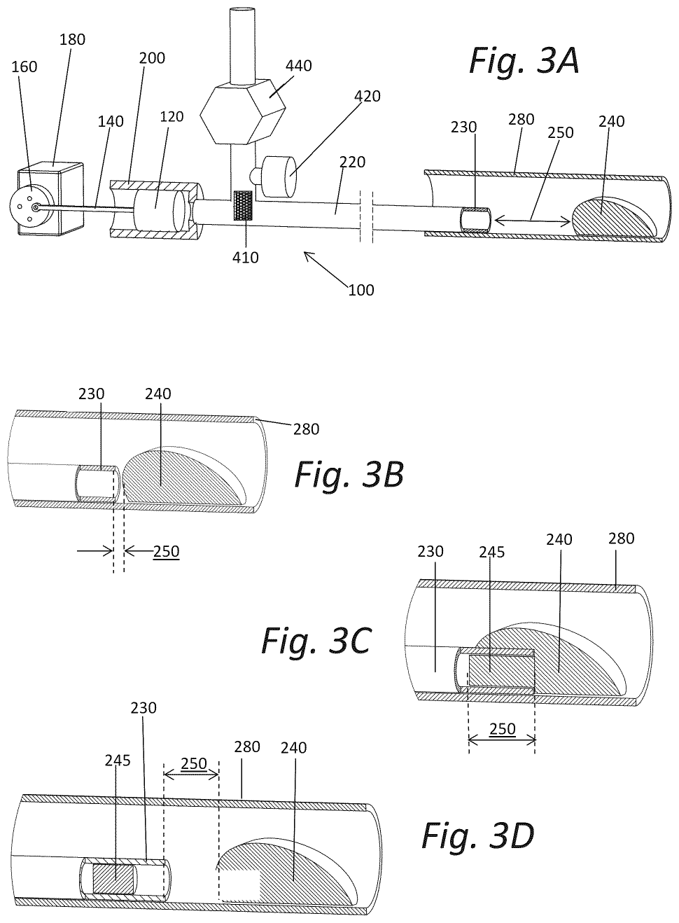

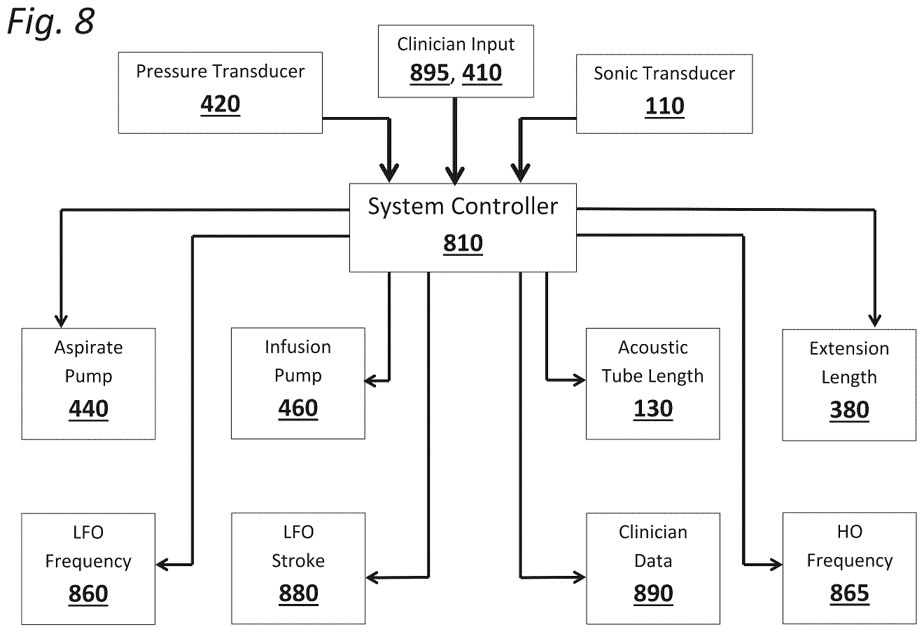

FIG. 3A through FIG. 3D illustrate an embodiment and example methodology to viscometrically detect the distance between catheter tip 230 and thrombus 240 (herein, viscometric distance sampling); this distance is shown in FIG. 3A through FIG. 3D as tip to thrombus distance 250. In this example embodiment, system controller 810 (depicted in FIG. 8 and FIG. 15) receives data from system instrumentation including pressure transducer 420 and accelerometer 410, and exerts control over system components including aspirate pump 440 and motor 180. Some embodiments of the present invention utilize a method that takes viscometric measurements upon a small sample volume (range of 0.2cc to 5cc), typically in less than one second (range of 0.2s to 5s). If this small sample of aspirate is measured to have viscosity consistent with that of blood (≈4 cP), the sample may be determined to be “pure” or “unadulterated with thrombus;” this pure sample may be returned to the bloodstream by means such as reversal of aspirate pump 440. Thusly, aspirate samples may be viscometrically measured with negligible, zero or near-zero blood loss.

FIG. 3A depicts catheter tip to thrombus distance 250 as “too large” for efficacious thrombus aspiration; catheter tip to thrombus distance 250 is shown to be greater than the diameter of vasculature generally bounded by vessel wall 280. Application of vacuum or suction in this configuration typically results in blood loss without successful harvesting of thrombus 240. The measured viscosity of aspirate samples is typically approximately equal to that of blood. System controller 810 (depicted in FIG. 8 and FIG. 15) may indicate to the clinician that the catheter tip to thrombus distance 250 is too large by any communication means including audiovisual. The clinician may be thereby advised to advance or otherwise reposition the catheter 220, this occurring with a minimum of blood loss in making the distance determination. In alternative embodiments the physical act of advancing, retracting or repositioning of catheter 220 into proper catheter tip to thrombus distance 250 is mechanically executed under mechanized or automated control.

FIG. 3B depicts catheter in an example optimal position; catheter tip to thrombus distance 250 is shown to be less than the diameter of catheter tip 230. Application of vacuum or suction in the configuration of FIG. 3B may cause thrombus 240, or a portion thereof, to enter catheter tip 230. The measured aspirate viscosity for the configuration of FIG. 3B may range from approximately 4.1 cP to in excess of approximately 50 cP, with a greater measured viscosity being indicative of a smaller catheter tip to thrombus distance 250. As catheter 220 is continuously or incrementally advanced toward thrombus 240, system controller 810 measures a continuous or incremental increase in aspirate viscosity; this viscosity measurement being indicative of the magnitude (and sign) of catheter tip to thrombus distance 250. An optimum catheter tip to thrombus distance 250, (or range of distances) as measured viscometrically may be experimentally determined; this predetermined value may be stored in memory, firmware or software of system controller 810.

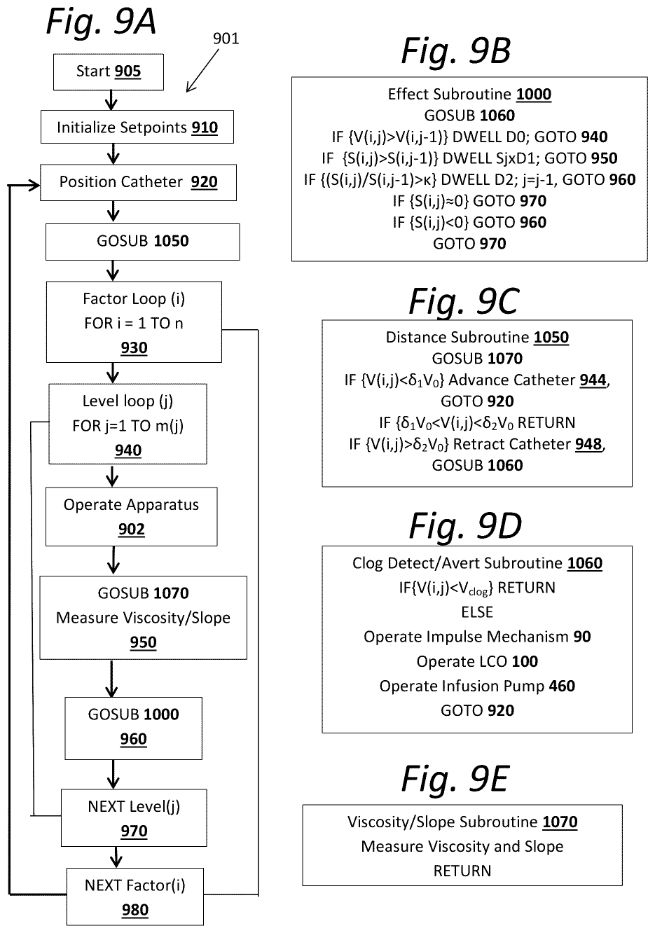

FIG. 3C depicts a negative catheter tip to thrombus distance 250 wherein catheter tip 230 has pierced or penetrated thrombus 240, forming thrombus bolus 245. The measured aspirate viscosity for the configuration of FIG. 3C may range from approximately 51 cP to in excess of 10,000 cP. The configuration of FIG. 3C depicts a scenario wherein a clog or imminent clog is viscometrically detected by system controller 810. System controller 810 may respond to the measured catheter tip to thrombus distance 250 in one or more ways including: mechanically or advising the clinician to advance or retract catheter 220, applying vacuum/suction levels (e.g., reduced, intermittent, interspersed, reversing, etc.) appropriate to the clinically measured catheter tip to thrombus distance 250. System controller 810 employs thrombectomy control flowchart 901, algorithm and/or subroutine (such as “distance” subroutine 1050, subsequently presented in conjunction with FIG. 9C) to invoke a prescribed course of action (e.g., repositioning of catheter 220, speed of aspirate pump 440, speed of infusion pump 460, etc.). Prescribed course of action may be determined by system controller 810 by means including algebraic, ratiometric or other quantitative method that simultaneously minimizes blood loss and likelihood of clogging or corking catheter 220.

FIG. 3A through FIG. 3C depict embodiments and methods to efficaciously aspirate thrombus 240 by incorporating measurements of system parameters including viscosity, pressure, % thrombus and/or relative flow rate; these measurements are detailed in co-pending applications listed as references. FIG. 3A depicts a partial cutaway view of LF oscillator 102 in an embodiment that includes: aspirate pump 440, pressure transducer 420 and accelerometer 410. Motor 180 rotates crankshaft 160 such that connecting rod 140 and piston 120 are rotated and/or translated; piston 120 is shown at or near TDC (Top Dead Center) of cylinder 200, shown in cutaway view. Rotation of crankshaft 160 (up to approximately 180° from TDC) causes piston 120 to move leftward, thereby reducing pressure within catheter 220. Sufficiently rapid rotation of crankshaft 160 (up to approximately 180° from TDC) may effect cavitation or boiling pressure to develop within cylinder 200 and catheter 220; alternate embodiments include syringes, linear actuators, evacuated reservoirs, etc. that are equivalently employed to generate low absolute pressure conditions including cavitation or boiling pressure. Cavitation or boiling pressure (within cylinder 200 and/or catheter 220) may represent the minimum pressure physically attainable and may therefore generate the maximum attainable suction to aspirate thrombus 240. Thus, the system in FIG. 3A is shown poised or primed to activate, engage, supply or deliver maximum aspiration suction/vacuum upon rapid rotation of crankshaft 160 or other embodiment analogue such as a syringe. In FIG. 3A however, catheter tip to thrombus distance 250 is measured to be “too large” for activation or engagement of significant aspiration suction/vacuum.

The embodiment and configuration of FIG. 3A illustrates another feature of the present invention: the rapid aspiration of a limited volume of aspirate by rapidly rotating crankshaft 160 through angular displacement of approximately 180° (or less). An angular rotation of approximately 180° may occur in less than 1 second (range of approximately 0.1s to 3s). Using the dimensions of a previous example, LF oscillator 102 of dimensions of 1 cm bore and 1 cm stroke and exhibits a displacement of approximately 0.8cc. Thusly, the depicted embodiment and configuration of FIG. 3A is capable of increasing the system volume by approximately 0.8cc in less than one second. In some clinical situations (catheter dimensions, % thrombus, etc.) this volumetric displacement is rapid enough to develop cavitation or boiling pressure within cylinder 200. This embodiment and method provide two desirable attributes: (1) minimum pressure is developed for maximum initial aspiration rate, and (2) the volume of aspirate is limited to the displacement of piston 120 (in this example case, 0.8cc). This is in contrast to prior art thrombectomy systems wherein an evacuated reservoir, syringe or peristaltic pump imposes no limitation on the volume aspirated. Furthermore, the detailed embodiment and method may reduce the likelihood of clogging or corking a catheter 220 due to over-ingestion of thrombus 240. This method enables embodiments of the invention to aspirate thrombus 240 as discretized thrombus bolus 245, which may be sequentially aspirated, thereby avoiding over-ingestion, clogging and/or corking.

In the configuration of FIG. 3C, depicting “negative” catheter tip to thrombus distance 250, catheter 220 may be withdrawn slightly (range of approximately 1 to 5 times the diameter of catheter 220) such that successive aspiration is comprised predominantly of blood such that discrete thrombus bolus 245 is successfully aspirated without incurring measured viscosity levels that risk clogging or corking. Embodiments of the present invention thereby limit the measured level of % thrombus by judiciously by withdrawing and/or advising the clinician to withdraw catheter 220 such that blood “dilutes” the aspirate composition to exhibit a flowable characteristic under aspiration suction/vacuum.

FIG. 3D depicts a configuration wherein catheter tip 230 has been repositioned (i.e., withdrawn) proximally from the configuration depicted in FIG. 3C; catheter tip to thrombus distance 250 is shown positive such that blood may flow into catheter tip 230 under aspiration. This repositioning of catheter 220 may be effected by automated means or by advising a clinician to execute the withdrawal or retraction. Thrombus bolus 245 is depicted to be separated from thrombus 240, and may be aspirated discretely from thrombus 240. In this manner, the bulk or whole of thrombus 240 may be aspirated as a plurality of discrete instances of thrombus bolus 245. In the configuration of FIG. 3D, thrombus bolus 245 may be aspirated at maximum or near-maximum vacuum/suction provided that viscometric sampling returns a measurement less than a value predetermined to be indicative of a clogged or corked catheter.

Objectives of some embodiments of the present invention include the measurement, determination or detection of the catheter tip to thrombus distance 250. A knowledge-based thrombectomy system may subsequently regulate, adjust or modulate the vacuum/suction (including cavitation or boiling pressure) to levels appropriate for system parameters including the catheter tip to thrombus distance 250. A further objective of the present invention is to regulate, adjust or modulate the vacuum/suction to levels such that aspiration of thrombus 240 occurs with a minimized occurrence of clogging or corking of catheter 220 or catheter tip 230; clogging or corking of catheter 220 or catheter tip 230 may be exacerbated if maximum or near-maximum levels of suction/vacuum are developed.

FIG. 3B depicts catheter tip 230 and thrombus 240 to be separated from one another by distance smaller than in FIG. 3A and is purported to be representative of a first favorable or desired catheter tip to thrombus distance 250; values may range from 0 mm to less than approximately the blood vessel diameter or between 0 mm to less than approximately the three times catheter diameter. Catheter tip 230 is depicted in close proximity to thrombus 240 such that bloodflow into catheter tip 230 may be partially impeded or occluded by thrombus 240. Delivering maximum or near-maximum vacuum/suction levels (in the configuration of FIG. 3B) may result in efficacious aspiration of thrombus 240. Accelerometer 410 is shown affixed to catheter 220 such that the clinician input of advancing catheter tip 230 into proximity to thrombus 240 may be measured and the data stored and/or correlated to changes in aspirate viscosity. Accelerometer 410 may be a 3-axis or 6-axis (including “gyroscopic”) accelerometer or other absolute or relative position sensor. As an example, catheter 220 may have been advanced 4 mm and rotated 90° clockwise to assume the configuration of FIG. 3B. Measured viscosity may have increased from approximately 4 cP (in FIG. 3A) to approximately 10 cP (in FIG. 3B) because inflow of blood into catheter tip 230 may be impeded by the close proximity to thrombus 240 (approximate range of 0.01 mm to 3 mm) without actually penetrating thrombus 240. Appropriate values for measured viscosity (e.g., 10 cP, 30 cP, 50 cP, etc.) which correspond to a favorable or desired catheter tip to thrombus distance 250 may be determined by simple experimentation.

FIG. 3C depicts a configuration wherein catheter tip 230 has penetrated thrombus 240 and a thrombus bolus 245 has formed; data from accelerometer 410 may quantify the linear and rotational speeds and distances imparted to catheter 220 during this manipulation or maneuver. Catheter tip to thrombus distance 250 is shown to be negative in FIG. 3C; this configuration may arise in the absence of any suction/vacuum being applied because the catheter tip 230 may physically pierce soft variants of thrombus 240. Delivering maximum vacuum/suction levels (in the configuration of FIG. 3C) may result in clogging or corking of catheter tip 230 such that ancillary procedures (e.g., device removal, manual manipulation of guidewire, macerator or obturator, etc.) may be required to clear the clog. The severity of such a clog may be directly related to the vacuum/suction level applied. Some embodiments of the present invention regulate or modulate the vacuum/suction level such that thrombus bolus 245 may be aspirated in a controlled manner such that clogging or corking is avoided. Viscosity measurements for the configuration of FIG. 3C may exceed 500 cP, indicating a clog or impending clog. In some embodiments, data from accelerometer 410 (e.g., advance catheter 220 2 mm at 1 mm/second, rotate counterclockwise 90°, etc.) is correlated to the measured viscosity to determine the efficacy of the manipulation or maneuver.

Some embodiments of the present invention include the determination of an appropriate vacuum/suction level for efficacious aspiration of thrombus 240 or thrombus bolus 245 while minimizing blood loss and phenomena including clogging or corking which may require manual clinician intervention. FIG. 3A is representative of a catheter tip to thrombus distance 250 which may be determined or inferred to be too large for effective aspiration of thrombus. In this configuration, thrombectomy operating modes such as viscometric sampling may be indicated as effective treatment as catheter tip 230 is advanced into closer proximity to thrombus 240. FIG. 3B depicts a representation of a catheter tip to thrombus distance 250 which may be determined or inferred to be appropriate for maximum vacuum/suction levels; limiting the duration of the maximum vacuum/suction levels may be important to facilitate aspiration of thrombus bolus 245 and to avoid the phenomenon of clogging or corking. FIG. 3C is representative of a (negative) catheter tip to thrombus distance 250 which may be determined to be appropriate for intermediate vacuum/suction levels including of short duration; an objective may be to isolate thrombus bolus 245 in order that it may be aspirated discretely from thrombus 245. This may be accompanied or facilitated by withdrawing catheter 220 proximally in order that thrombus bolus 245 becomes physically separated from thrombus 240 for more efficacious aspiration by limiting the % thrombus concentration (or viscosity or relative flow rate) of fluid within catheter 220.

FIG. 3A through FIG. 3D depict spatial differences which are represented by catheter tip to thrombus distance 250, illustrated as positive and excessive (in FIG. 3A), positive and within optimal range (in FIG. 3B), negative (in FIG. 3C) and positive and within optimal range (in FIG. 3D). This spatial difference may be detected by the measured viscosity, % thrombus or relative flow rate through catheter 220. In FIG. 3A, fluid flow into catheter tip 230 is shown to be unoccluded and the relative flow rate may be measured as maximum by flow measurement means including time-domain viscometry. In FIG. 3B, fluid flow into catheter tip 230 is shown to be partially obscured and a diminished relative flow rate may be measured as an increase in viscosity. In FIG. 3C, fluid flow into catheter tip 230 is shown to be effectively occluded and a relative flow rate that is very low or approaching zero may be measured at low vacuum/suction levels. In this configuration, application of high vacuum/suction levels may induce clogging or corking of catheter 220

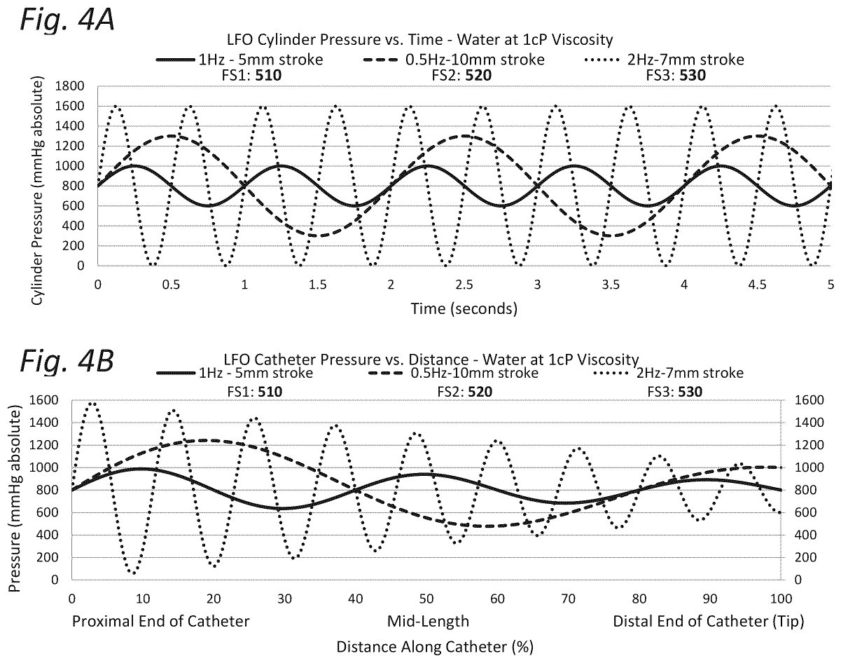

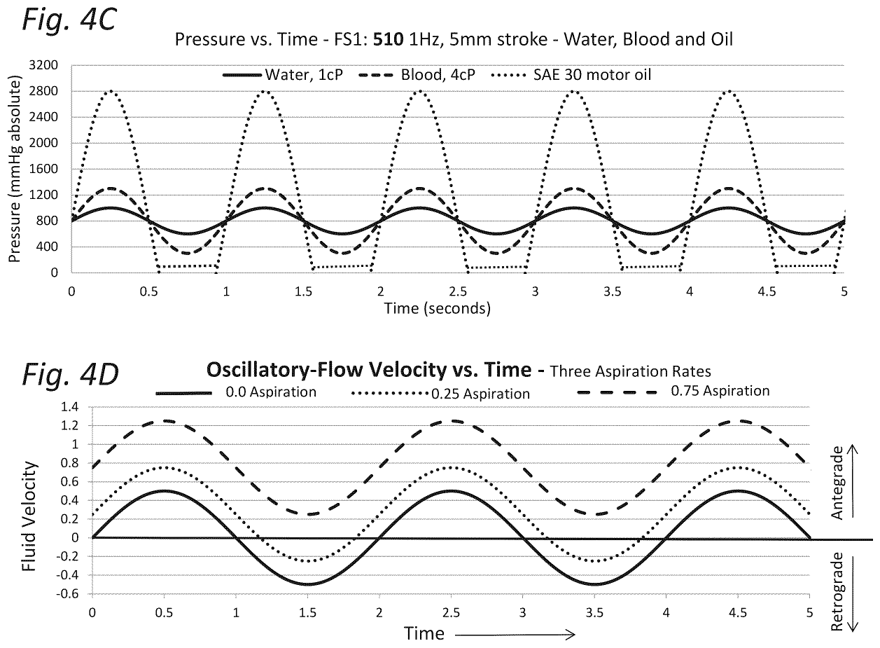

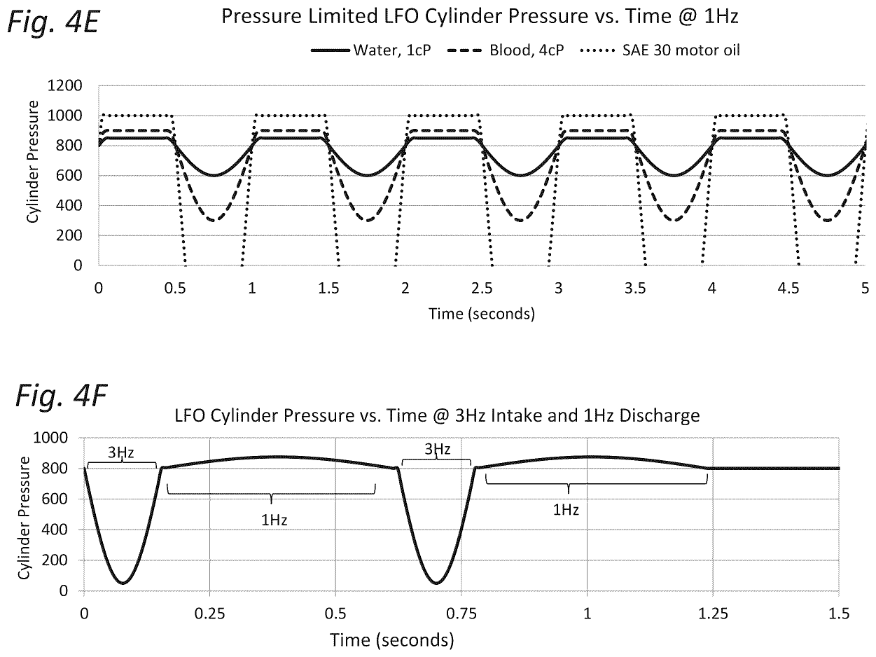

Some embodiments of the present invention may include methods wherein thrombus bolus 245 (as shown in FIG. 3C and FIG. 3D) may be aspirated in conjunction with proximal withdrawal (range between approximately 0.5 mm and 10 mm) of catheter 220 (i.e., to the example configurations of FIG. 3A and/or FIG. 3B). The configuration of FIG. 3D depicts catheter tip to thrombus 250 to be positive and sufficient that blood may flow into catheter tip 230 thereby isolating thrombus bolus 245. Thereby, each discrete thrombus bolus 245 may traverse catheter 220 proximally by means including vacuum/suction aspiration and/or oscillatory fluid motion. Thus, thrombus bolus 245 may be aspirated extracorporeally, while the remaining portion of thrombus 240 remains effectively intact and awaiting aspiration by successive processes. FIG. 3A through FIG. 3D illustrate incorporation or integration of relative flow rate measurement (by means including viscometric sampling and/or time-domain viscometry); this leads to a quantitative determination of appropriate vacuum/suction levels and durations to efficaciously aspirate thrombus independently of any ancillary features or system components (e.g, LCO 100, LF oscillator 102 and or harmonic oscillator HO 104, hydrodynamic lance, macerator, obturator, etc.). Catheter 220 is shown in 4 different axial positions in FIG. 3A, FIG. 3B, FIG. 3C and FIG. 4D (rotation may or may not have occurred) by means including clinician input (manual manipulation) and/or automated or mechanized catheter positioning. Concomitantly, accelerometer 410 may measure, store and/or correlate the displacements to measured thrombectomy efficacy. In some embodiments catheter tip to thrombus distance 250 is effected mechanically enacted by means of system controller 810 and motors, actuators, grippers, etc. (not shown).

Example control responses from system controller 810 are further detailed in conjunction with FIG. 9A through FIG. 9D. Example thrombectomy system control responses for each configuration of FIG. 3A through FIG. 3D may be defined such as:
a. FIG. 3A, catheter tip to thrombus distance 250 is positive and excessive. Unimpeded flow is measured (e.g., relative flow rate≈100% or μ≈4 cP or). Example control responses include: viscometric sampling, viscometric distance sampling, approximately 5% to 20% aspiration rate.
b. FIG. 3B, catheter tip to thrombus distance 250 is positive and within efficacious range. An intermediate flow restriction or obstruction is measured (e.g., relative flow rate is between approximately 20% and 90% or 10 cP<μ<200 cP). Example control responses include: approximately 50% to 100% aspiration rate, invoke other system parameters, factors or means to attrite thrombus (e.g., LF oscillator 102, HO 104, infusion, etc.).
c. FIG. 3C, catheter tip to thrombus distance 250 is negative, clogging may occur. A large flow restriction or obstruction is measured (e.g., relative flow rate is between approximately 0% and 10% or μ>400 cP). Example control responses include: volume-limited aspiration, impulse mechanism 90, repositioning of catheter 220, clog detect/avert subroutine 1060, etc.).
d. FIG. 3D, catheter tip to thrombus distance 250 is positive and within efficacious range, thrombus bolus 245 is within catheter tip 230. Relative flow rate may be increased from FIG. 3C (e.g., relative flow rate may be between approximately 0% and 100% or 4 cP<μ<10,000 cP); example control responses include: volume-limited aspiration, impulse mechanism 90, maximum aspiration, viscometric sampling, LF oscillator 102, harmonic oscillator 104, etc.). Some example control responses are codified in example subroutines, (e.g., distance subroutine 1050 and example clog detect/avert subroutine 1060 or similar), as depicted in FIG. 9C and FIG. 9D.

FIG. 4A depicts fluid pressure vs time waveforms calculated (using Eq. 1 through Eq. 8) to exist within cylinder 200 of low frequency oscillator 102; three example combinations of frequency and stroke are depicted. In FIG. 4A the fluid is water or saline with viscosity of approximately 1 cP. Three characteristic waveforms exist; one waveform for each combination of frequency and stroke. A combination of frequency and stroke may be herein termed FSn, where n represents the n^{th} such combination under consideration; an example is FS1 (abbreviation for Frequency/Stroke combination 1). FIG. 4A depicts three Frequency/Stroke combinations: FS1 510 (1 Hz, 5 mm stroke), FS2 520 (0.5 Hz, 10 mm stroke) and FS3 530 (2 Hz, 7 mm stroke). With water or saline as working fluid, the amplitude of each waveform (FS1 510, FS2 520 and FS3 530) is generally a function of parameters including stroke, catheter 220 length, diameter and elasticity; the period is generally a function of frequency. The ordinate of FIG. 4A has units of mmHg absolute pressure. Herein it is assumed that the average intravascular pressure may be approximately 40 mmHg (above atmospheric pressure, 760 mmHg) and that the average absolute pressure may be approximately 800 mmHg; 800 mmHg absolute may be termed nominal, intravascular or ambient pressure herein. In accordance with Design Of Experiments (DOE) nomenclature the frequency and stroke (e.g., of low frequency oscillator 102) may be considered factors; specific or approximate frequencies (e.g., 1 Hz, 20 Hz, 37 Hz, etc.) and strokes (e.g., 1 mm, 3.65 mm, 5 mm, etc.) may be considered levels. The example frequency/stroke combinations (FSn) illustrate a two factor (frequency and stroke), three level (0.5 Hz, 1 Hz, 2 Hz and 5 mm, 7 mm and 10 mm) experiment design.

FIG. 4A illustrates that an LF oscillator 102 (operating at approximately constant angular velocity) theoretically generates approximately sinusoidal pressure waveforms. Waveforms depicted in FIG. 4A (FS1 510, FS2 520 and FS3 530) may be representative of a particular LCO 100 or LF oscillator 102 embodiment; other system component variables including catheter 220 length, diameter and elasticity also influence the amplitude. A short, large diameter catheter 220 may generally operate at lower pressure amplitudes because: (1) for a given differential pressure, a shorter column of liquid may accelerate faster than a longer column of the same liquid because of the difference in mass, and (2) a larger diameter catheter 220 enables greater mass flow at lower velocities with correspondingly less viscous dissipation (frictional losses). The viscosity of the fluid contained within catheter 220 also affects the waveform amplitude. A more viscous fluid requires a greater differential pressure for any given fluid oscillation amplitude, therefore a more viscous fluid typically exhibits waveforms of greater pressure amplitude.

In FIG. 4A, pressure waveform FS1 510 exhibits amplitude of approximately ±200 mmHg superposed over an 800 mmHg nominal pressure; waveform FS2 520 exhibits amplitude of approximately ±500 mmHg and waveform FS3 530 exhibits amplitude of approximately ±800 mmHg. Waveform FS3 530 correspondingly depicts a minimum pressure of 0 mmHg absolute; cavitation or boiling may occur at any pressure at or below approximately 50 mmHg (the approximate vapor pressure of blood/water at 37° C.). Certain embodiments of an LCO or knowledge based thrombectomy system may employ cavitation within cylinder 200 for reasons including: (1) the physical limits of low pressure (e.g., approximately 50 mmHg absolute) are generated for maximum dislodgement and aspiration effects and (2) the collapse of cavitation or boiling bubbles or voids in cylinder 200 may generate shock waves that traverse the length of catheter 220 and directly impinge upon thrombus 240 and/or attachment 260. Some methods, embodiments and process variables (e.g., FS3, FS5, FS7, FS9, etc.) of the present invention include cavitation-inducing waveforms which generate alternating sequence of maximum vacuum and shock-induced pressure waves; whereas other process variables (e.g., FS1, FS2, FS4, etc.) deliver alternate or non-cavitating waveforms including: other therapeutic waveforms, diagnostic waveforms or viscometric waveforms.

FIG. 4B depicts a corresponding pressure (amplitude) decay which typically occurs along the length of catheter 220; the pressure decay observed in FIG. 4B may be attributed to factors including: (1) viscous friction (resistance) and (2) system compliance (capacitance). FIG. 4B illustrates that even though pressures exceeding 2bar (1,500+mmHg) absolute and full vacuum may occur within cylinder 200; the amplitude of each pressure waveform is diminished with distance along catheter 220 away from cylinder 200. For the example combination of LCO 100 and FS1 510, FS2 520 and FS3 530 (with water as the working fluid, i.e., the fluid contained within catheter 220) it is evident that the pressure amplitude present at catheter tip 230 is diminished from the pressure amplitude generated within cylinder 200. FS1 510 exhibits pressure amplitude of approximately 200 mmHg within cylinder 200; the pressure amplitude at catheter tip 230 is diminished to approximately 100 mmHg. FS2 520 exhibits pressure amplitude of approximately 500 mmHg within cylinder 200; the pressure amplitude at catheter tip 230 is diminished to approximately 200 mmHg. FS3 530 exhibits pressure amplitude of approximately 800 mmHg within cylinder 200; the pressure amplitude at catheter tip 230 is diminished to approximately 200 mmHg. FIG. 4B is included to illustrate that any pressure or vacuum condition that exists (including extreme examples such as cavitation or boiling) at or near the proximal end of a catheter may not be inferred to be present at the distal end of the catheter, particularly under flowing conditions. FIG. 4B generally illustrates the dissipative (i.e., frictional) effect of viscosity in a fluid. In cases where aspirate is flowing through a catheter, the greater the fluid velocity, the greater the dissipative losses incurred along the length of the catheter. In cases wherein the flow velocity is at or near zero, dissipative losses are at or near zero. In cases of a clogged catheter, a pressure discontinuity may exist across the clog; however, with near zero flow, there exists nearly zero dissipative (frictional) losses as result of viscosity. FIG. 4B is included to illustrate that measuring the pressure at the proximal end of catheter 220, in conjunction with a measurement of aspirate viscosity or flow rate, enables an inference of the pressure (and/or pressure amplitude) at catheter tip 230.

Some embodiments of the present invention generate a plurality of continuous or discrete pressure waveforms (e.g., FS1 510, FS2 520, FS3 530, . . . , FSn) such that a spectrum of waveforms may be delivered to the target site, e.g., thrombus 240 and surrounding tissue. This increases the likelihood of generating one or more waveforms which excite one or more vibrational modes of any or all of: (1) thrombus 420, (2) attachment 460, (3) vessel wall 280, and (4) catheter 220.

FIG. 4C depicts a representative functional relationship between pressure waveforms and fluid viscosity; FS1 510 is the frequency/stroke setting of LF oscillator 102; the fluids of different viscosity are shown to generate pressure amplitude waveforms that are typically proportional to the fluid viscosity. In FIG. 4C, water (≈1 cP viscosity) is shown to generate the same FS1 510 waveform/pressure amplitude as shown in FIG. 4A; water's pressure amplitude is shown to be approximately ±200 mmHg. Blood (≈4 cP viscosity) is shown to generate a waveform with pressure amplitude greater than that of water; blood's pressure amplitude is shown to be approximately ±500 mmHg. SAE 30 motor oil (≈35 cP viscosity) is shown to generate a waveform with pressure amplitude of approximately +2,000/−800 mmHg. The pressure amplitude for SAE 30 motor oil indicates that cavitation or boiling may occur because the waveform shows pressures less than approximately 50 mmHg absolute (this being dependent upon parameters including the vapor pressure of SAE 30 motor oil). In cases wherein the pressure falls below the cavitation or boiling pressure (approximately 50 mmHg for saline and/or blood), the positive value of the pressure amplitude is the relevant quantity, because the absolute pressure cannot attain negative values, as shown in FIG. 4C. In some embodiments, an LCO 100 or LF oscillator 102 comprising a pressure measurement device may act as a viscometer; the viscosity of the fluid may be proportional to the (positive) pressure amplitude. In other embodiments an LCO 100 or LF oscillator 102 may act as a viscometer by measuring the current draw to motor 180 because the increased operating pressure (generally within cylinder 200) requires an increase in motor 180 torque.

FIG. 4A, FIG. 4B and FIG. 4C depict representative, ideal pressure vs time waveforms as generated by an LCO 100 or LF oscillator 102; depicted is the differential pressure between the proximal end of catheter and a second pressure, such as intravascular, nominal or atmospheric pressure. These idealized oscillatory pressure waveforms give rise to oscillatory flow within a catheter (assuming homogeneous, inviscid, massless fluid in the absence of cavitation or boiling). This is because depicted embodiments of LCO 100, LF Oscillator 102 and harmonic oscillator HO 104 are positive displacement apparatus that repetitiously increase and decrease the extracorporeal system volume. Idealized oscillatory flow (in the absence of aspiration or infusion) is positive displacement with zero net flow.

Oscillatory flow, as implemented in embodiments of the invention, may be distinguished from other forms of positive-displacement pulsatile flow apparatus such as pumping systems including piston pumps, pulse generators, diaphragm or peristaltic pumps. These example pulsatile pumping systems generally exhibit positive displacement characteristics but are designed to deliver flow in a single flow direction. Oscillatory flow may also be distinguished from flow resultant from pulsed or intermittent application of differential pressure from pressurized or evacuated reservoirs. Pressurized or evacuated reservoirs are typically termed dynamic or kinetic pumping systems and exert no control over (or constraint upon) volumetric (or mass) flow rates. The volumetric (or mass) flow rate of dynamic or kinetic pumping systems is dependent upon other system parameters such as fluid viscosity, catheter diameter/length, restrictions, differential pressure, etc. Systems utilizing pressurized/evacuated reservoirs typically exhibit non-zero net flow; in some cases the flow may be reversing (e.g., by actuation of one or more valves and reservoirs) however mere flow reversal in insufficient to meet the criteria of oscillatory flow.

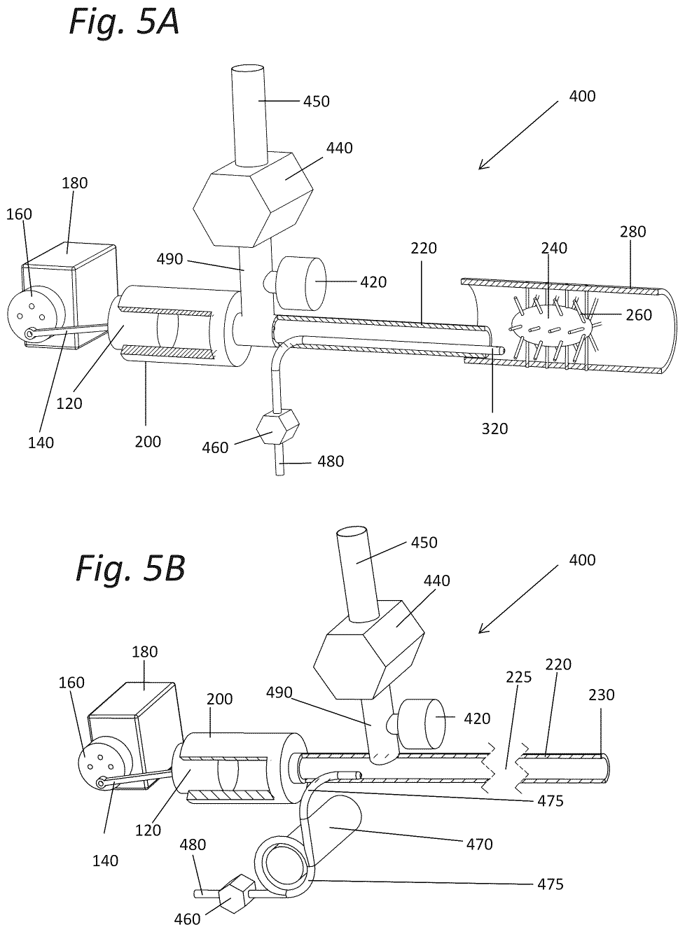

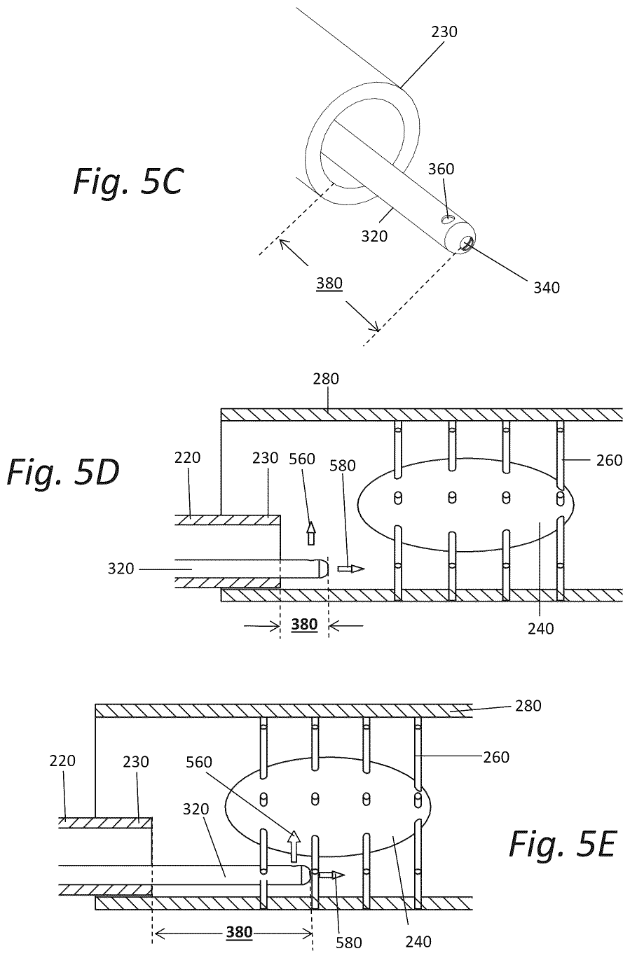

FIG. 4D is presented in conjunction with FIG. 5A wherein net aspirate flow is superposed over oscillatory flow generated by LCO 100 or LF oscillator 102. FIG. 4E and FIG. 4F show a representative, idealized pressure vs time relationship of an LCO 100 or LF oscillator 102 which generates oscillatory flow, however two pressure limiting embodiments are shown: the effect of embodiments such as pressure limiter 10 is shown in FIG. 4E and the effect of variable crankshaft 160 rotational frequency is shown in FIG. 4F. FIG. 4E depicts representative pressure vs time waveforms for three liquids: water (≈1 cP), blood (≈4 cP) and SAE 30 motor oil (≈35 cP) as working fluids in LCO 100 or LF oscillator 102 that includes pressure limiter 10. As in FIG. 4C, the calculated/observed pressure amplitude increases with increasing viscosity, but in FIG. 4E the maximum pressure is limited to a lower pressure to avoid undesirable aspirate outflow from catheter 220. FIG. 4F depicts a representative pressure vs time relationship for a single liquid, however the LCO 100 or LF oscillator 102 is shown to be operated at 3 Hz during the intake stroke and at 1 Hz during the discharge stroke. Allotting more time for fluid to flow outwardly (i.e., distally, or out through catheter 220) during the 1 Hz discharge stroke effectively limits the maximum pressure developed. The resultant net flow remains approximately zero or near-zero, however the flow rate and corresponding cylinder pressure are diminished by the “slow” discharge stroke. Embodiments may comprise the selection of a stepper- or servo-motor as motor 180, which may enable variable angular velocity of crankshaft 160, thereby effecting a “fast” intake stroke and a “slow” discharge stroke to limit the pressure (e.g., above intravascular) developed.

FIG. 5A depicts an embodiment of LCO with aspiration and infusion 400 shown comprised of LF oscillator 102 in conjunction with an aspiration system such as aspirate pump 440 and infusion pump 460. Features such as Harmonic Oscillator (HO) 104 and pressure limiter 10 are omitted in FIG. 5A for clarity. Aspirate pump 440 may intermittently or continuously draw fluid out of the patient and into a reservoir such as a waste reservoir (not shown) or waste tube 450; some embodiments of aspiration systems may provide either net inflow or net outflow from the patient.

Acting alone, aspirate pump 440 may be operated at a steady shaft speed (RPM) and may generate a (steady or pulsatile) unidirectional flow through catheter 220. Acting alone, LF oscillator 102 theoretically generates approximately zero or near-zero net flow (when averaged over time) because the quantity of fluid outflow is typically approximately equal to the quantity of fluid inflow for each cycle. In FIG. 4A, FIG. 4B and FIG. 4C the pressure curves are symmetric about the assumed intravascular pressure of approximately 800 mmHg absolute. FIG. 4A, FIG. 4B and FIG. 4C illustrate oscillatory flow wherein a (generally central to catheter 220) differential fluid volume element within catheter 220 may accelerate, decelerate and reverse repeatedly in the axial direction, returning to approximately the same axial position with each oscillatory cycle.

Acting together, the combined effects of a typical aspiration system and an LF oscillator 102 may be analyzed by the principle of superposition, wherein the net flow of an aspiration system is added to the (ideal) zero-net flow (oscillatory flow) of LF oscillator 102. FIG. 4D depicts the superposition (summation) of the contributions of these two independent systems; the ordinate is fluid velocity within catheter 220. With LCO 100 or LF oscillator 102 operating at FS1 510 (1 Hz, 5 mm stroke, water) and 0.0 aspiration (absolute, relative or arbitrary units), a vertically symmetric velocity curve is shown with maximum and minimum velocity to be approximately ±0.5 (absolute, relative or arbitrary units). This is generally the same curve as in FIG. 4A, however the ordinate is velocity rather than pressure. With aspiration at a flow velocity of 0.25 (absolute, relative or arbitrary units), the velocity curve is shifted upward such that the area above zero is not equal to the area below. The flow is oscillatory in that both positive and negative velocities are shown; there is a net outflow of fluid from the patient. With aspiration at a flow velocity of 0.75 (absolute, relative or arbitrary units), the velocity curve is shifted farther upward such that negative velocities do not exist; there is only outflow from the patient, this is shown as antegrade flow in FIG. 4D. FIG. 4D illustrates that oscillatory flow may exist with equal or unequal velocities in each direction; in the case of aspiration at flow velocity of 0.75, the oscillatory flow is unidirectional (albeit time varying). With the combination of an aspiration system and LCO 100 or LF oscillator 102 operational, the ratio of aspiration velocity to LCO 100 or LF oscillator 102 induced velocity determines whether the resulting flow is bi-directional (balanced or imbalanced) or unidirectional. FIG. 4D depicts three cases of oscillatory flow: balanced bi-directional, imbalanced bi-directional and unidirectional. The fluid velocity scale is shown to be approximately −0.6 to 1.4 (absolute, relative or arbitrary units); these values are selected for graphical representation only. In FIG. 4A, FIG. 4B, FIG. 4C and FIG. 4D, the stated example magnitudes and ranges of pressure and velocity are chosen for graphical and/or illustration purposes only.

FIG. 5A depicts an embodiment of an LCO with aspiration and infusion 400;
additional system components such as harmonic oscillator (HO) 104, acoustic tube 130 and sonic transducer 110 are not shown in FIG. 5A for clarity. Viscometric aspiration (described elsewhere in the references) or viscometric sampling is utilized to determine viscosity and/or the aspirate characteristic (e.g., 6 cP viscosity, Qr=67% relative flow rate, 1cc/sec absolute flow rate, “clot,” “clog,” “free-flow,” “thrombus,” etc.). Saline infusion through infusion tube 320 is incorporated for hydrodynamic effects including: (1) to position or apply force upon catheter 220, (2) to disrupt thrombus 240 and attachment 260 by dilution or direct impingement and/or (3) decrease the viscosity of fluid within catheter 220. The LCO with aspiration and infusion 400 embodiment of FIG. 5A integrates three independent systems to (1) vibrate, (2) mechanically and/or hydrodynamically macerate and (3) aspirate thrombus.

The LCO with aspiration and infusion 400 embodiment depicted in FIG. 5A is comprised of motor 180, crankshaft 160, and connecting rod 140 operating to actuate piston 120 reciprocally within cylinder 200. This combination of components (LF oscillator 102) generates oscillatory flow within catheter 220, as illustrated in previous figures. Manifold 490 fluidically couples catheter 220, pressure transducer 420 and aspirate pump 440; discharge from aspirate pump 440 is to waste tube 450. Aspirate pump 440 may be a positive displacement pump, such as a peristaltic pump, and there may be approximately zero net flow (into or out of the patient) when aspirate pump 440 is stopped. With the LF oscillator 102 operational (i.e., piston 120 is reciprocating) and aspirate pump 440 stopped, a cyclic pressure waveform may be measured by pressure transducer 120; this waveform is characteristic of the fluid generally contained within catheter 220. The (positive, above intravascular) pressure amplitude of the cyclic pressure waveform may be indicative of the viscosity of any fluid generally contained within catheter 220; a larger amplitude pressure waveform may be indicative of a greater fluid viscosity and vice versa. Thus, the LF oscillator 102 and pressure transducer 420 may combine to form a viscometer which measures the viscosity of any fluid generally contained within catheter 220.

Aspirate pump 440 and pressure transducer 420 may also combine to form a viscometer as described elsewhere in the references. Thus, the viscosity of fluid generally contained within catheter 220 may be measured with (1) LF oscillator 102 operating, (2) aspirate pump 440 operating or (3) both LF oscillator 102 and aspirate pump 440 operating simultaneously. Viscometry is utilized in the present invention (and co-pending applications) to quantitatively determine the aspirate viscosity or aspirate characteristic of the fluid contained within catheter 220. If the measured viscosity is low (that of viable blood at approximately 4 cP) then catheter 220 may be inferred be aspirating blood; the speed of aspirate pump 440 may be decreased or maintained at a low speed and/or operated intermittently to minimize the loss of viable blood. If the measured viscosity is greater than viable blood, then catheter 220 may be inferred to be aspirating thrombotic material; consequently, the speed of aspirate pump 440 may be increased or adjusted to maximize the extraction efficiency of thrombotic material. Ratiometric or differential viscometry may quantify any changes in viscosity without reference to an external standard such as a calibration standard. The ratio of measured viscosities between diseased and undiseased sites enables a quantitative determination of the respective relative flow rates; herein, this may be termed differential viscometry or ratiometric viscometry. A clogged catheter may exhibit approximately zero relative flow rate; the measured viscosity may be calculated to be very large, e.g., exceeding 10,000 cP. A clogged catheter is an example of a mathematical singularity wherein approximately infinite viscosity is measured and approximately zero relative flow rate may be calculated or observed; computationally (and practically), an experimentally determined value (e.g., 10,000 cP, 10 μl/sec, etc.) may represent “infinite” viscosity and “zero” absolute flow rate. This example illustrates an approximate “turndown” ratio of 10,000:1 wherein the range of the ratiometric or differential viscometry spans five orders of magnitude.

The examples given for aspirate characteristic included: 6 cP viscosity, Qr=67%, 1cc/sec flow, “clot,” “clog,” “free-flow,” “occlusion score.” The first three of these examples (6 cP viscosity, Qr=67% relative flow rate, 1cc/sec absolute flow rate) are quantitative (variable) data. The first two of these examples (6 cP viscosity, Qr=67% relative flow rate) are intensive properties of the fluid; the third example (1cc/sec absolute flow rate) is an extensive property of the system. The final four example aspirate characteristics (“clot,” “clog,” “free-flow,” “occlusion score”) consist of attribute data (utilized by prior-art) methods to describe flow, flow characteristic, flow state or characteristic of flow. Analysis of variable data (of the present invention) may be utilized to quantitatively measure relative flow rate and/or viscosity as continuous data, whereas attribute data may be utilized to monitor flow for change (e.g., free-flow changes to clog, thrombus changes to clot, etc.). The example measured quantities (viscosity, relative and absolute flow rate) are shown expressed in engineering units (cP, % flow and cc/sec); one or more calibration constants may be invoked in the transformation of pressure data (in units including Pascal, psi, mmHg, bar, etc.) into viscosity and flow data in engineering units. Conversion of variable data (e.g., viscosity, flow rate) into engineering units (e.g., cP, cc/sec) is optional in embodiments of the present invention; the data may be ratiometrically analyzed with or without invoking calibration data.

The embodiment of LCO with aspiration and infusion 400 depicted in FIG. 5A includes infusion pump 460 and infusion tube 320; fluid for infusion is provided through supply tube 480. Some embodiments feature infusion pump 460 comprised of a positive displacement pump such as a piston, syringe, diaphragm pump, peristaltic, etc.; the infusion pressure may range to in excess of approximately 10,000 psi. Some embodiments feature infusion pump 460 comprised of a variable speed, stepper, or servo-motor; infusion pump 460 may have a controlled pressure and/or flow output which may be adjusted/regulated/set by system controller 810 other entities including a clinician. Some embodiments feature infusion pump 460 comprised of a pressurized reservoir or bladder and valve(s); these embodiments being typically non-pulsatile. Non-pulsatile embodiments of infusion pump 460 may be preferred because cyclic reaction forces bearing upon system components including infusion tube 320, coiled infusion tube 475, catheter 220 and/or catheter tip 230 may generate cyclic transverse displacements of components. Cyclic transverse displacements may cause erratic jet direction and/or vascular trauma. Herein, infusion is considered a factor and the flow rate (or pressure) developed is considered a level. Infusion tube 320 is shown to be internal to catheter 220, other embodiments may include infusion tube 320 external to catheter 220. Infusion tube 320 is shown to protrude or extend past the distal end of catheter 220, other embodiments may include a retracted or extended infusion tube 320.

FIG. 5B depicts an oblique, cutaway view of an LCO with infusion and aspiration 400 which includes mechanized positioning of coiled infusion tube 475 by means of infusion tube drive motor 470. As shown in FIG. 5B, the distal terminus of coiled infusion tube 475 is located proximally to manifold 490 such that the lumen of catheter 220 is unoccluded by coiled infusion tube 475 between catheter tip 230 and manifold 490 (where aspirate flow may be diverted through aspiration pump 440 and to waste tube 450). Vascular access 225 delineates intravascular from extracorporeal portions of catheter 220. Coiled infusion tube 475 may be advanced or retracted within and/or external to catheter 220 to act as a hydrodynamic or mechanical lance to perform functions that include: (1) mechanical/hydrodynamic lancing of thrombus 240, (2) mechanical/hydrodynamic lancing of thrombus clogging or corking catheter 220 and/or (3) inject saline into catheter 220 at proximal, distal or intermediate positions therein.

FIG. 5C depicts an oblique, enlarged view of infusion tube 320 (or coiled infusion tube 475) extending distally from catheter tip 230 that depicts a plurality of nozzles (i.e., holes) for liquid discharge; radial nozzle 360 and axial nozzle 340 are shown. Various embodiments of the invention include single or multiple instances of either or both radial nozzle 360 and/or axial nozzle 340; nozzles may be inclined to or offset from any axis to discharge infusion fluid in directions which are combinations of the axial, radial and tangential directions/axes of infusion tube 320. Infusion tube 320 (or coiled infusion tube 475) is shown to extend beyond catheter tip 230 by a distance denoted extension 380. Extension 380 may be a fixed distance; some embodiments of the invention include means to adjust the extension 380. Extension 380 may assume negative values as the infusion tube 320 (475) may be retracted proximally past catheter tip 230 and in the interior of catheter 220; when infusion tube 320 (475) is retracted to negative values of extension 380, the fluid contents of catheter 220 may thereby be modified or altered (e.g., saline flush, etc.). Relative motion between infusion tube 320, 475 and catheter 220 catheter tip 230 may be achieved by manual manipulation of components, or the motion may be mechanized, as depicted in FIG. 5B.

FIG. 5D depicts a partial cutaway view of the distal (catheter 220) portion of an LCO with infusion and aspiration 400, deployed within vessel wall 280. Infusion tube 320 extends from catheter tip 230 by (positive) distance extension 380. Infusion fluid discharged from radial nozzle and axial nozzle (denoted in FIG. 5C) generates radial jet 560 and axial jet 580. Radial jet 560 and axial jet 580 are shown in the direction of flow; equal and opposite reaction forces are exerted upon infusion tube 320 and supporting or surrounding structures (e.g., catheter, vasculature, other solid surface, etc.). Axial jet 580 bears proximally upon the distal end of infusion tube 320; a limited amount of motion in the axial direction typically occurs because of the column stiffness of catheter 220. Radial jet 560 radially bears upon infusion tube 320 in a direction shown to be transverse or perpendicular to the axis of catheter 220. Consequently, a downward radial force is exerted upon infusion tube 320 and catheter tip 230; radial jet 560 acts to force catheter tip 230 in the downward direction such that catheter 220 and/or catheter tip 230 is/are pressed into contact with vessel wall 280.

FIG. 5E depicts a similar view to FIG. 5D except that extension 380 is increased such that infusion tube 320 extends to overlap thrombus 240 and/or attachment 260. Radial jet 560 is shown to directly impinge upon and penetrate into thrombus 240; axial jet 580 is shown to impinge upon and penetrate attachment 260. The configuration of FIG. 5E may be advantageous because: (1) infusion tube 320 is in close proximity to or in contact with vessel wall 280, and (2) radial jet 560 directly impinges upon thrombus 240 to erode, macerate, displace or disintegrate it. An advantage of infusion tube 320 being in contact with or close proximity to vessel wall 280 is that axial jet 580 is directed parallel to the surface of vessel wall 280. At higher infusion pressures, either radial jet 560 or axial jet 580 may be capable of inflicting vascular trauma including laceration, perforation, abrasion or inflammatory response in the vessel wall or other tissue. Radial jet 580 acts to acts to keep infusion tube pressed downward/radially outward such that the distance between radial jet 340/radial jet 560 and the upper surface of vessel wall 280 is hydrodynamically forced to a location that may be at or near maximum given the anatomical constraints of the patient. A non-pulsatile or minimally pulsatile infusion pump 460 system may be preferred to minimize cyclic transverse deformations or vibrations (i.e., up and down in FIG. 5C and FIG. 5D). Transverse vibration may cause significant transverse deflection, which may impact vessel wall 280. Embodiments featuring non-pulsatile infusion pressure (e.g., non-isovolumetric pressurized reservoir or bladder, etc.) may be preferred for an infusion supply system.

A visible difference between FIG. 5D and FIG. 5E is the change in extension 380; infusion tube 320 is shown extended distally in FIG. 5E with respect to FIG. 5D. The physical act of changing extension 380 may enable infusion tube 320 to act as a lance, obturator or probe to pierce, penetrate, scise, abrade or otherwise mechanically interrupt attachment 260 and/or thrombus 240 by means of mechanical and/or hydrodynamic effects. This action may have different effects in the presence or absence of radial jet 560 which may hydrodynamically force infusion tube downward to be in contact with or close proximity to the lower surface of vessel wall 280. Axial jet 580 is shown to be optimally positioned, in contact with or close proximity to vessel wall 280. Axial jet 580 may be therefore oriented to be parallel to vessel wall 280 such that vascular trauma may be kept to a minimum while hydrodynamically eroding thrombus 240 or attachment 260 at the contact interface with vessel wall 280. When infusion tube 380 is pressurized, fluid is discharged at a rate which may be proportional to the pressure (or flow rate) and inversely proportional to the diameters of axial nozzle 340 and/or radial nozzle 360. A larger-diameter nozzle discharges greater mass flow at lower velocities; a smaller-diameter nozzle discharges lesser mass flow at higher velocities. Optimized ratios of the position, diameter and direction of any nozzle may be experimentally determined for optimized ratio of radial jet 560 and axial jet 580 dimensions for any infusion pump 460 embodiment, system, configuration, operating pressure or flow rate.

An objective of any catheter-based thrombectomy procedure is to aspirate or extract thrombus through a catheter; therefore the successful procedure includes the aspiration of blood and tissue components that are of viscosity greater than that of blood. Throughout a thrombectomy procedure, any measured value of, or increase in, aspirate viscosity (above that of blood), may be indicative of effective therapy being administered; this may arise from the execution of a deterministic event such as an efficacious thrombectomy operating mode. This is in contrast to ineffective therapy which may arise from deterministic events or clinical conditions including: catheter deployed in a healthy (non-thrombotic) location, system process variables (factors/levels) improperly adjusted for thrombus morphology, a rigid/firmly attached thrombus, etc. During periods of a thrombectomy procedure wherein the viscosity of the aspirate is near that of blood, ineffective treatment is being administered; this is detrimental treatment as viable blood is aspirated which may limit the procedure duration, thoroughness and overall efficacy. Embodiments of the present invention systematically execute multiple thrombectomy operating modes to identify and exploit efficacious thrombectomy operating modes in a minimum amount of time and with a minimum amount of loss of healthy blood.

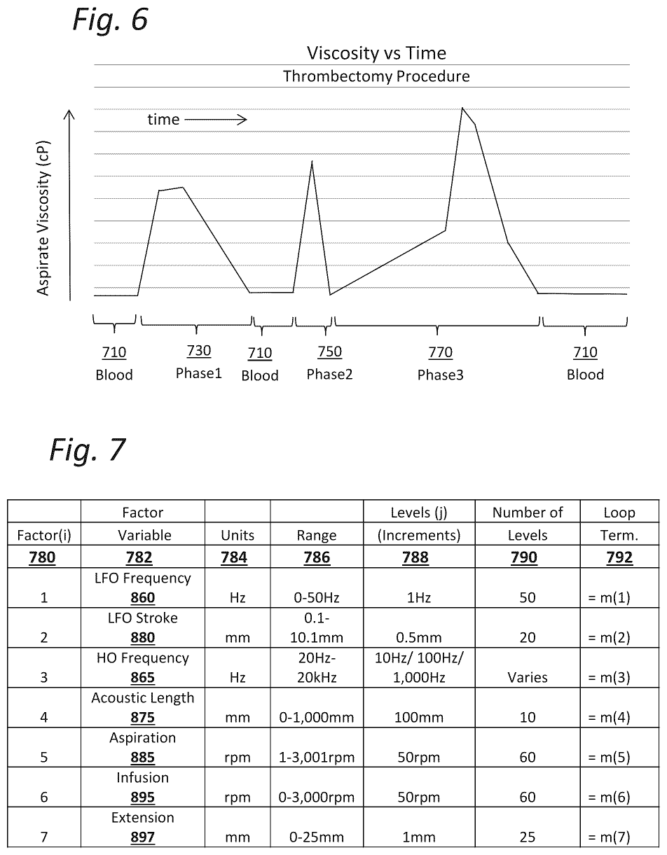

FIG. 6 depicts a graph Viscosity vs. Time (Thrombectomy Procedure) which may be representative of any portion of a thrombectomy procedure which comprises viscometric aspirate analysis or viscometric sampling. FIG. 6 may be segmented into phases: blood 710 (appears 3 times), phase 1 730, phase 2 750, and phase 3 770. Each of the enumerated phases are responses to deterministic events (e.g., thrombectomy operating mode, repositioning of catheter 220, etc.); responses to deterministic events include phenomena such as: entrainment of thrombus 240, disintegration of thrombus 240, dislodgement of thrombus 240 from vessel wall 280 or any other process by which thrombus 240 is aspirated into catheter 220. FIG. 6 depicts an initial viscosity which may be inferred to be that of blood 710; subsequently, phase 1 730 is shown to exhibit a rapid increase in viscosity (or % thrombus in aspirate, or decrease in flow rate, etc.), followed by a leveling off, and followed by a slower decline in viscosity. Each of the depicted phases (e.g., phase 1 730, phase 2 750, and phase 3 770, etc.) has undergone any or all of the following deterministic events: (1) efficacious positioning of catheter tip to thrombus distance 250 by viscometric distance sampling (e.g., distance subroutine 1050, etc.), (2) viscometric determination a first efficacious thrombectomy operating mode, (3) determination of optimal aspiration/infusion rate or other system factors and levels (e.g., effect subroutine 1000, etc.), by system controller 810. Phase 1 730 may be inferred to have identified an effective or preferred combination of factors and levels whereby thrombus 240, attachment 260 and/or vessel wall 280 are disrupted which causes disintegration of one or more attachments 260 such that thrombotic material is released and aspirated. The viscosity of phase 1 730 is shown to diminish as the effectiveness of the identified thrombectomy operating mode is diminished or exhausted. Phase 1 730 is followed by another period of blood 710; during this time, deterministic events including changes to system process variables (factors and/or levels); device placement may be changed (by means such as distance subroutine 1050, etc.). The onset of phase 2 750 may be inferred to detect a second preferred combination of factors and levels which is characterized by a rapid increase and decrease in viscosity. Phase 2 750 may be illustrative of the disintegration and aspiration of a small thrombus 240 or subcomponent thereof. Phase 3 770 is illustrative of a gradual increase in viscosity which may be indicative of approaching a preferred frequency or efficacious thrombectomy operating mode, followed by a rapid increase in viscosity which may be indicative that a preferred frequency or efficacious thrombectomy operating mode has been identified. A gradual decline in viscosity just after the peak may be indicative of a decreasing amount of thrombotic material in the aspirate, such as may occur when an eroded or vibrating mass of thrombus 240 is depleted and has been aspirated. A more rapid decline in viscosity may be indicative that one or more factors and/or levels are not in an efficacious range. Subsequent to phase 3 770, the aspirate viscosity returns to that of blood 710; this may mark a procedure endpoint as one, two or three thrombi 240 may have been aspirated, or a compound-type thrombus 240 may have been disintegrated and aspirated in three distinct phases.

The abscissa of FIG. 6 is shown as time, this is consistent with prior art and the figures presented heretofore in this disclosure; however, in FIG. 6, the abscissa is subdivided into attribute data regions or phases (blood 710, phase 1 730, phase 2 750, phase 3 770). The graphical representation of FIG. 6 illustrates a distinction of the invention over prior art; some embodiments of the invention correlate measured (or monitored) system response (e.g., aspirate viscosity, pressure, flow, etc.) to deterministic events (e.g., thrombectomy operating modes, catheter positioning, combination of system factors and levels, etc.). In the prior art, measured responses are, at most, correlated to the passage of time. In prior art, measurement data are converted to attribute data and the attribute data are monitored for a change of state (e.g., flow state). Upon detection of a change of state, prior art fails to provide any quantitative description of the control response, examples of non-qualitative control responses include open/close a valve, adjust a pump, etc. The prior art is limited to monitoring for a detectable change, and then reacting to the detecting by opening or closing valves or adjusting a pump. Conversely, some embodiments of the invention execute deterministic events, and measure the system response in quantitative terms; a system response that is relevant in a thrombectomy procedure is the amount of thrombus that is contained within the catheter at any time. If there is no thrombus in the catheter, the thrombectomy efficacy is zero; an inference from this data is to change a system variable (e.g., catheter position, suction level, etc.). If there is too much thrombus in the catheter (e.g., clog or corking), the thrombectomy efficacy is also zero, because there is no flow. A first inference from this data is that aspiration may have been adjusted to an improper setpoint; this may be addressed by software firmware updates. A second inference from this data is that anti-clogging or anti-corking means (e.g., impulse mechanism 90, LCO 100, etc.) be implemented.

Embodiments of a representative LCO/HO thrombectomy system with aspiration and infusion 400 may comprise any plurality of multiple independent systems (factors) including (as examples): (1) variable-frequency LF oscillator, (2) variable LF oscillator stroke (e.g., piston in cylinder, tube compression, peristaltic pump oscillation, etc.), (3) variable high frequency, small amplitude sonic transducer (HO, including sonic transceiver), (4) variable length acoustic tube, (5) variable infusion tube extension, (6) variable viscometric aspiration and (7) variable infusion rates; each independent system may be termed a process variable or factor. Any number of these independent systems (factors) may be selected to operational depending upon factors including: clinical indication, catheter specific factors or features, clinician preference, etc. Typically, each factor has a plurality of setpoints (levels) that may be updated (whereupon an “updated,” “new” or “different” and/or “repeated” setpoint combination is selected); this happening a plurality of times during the course of a thrombectomy procedure. The thrombectomy system setpoints (factors and levels) may be manually adjusted by a clinician; some embodiments employ feedback and/or thrombectomy control flowcharts or algorithms to adjust or update one or more setpoints (levels) in response to measurement data (e.g., viscosity, relative flow rate, % thrombus concentration, etc.). Catheter-based thrombectomy systems may utilize a clinician to select/identify/target one or more thrombi (including through the utilization of fluoroscopy, IVUS, or other imaging system) and advance a catheter into proximity; thereafter, manual and/or automated processes may be executed by the thrombectomy system and/or clinician to provide suction, aspiration, infusion, mechanical or hydrodynamic maceration, or other methodologies to the target site. Setpoint/level control is automated in some embodiments; clinician input may also comprise one or more feedback inputs in the determination of setpoint/level control of each factor (or adjustable system process variable).

Embodiments of LCO 100 thrombectomy systems including LCO with aspiration and infusion 400 comprise a control system that updates any or all system factors, process variables, setpoints/levels (e.g., frequency, stroke, acoustic tube length, aspiration, infusion, etc.) by means of feedback from one or more measuring instruments (e.g., viscometer, pressure transducer, sonic receiver, flowmeter, accelerometer, etc.). An objective of a catheter-based thrombectomy system is to aspirate fluid that is measured to exhibit viscosity greater than a baseline value of viable blood (at approximately 4 cP); therefore aspirate viscosity comprises an example feedback input for updating system process variables (factors) and/or setpoints (levels) in some embodiments of the present invention. Some embodiments comprise feedback input from data sources including: pressure transducers, flowmeters (e.g., Coriolis, ultrasonic, etc.), sonic receivers, motor current measurements, clinician input, accelerometers, etc. Co-pending applications describe the methods and mathematical transformation of pressure data (e.g., from a pressure transducer, in units of pressure) to viscometric data (in units of viscosity, e.g, cP, Stokes, Pa's, etc.) in time domain. Herein the terms pressure data, viscometric data, flow data, % thrombus data, thrombus load, etc. may be used to describe quantitative data which may be implemented as a feedback mechanism in embodiments of the present invention. Viscometric measurements, as implemented by the invention and references, comprise pressure measurements and further comprise methods or algorithms for the transformation of pressure data into measurements of viscosity, relative flow rate, % thrombus concentration or similar derived or calculated unit or measurement. Some methods and measurements of the invention comprise determination of quantitative descriptors of intensive physical properties of the aspirate; this is in contrast to prior art embodiments that monitor extensive system properties including “flow state,” “characteristic of flow,” “occlusion score,” “clot,” “free-flow,” etc.

The example thrombectomy control algorithms, flowcharts, methodologies and/or strategies (presented herein as embodiments) illustrate distinctions of some embodiments of the present invention: system control over a plurality of system factors and/or setpoints/levels. In some embodiments, this is accomplished by algorithms executing algebraic operations upon variable data from quantitative instrumentation comprising the thrombectomy system. This is in contrast to prior art, wherein variable data (e.g., pressure, differential pressure, etc.) are first assigned an attribute (e.g., clot, free-flow, 0, 1, 2, occlusion score=3, etc.) and then the attribute states are counted or monitored for change. Furthermore, in methodologies of the prior art, any event of a “change of state,” or “change of characteristic,” or “change of score,” may only be correlated to the passage of time. Methodologies of the invention include correlation of measurement data (of physical properties of the aspirate) to deterministic events that predicated the value returned as measurement. The scope of the invention is encompassing of the philosophy that some intensive physical properties of aspirate (e.g., viscosity, % thrombus, relative flow rate, etc.) are predicated upon deterministic events (instead of random events or the passage of time). In other words, the measured physical properties of the aspirate change with time; prior to the measurement, the sequence of deterministic events is herein identified as the cause of the physical property state that is subsequently measured. An example methodology is to correlate a physical property measurement to time and correlate one or more deterministic events to the same time scale; measurement data of a physical property may be correlated to predicating deterministic events.

FIG. 7 depicts a representative list of example factors 780 identified by an integer (the factor loop index, i) for computational efficiency; factor variable 782 describes each factor in words (e.g., LFO frequency 860, LFO stroke 880, HO frequency 865, acoustic length 875, aspiration 885, infusion 895, and extension 897, etc.). The level 788 of each factor variable 782 may be expressed with units 784 (e.g., Hz, mm, rpm, on/off, etc.) and may be experimentally/procedurally evaluated within a range 786 of levels 788. The combination of range 786 and levels 786 enables calculation of a number of levels 790 such that a loop termination 792 index (an integer) is determined. For example: when factor loop index (i) is equal to 1 (i=1), factor 780 is shown defined to be LFO frequency 860; LFO frequency 860 may be incremented (50 times) from 0 Hz to 50 Hz in 1 Hz increments. When level loop index (j) is equal to 50 (j=50), the loop is terminated because loop termination index 792, m(i=1)=50. Similarly, when factor loop index (i) is equal to 2 (i=2), factor(i) 780 is shown defined to be LFO stroke 880; LFO stroke 880 may be incremented (20 times) between 0.1 mm and 10.1 mm in 0.5 mm increments. When level loop index (j) is equal to 20 (j=20), the loop is terminated because loop termination index 792, m(i=2)=20. FIG. 7 is shown constructed in a manner convenient for programming in any selected computer language by persons of ordinary skill in computer programming.

Some embodiments of the present invention comprise a thrombectomy system featuring a number of independent system process variables (factors) under setpoint (level) control by any entities (or combination thereof) including: system controller 810, database/compiler 2020 and/or a clinician, etc.. FIG. 6 depicts three representative viscosity vs. time “waveforms” or “phases” (of a thrombectomy procedure). Example phase 1 730 depicts a rapid increase in measured viscosity; this may result from many causes including a vibrational response at LFO frequency 860 at a level (e.g., 25 Hz, 2 Hz, 17 Hz, etc.), or a combination such as: LFO frequency 860 level and LFO stroke 880 level (e.g., 0.5 mm, 4 mm, 0.25 mm, 3 mm, etc.) along with other factors/levels may provide efficacious thrombus aspiration. Some embodiments of the present invention may “sweep” a plurality of levels of one or more factors, such as by incrementally or continuously increasing/decreasing the speed of motor 180 or the frequency of a sonic transducer 110. Phase 1 730 of FIG. 6 may arise from incrementing LFO frequency 860 from 0 Hz to 50 Hz at 1 Hz increments; LFO frequency 860 may be defined as factor(1) or factor1. Factor(1) may be incremented (a representative) 50 times throughout this range, indicated by number of levels 790. A computer loop is well suited for this repetitive task; this loop may be a FOR/NEXT, a DO, or a DO WHILE loop, etc. as provided by the programming language. Phase 1 730 may have initiated at 25 Hz setpoint/level of factor(1); a DO WHILE (e.g., viscosity>4 cP, slope>0, etc.) may thereby “hold” factor(1) at 25 Hz level while the viscosity increases and decreases as shown in FIG. 7A. In FIG. 7A, any portion of the graph where the viscosity is greater than that of blood 710 is indicative of effective therapy being delivered. Some embodiments may “hold” factor(1) (e.g., at 25 Hz, 5 Hz, 40 Hz, etc.) while incrementing/decrementing the levels 788 of other factors(i≠1).

In some embodiments, loops (including nested loops) may vary any or all of the remaining factor variables 782 while any factor (e.g., frequency) remains at a fixed level (e.g., 25 Hz, 5 Hz, 1 Hz, etc.); objectives of any such loop may be to identify thrombectomy operating modes which exhibit an increase or maintenance of the viscosity of the aspirate above that of blood 710. A positive viscosity slope (viscosity is increasing) may be indicative of increasing therapeutic treatment, a negative slope being indicative of the diminishing thrombus in the aspirate; however, some embodiments may comprise an algorithm which “waits” for the viscosity to return to that of blood 710. In some embodiments, as any loop terminates, a successive loop may be executed; each loop may terminate as the loop termination index 792 reaches the value of m(i), or control may be diverted by means of conditional statements such as IF (condition) THEN NEXT i.

FIG. 8 depicts an example embodiment of system controller 810, with inputs (shown above) and outputs (shown below); each output may have a setpoint (e.g., factor(1) 780 is set to a level 788 of 25 Hz, factor(2) 780 is set to a level 788 of 1 mm, etc.) assigned by system controller 810. FIG. 8 depicts eight example outputs: LFO frequency 860, LFO stroke 880, aspirate pump 440, infusion pump 460, acoustic tube length 130, extension length 380, HO frequency 865, and clinician data 890. A plurality of these outputs may be identified by factor 780 and a level 788 which may be assigned by entities including system controller 810 or clinician input 895. Three example inputs are shown in FIG. 8: pressure transducer 420, sonic transducer 110 and clinician input 895, which may include data from accelerometer 410. The methods and mathematical transformation of pressure data from pressure transducer 420 into viscometric or quantitative flow data is presented elsewhere in the references. Other inputs to system controller 810 may comprise flowmeters, conductivity meters, spectrometers, etc. Clinician data 890 may comprise analog or digital data (e.g., viscosity, flow, slope, setpoints, etc.) which may comprise display monitor, audio speaker, cellular phone or other remote console, etc.; clinician input 895 may comprise any of: accelerometer 410, joystick, mouse, keypad, footpedal, knobs, etc. Clinician data 890 may comprise data stored as thrombectomy procedure log file 1300. System controller 810 is presented in further detail in conjunction with FIG. 15.

FIG. 9A depicts a representative thrombectomy control flowchart 901 (algorithm) as may be utilized by system controller 810 or other thrombectomy control system embodiment. Start 905 may comprise any clinical preparations including power-up self-tests, catheter purge, calibrations, etc. Initialize setpoints 910 may include calculation/measurement/calibration of blood viscosity and assigning that value to V_{0,0}, initializing the levels for factors such as: acoustic tube length 130, extension length 380, LFO stroke 480, etc. Position catheter 920 may be executed by the clinician; data from accelerometer 410 may be collected, stored and/or correlated to any or all subsequent steps. Distance subroutine 1050 (FIG. 9C) is invoked to infer the distance between the catheter tip and thrombus. If the measured or inferred distance is “too large,” (e.g., by a threshold arbitrary constant δ₁, etc), then Advance Catheter 944 is executed by manual or automated means. If the measured or inferred distance is negative or “too small,” (e.g., by a threshold arbitrary constant δ₂, etc.), then Retract Catheter 948 is executed by manual or automated means. Position Catheter 920 and Distance Subroutine 1050 represent an example set of machine instructions to obtain proper catheter positioning prior to further experimentation to ablate and aspirate thrombus. Distance Subroutine 1050 invokes Viscosity/Slope Subroutine 1070 (FIG. 9E); therein, methods such as Time-Domain Viscometry (as disclosed in the references) are employed to measure the viscosity and slope of the aspirate. With the catheter 220 in position with respect to thrombus 240, a sequence of experiments is conducted utilizing a representative computer loop structure of FIG. 9A. In FIG. 9A through FIG. 9D, the measured viscosity at loop indices i and j, is denoted as V(i,j), the measured slope is denoted as S(i,j). In some embodiments, a plurality of values of V(i,j) and S(i,j) are stored in memory, in some embodiments a list of efficacious values of i and j are compiled (e.g., by sorting) and stored in a file of “preferred values.” These preferred values may be used subsequently in the procedure (e.g., by assigning loop indices to be from the preferred values file). A preferred values file is, for example, comprised of loop index values (i and j) that positively correlate to thrombectomy efficacy or the measured amount of thrombus in the catheter. The extension to any number of factors 780, levels 788, multiple independent loops, nested loop levels (with indices such as i, j, k, l, m, n, etc.), etc. is straightforward and anticipated.

FIG. 9C depicts representative distance subroutine 1050 that utilizes measured viscometric data to infer catheter tip to thrombus distance 250 and invoke an appropriate control response by system controller 810. Distance subroutine 1050 is so named because an objective is to enact or enable a clinician to reposition catheter 220 such that catheter tip 230 is positioned for improved thrombectomy efficacy. Distance subroutine 1050 invokes Viscosity/Slope Subroutine 1070 to be executed and return the measured viscosity value, V(i,j); this value is algebraically compared to known values and/or arbitrary constants such as the viscosity of blood, V₀, and prermined threshold values, δ₁ and δ₂, etc.. Aspirate samples that yield measured aspirate viscosity less than δ₁V₀ infer that the catheter tip to thrombus distance 250 too large for efficacious aspiration of thrombus 240 without unnecessary blood loss. The arbitrary constant, δ₁ is predetermined experimentally and typically lies in the approximate range of 1.00<δ₁<2.00, and V₀ is the measured viscosity of blood (≈4 cP). Catheter 220 is advanced into closer proximity to thrombus 240 by manual manipulation or automated methods; manual manipulation may be facilitated by audiovisual communication to clinician such as “ADVANCE CATHETER” being played over an audio speaker.

Aspirate samples that yield measured aspirate viscosity greater than δ₂V₀ infer that the catheter tip to thrombus distance 250 is negative or too small for efficacious aspiration of thrombus 240 without excessive risk of clogging or corking catheter 220. The arbitrary constant, δ₂, is experimentally predetermined and typically lies in the approximate range of δ₂>30. Catheter 220 is retracted to farther proximity from thrombus 240 by manual manipulation or automated methods; manual manipulation may be facilitated by audiovisual communication to clinician such as “RETRACT CATHETER” being played over an audio speaker.

For example, by selecting representative values of δ₁=1.1 and δ₂=50, the control response of distance subroutine 1050 is depicted to be: IF the measured aspirate viscosity is less than ≈4.4 cP (ratiometrically compared to prior values of the patient blood assumed to be ≈4.0 cP) THEN “ADVANCE CATHETER,” and IF the measured aspirate viscosity is greater than ≈1,000 cP THEN “RETRACT CATHETER.” Thus, catheter tip 230 may be manipulated and/or positioned to an optimum position, and control is returned to factor loop 930 of thrombectomy control flowchart 901.

Among the functionalities of representative thrombectomy control flowchart 910 is to sequentially execute a plurality of experiments in thrombus aspiration, each experiment comprising operation of the apparatus and measurement of the aspirate viscosity. Typically, experiments may be conducted in approximately 1 second (range of approximately 0.2 s to 5 s). Thrombectomy control flowchart 901 depicts a representative computer loop structure, wherein loop indices (e.g, i, j, k, l, etc.) are used to incrementally increment and/or decrement setpoint values (levels 788) of system parameters (factors 780).

FOR—NEXT factor loop 930 is an outer loop to sequentially increment (and thereby select) each of the n factors; in the example embodiment presented herein, n=7. FIG. 7 depicts representative factor variables 782, range 786, increment 788 and loop termination index 792 corresponding to each factor 780. During the first execution of FOR—NEXT factor loop 930, i=1; when i=1 the levels 788 of factor 780 (LFO Frequency 860) are incremented. With each increment (or change) to the apparatus setpoints, Operate Apparatus 902 is executed, wherein the systems (e.g., LCO 100, aspirate pump 420, infusion pump 460, etc.) of the apparatus are activated or “turned on.” Viscosity/Slope Subroutine 1070 (FIG. 9E) is invoked to measure the viscosity of the aspirate. In this manner, an experiment is conducted (with factor 780 and levels 788 assigned by i and j loop indices), and a physical property (e.g., viscosity) of the aspirate is measured.

Referencing FIG. 7, nested FOR—NEXT level loop 940 increments the level 788 of LFO frequency 860 (when i=1) from 0 Hz to 50 Hz in 1 Hz increments as j is incremented from 1 to 50. At each j value of level loop 940, Operate Apparatus 902 and measure viscosity and slope 950 are executed. After each experiment, control is transferred to effect subroutine 1000; a representative example subroutine is shown in FIG. 9B. In other embodiments, loop indices may be decremented (e.g., FOR j=n to 0 STEP −1) or a loop index may be changed, updated or reassigned by a command line. As examples, decrementing loop indices results in a “reverse sweep” which ranges from higher levels to lower levels of a factor; statements such as “j=j−5” may result in repeating the five (5) prior levels 788 of any factor 780, which is defined by the factor loop 930 index, i. As depicted for illustration purposes, FIG. 9A is a nested loop with two loop indices (i and j); this abridged flowchart does not generate full factorial, fractional or other DOE matrix for the representative LCO 100 embodiment which features seven independent subsystems. Additional nested loops and loop indices (e.g, k, l, m, n, etc.) are required to generate more complete experimentation within the matrix of combinations of factors and levels. FIG. 9A through FIG. 9E are abridged for brevity and clarity; however, these figures illustrate (1) a set of machine instructions for executing tasks (e.g., Operate Apparatus 902, etc.), (2) a method of invoking subroutines to execute tasks (e.g., Measure Viscosity/Slope 1070, Operate Impulse Mechanism 90, etc.), and (3) a method to evaluate conditional statements to adapt future experiments for efficacious thrombectomy efficacy.

Representative effect subroutine 1000, depicted in FIG. 9B, and so named because the subroutine analyzes the “effect” of a previously executed “cause” (e.g., thrombectomy operating mode, combination of factors and levels, clinician manipulation, etc.). Effect Subroutine 1000 invokes Clog Detect/Avert Subroutine 1060 (FIG. 9D); if this subroutine detects that the viscosity, V(i,j), exceeds arbitrary constant V_{clog}, then countermeasures are invoked. The example countermeasures include impulse mechanism 90, LCO 100, infusion pump 460, etc.

Effect subroutine 1000 numerically analyzes data by executing conditional statements and determines one or more subsequent steps or thrombectomy operating modes to be executed. Effect subroutine 1000 is shown to execute representative conditional IF—THEN (THEN may be omitted by allowable BASIC syntax and for compactness) statements with viscosity and/or slope as (variable data) arguments. Other conditional statements and arguments may comprise other generalized subroutines. Effect subroutine 1000 is shown such that control may be transferred to various locations within thrombectomy control flowchart 901 depending upon the magnitude and/or slope of aspirate viscosity or other system measurement, parameter and/or clinician input. Returning control to level loop 940 resets j to j=1 and the sweep (of the levels 788 of the selected factor 780) is repeated; returning control to measure viscosity and slope 950 re-measures viscosity and slope after a predetermined dwell time. Returning control to NEXT Level 970 continues the sweep of factor 780. These example thrombectomy control flowchart 901, effect subroutine 1000 are representative of any thrombectomy control algorithm to continuously or incrementally vary any or all of the system parameters (e.g., factors and levels). Incrementing, decrementing or otherwise changing loop index (i) changes the factor 780 (e.g., factor variable 782 is equal to: LFO Stroke 880 when i=2, HO Frequency 865 when i=3, acoustic length 875 when i=4, etc.). Effect subroutine 1000 is shown to be comprised of predetermined dwell times and conditional statements (IF-THEN statements), in general, subroutines may be comprised of loops (FOR—NEXT, DO, DO WHILE, etc. loops) and nested subroutines. Dwell times (D0, D1, D2, . . . Dₗₐₛₜ) may be experimentally determined; if a dwell time is longer or shorter than required, the procedure efficacy and/or duration may be adversely affected. In some embodiments arguments of conditional statements include quantitative or qualitative system parameters such as: flow, flow rate, flow state, characteristic of flow, etc.

The representative conditional IF-THEN statements (of example effect subroutine 1000) are used to compare the measured value and/or slope of the aspirate viscosity to previous iterations (i.e., thrombectomy operating modes or experiments) and/or other predetermined values. If the measured viscosity of the aspirate is within the range of 4 cP<V(i,j)<V_{clog}, then the thrombectomy system may be inferred to be operating in a desired regime of extracting thrombus. Predetermined dwell times (e.g., D0, D1, D2, . . . ) may be executed prior to returning control to a location within thrombectomy control flowchart 901. Likewise if the slope of the viscosity is increasing {i.e., S(i,j)>S(i,j−1)} then the thrombectomy system may be inferred to be operating in a desired regime of extracting thrombus and that the concentration or composition quality of thrombus in the aspirate is increasing. The duration of dwell may be a function of the slope (e.g., IF {S(i,j)>S(i,j−1)} THEN DWELL S(i,j)×D1); this may provide a modified dwell in response to an increasing slope. The ratio of slopes may be compared to an arbitrary, experimentally determined constant, κ, {S(i,j)/S(i,j−1)>κ} so that the dwell or other system parameters may be altered if the ratio of slopes does or does not exceed any threshold value. The arbitrary or experimentally determined constant, K, typically is in the range of 1.0<κ<5.0; if the slope, S(i,j) increases by greater than a factor of five between successive measurements a clog or impending clog may be inferred, this may require manual or automated clog countermeasures.

“Dwell” may be comprised of any or all of the following: (1) all levels 788 (setpoints) are retained for a specific period of time (e.g., D0=1s, D1=5s, etc.), (2) all levels 788 are retained for a variable amount of time (e.g., DWELL (S(i,j)×D0), DWELL (κS(i,j)×D1, etc.), and/or (3) one or more levels 788 may be independently incremented or decremented during the “dwell.” The latter (case 3) gives rise to additional subroutines which may invoke one or more loops using additional loop indices (e.g., k, l, m, etc.) to increment or decrement the levels 788 of one or more of factor variable 782 such as factor 780 (e.g., 5, 6, 7, etc.). Such a subroutine may sweep the levels of factor variable 782; for example, aspiration 885, factor(i=5) 780 levels 788 may be incremented or decremented throughout all or a portion of range 786 under loop index k while maintaining loop indices i and j constant. The example number of factor variables 782 is shown to be 7; therefore nested loops (e.g., 2, 3, 4, 5, 6, or 7 deep, etc.) including parallel nested loops also may comprise the present invention.

Some embodiments of the knowledge-based thrombectomy system comprise a control algorithm (e.g., thrombectomy control flowchart 901) which causes operation of the apparatus and increments, decrements or otherwise “sweeps” the levels 788 of one or more factor variables 782 (e.g., LFO frequency 860, HO frequency 865, etc.) while other factor variables 782 may be held constant (e.g, at initial, previous, nominal or zero values). The viscosity of the aspirate is measured for quantitative presence of thrombus; an efficacious combination of factor variables 782 may thereby be identified by a measured value of (or increase in) aspirate viscosity greater than that of blood, but below that of V_{clog}. Some embodiments of the invention may then hold constant that combination of factor variables 782 and levels 788 while any or all remaining factor variables 782 (e.g., aspiration 885, infusion 895, extension 897, etc.) are independently explored, incremented, decremented or swept. The measured viscosity of the aspirate intermittently or continuously provides feedback data which is evaluated as arguments of conditional statements (e.g., IF-THEN, DO WHILE, DO, etc.); the outcome of the conditional statements is utilized to update the setpoint of factor 780 and/or level 788 (e.g., increment, decrement, reset, increase, maintain, decrease, discontinue, repeat, etc.). For compactness, a single nested loop (with loop indices i and j) is shown in representative thrombectomy control flowchart 901, the extension to multiple nested loops with a greater number of loop indices (e.g., k, l, m, n, etc.) may be features of other embodiments of the present invention.

These examples are specific in that the number (n) of factors 780 is chosen to be seven; more generally, any number of factors 780 may arbitrarily be selected in other embodiments depending upon the system/catheter features and clinical indications. Similarly, viscosity and/or slope is/are chosen as the example of measurement data which may be employed as arguments to conditional statements (IF, WHILE, UNTIL, . . . ). Other embodiments may collect input data from sonic transducer 110 to determine the frequency and magnitude of any vibrational modes existing within the thrombectomy system or the patient vasculature. Other embodiments may collect data from other measurement technologies (e.g., flowmeter, conductivity meter, optical meters, spectrometers, etc.). The data utilized in the representative flowcharts are variable data, namely viscosity and slope; in some embodiments, attribute data (e.g., characteristic of flow, flow state, aspirate characteristic, etc.) are utilized as arguments in conditional statements. Different or multiple arguments (of conditional statements, e.g., IF—THEN, DO—WHILE, etc.) may arise in other embodiments not presented herein. Aspects of the present invention comprise the selection of one or more factor variables 782, selecting an appropriate range 786 for each, and exploring any or all factor variables 782 at a plurality of levels 788 while measuring the aspirate for thrombus. The listed examples of factor variable 782 and level 788 are presented in conjunction with the disclosure of embodiments including: LFO frequency 860, LFO stroke 880, HO frequency 865, acoustic length 875, aspiration 885 and extension 897. Some embodiments exercise control over different system parameters such as valves, regulators, and effect changes such as: open a valve, close a valve, increase a pressure, etc.

Embodiments of the present invention invoke the physics of a forced system of mass, spring and damper, wherein an example forcing function may be an oscillating or reciprocating surface. The fluid mass of the system generally comprises the mass of the fluid within cylinder 200, catheter 220 and in the localized bloodstream, generally bounded by vessel wall 280. Elasticity of components, including vessel wall 280, may provide a spring component while damping may be provided by fluid viscosity. Example reciprocating surfaces include the face of piston 120 and/or a corresponding structure of sonic transducer 110, such as a reciprocating piston, vibrating diaphragm, pinch valve, roller pump, etc. Some example reciprocating structures induce oscillatory motion of matter (fluids and solids); this motion may be harmonic, off harmonic or anharmonic. Oscillatory motion of fluid generally within catheter 220 may be of any combination of low, medium or high frequencies within the range of 0.1 Hz to approximately 19,000 Hz (subsonic and sonic ranges). At lower frequencies, a finite, reciprocating fluid displacement may exist; at higher frequencies a series of fluid compressions and expansions may result in infinitesimal displacements. A net inflow or outflow may exist such that the fluid oscillates but does not change direction; in all such cases, the fluid motion is oscillatory. The presented embodiments describe discrete increases (increments) in setpoint values (levels); continuous variation of setpoint values is anticipated by the present invention.

An objective of the present invention is to attrite, disintegrate or dislodge matter that is in contact with or adhered to a wall of a fluid reservoir (e.g., patient vascular system, bloodstream, pipe, tube, tank, etc.) and to subsequently aspirate the matter. Embodiments of the present invention comprise a catheter designed for deployment within a vein or artery, thus creating a system that may be described as a conduit (catheter, etc.) in fluid communication with a reservoir (the bloodstream). Fluid may be discharged from the catheter tip at velocity sufficient to induce characteristic fluid motion within the reservoir (e.g., blood vessel, pipe, tank, etc.). In a “large” reservoir, such as where the characteristic reservoir dimension (e.g., diameter, width, height, etc.) is greater than approximately 10 times the diameter of the catheter, the jet of fluid discharged from the catheter tip will be dispersed and dissipated within a few catheter diameters.

In some embodiments, the present invention comprises the utilization of oscillatory fluid motion to perform “action at a distance” by intermittently discharging liquid into a surrounding environment of characteristic topology. Depending upon the target vascular site, the vasculature topology may be contracting, expanding or constant-diameter; the diameter of target vascular site may range from 1 to approximately 50 times the diameter of the catheter. In some applications, the catheter and vessel diameter may be approximately equal. In cases wherein the catheter and vascular diameters are approximately equal, the vascular flow regime may be dominated by LCO 100 and the “action” (dislodging a thrombus) may be carried out over a greater “distance” (between the catheter tip and the thrombus). The LCO thrombectomy system may thereby provide treatment access to anatomical locations previously unreachable due to native or diseased vascular contractions, constrictions, tortuosity, etc. Partial or total occlusions, which are distal to the catheter tip and otherwise inaccessible by other devices, may thereby be disrupted, dislodged or disintegrated at a greater distance than that which may be attained by prior art.

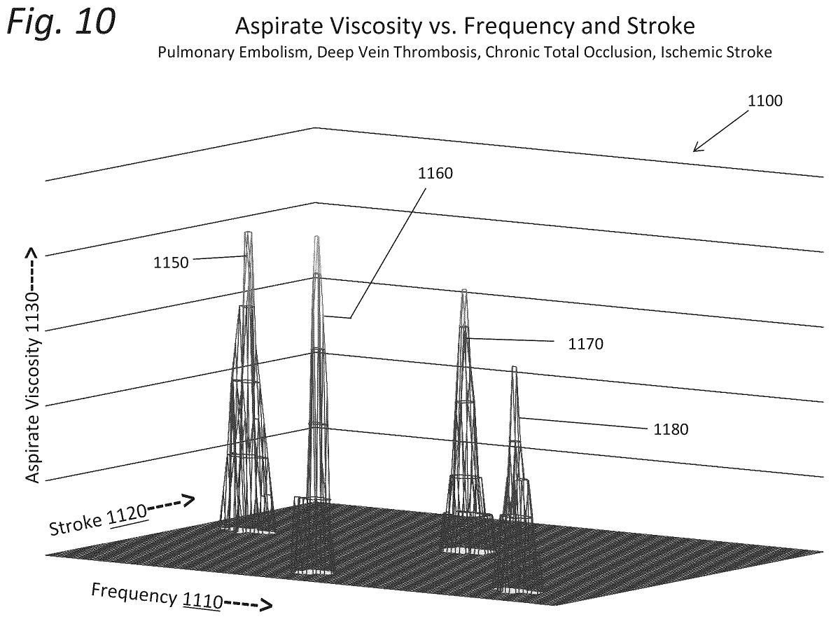

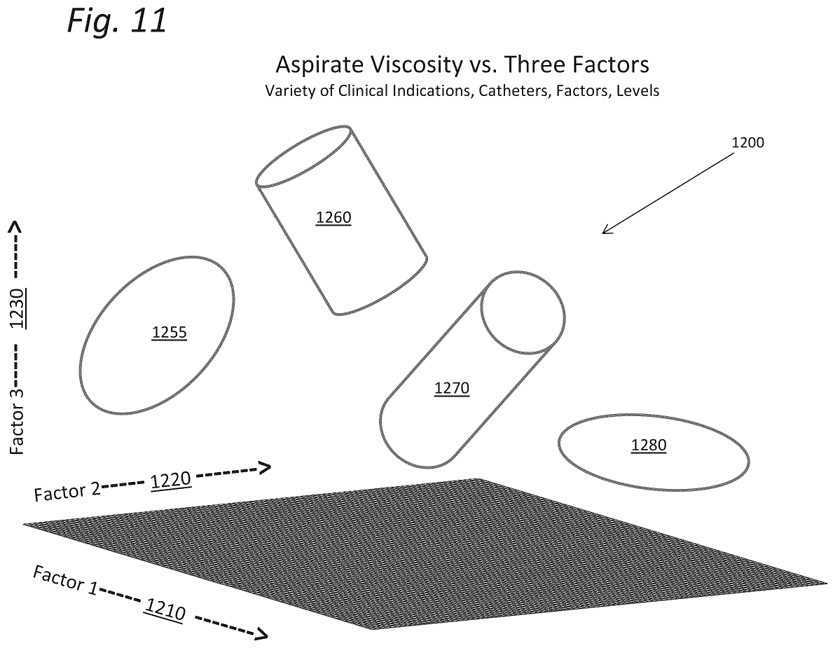

FIG. 6 depicted a representative graph of viscosity vs time as factor variables 782 and levels 788 are changed, however FIG. 6 does not present information about the magnitude or values of factor variables 782 and levels 788 that evoked each response. A speculation was provided based upon reasonable assumptions. However, other data analysis and graphical techniques enable a correlation between system settings (e.g., factor variables 782 and levels 788, etc.) and measured viscosity. FIG. 10 and FIG. 11 depict graphical two- and three-factor response surfaces as a plurality of factor variables 782 and levels 788 are explored. FIG. 10 and FIG. 11 are included for illustrative purposes, there is generally no need to contemplate generating such graphs. Furthermore, as the number of factor variable 782 exceeds three, graphical depiction is not generally possible.

FIG. 10 depicts a representative graphical example of a two-factor response surface 1100 where the first factor is frequency 1110 and the second factor is amplitude or stroke 1120; the ordinate (i.e., vertical axis) is aspirate viscosity 1130. Embodiments of the present invention include a broad array of catheter lengths, diameters and features (e.g., infusion tube 320, extension length 380, obturator, macerator, rotating cutter, etc.) to meet the clinical needs of a variety of thrombus types in a variety of anatomical locations. Example clinical indications include: pulmonary embolism 1150, deep vein thrombosis 1160, chronic total occlusion 1170, ischemic stroke 1180, etc. Two factor response surface 1100 depicts an increase in aspirate viscosity 1130 at four regions of the frequency-stroke plane or “space.” At low frequency 1110 and medium stroke 1120, a peak in aspirate viscosity 1130 is annotated pulmonary embolism 1150; this may be consistent with an inference from eq. 11 which predicts a low frequency response of a thrombus of generally larger mass. At higher frequency 1110 and lower stroke 1120, a peak in aspirate viscosity 1130 is annotated deep vein thrombosis 1160; again this may be consistent with eq. 11 which predicts a generally intermediate frequency response of a thrombus of generally intermediate mass. At higher frequency 1110 and medium stroke 1120, a peak in aspirate viscosity 1130 is annotated chronic total occlusion 1170; this may be consistent with eq. 11 which predicts a high frequency response of a thrombus of generally smaller mass. At higher frequency 1110 and short stroke 1120, a peak in aspirate viscosity 1130 is annotated ischemic stroke 1180; this may be consistent with eq. 11 which predicts a high frequency response of a thrombus of generally smaller mass; furthermore, the short stroke of the peak identified as ischemic stroke 1180 is anticipated because of the delicate nature of the surrounding vasculature. The graphical data presented in FIG. 10 is speculative; however it is based upon clinical indications and the governing equations of motion. Differences between appropriately-selected catheters (e.g., length, diameter, features, etc.) also influence the locations of each peak; a larger diameter catheter may generally tolerate a longer stroke because of the relationships outlined in Eq. 1 through Eq. 8. Historical data, including the location and expected value of each peak may be employed to minimize procedure times as the entire frequency—stroke plane or “space” need not be explored given the clinical indications and selected catheter. This foreknowledge of efficacious factors 780, levels 788, initial values, ranges 786, etc. may be supplied by means such as analysis of similar, previously-conducted thrombectomy procedures (historical data).

FIG. 11 depicts a representative example of a more general three factor response surface 1200 where generalized factors (e.g., factor(1) 1210, factor (2) 1220 and factor (3) 1230, etc.) are varied throughout the space to locate the (generally closed) response surfaces which are shown to bound a response volume. FIG. 11 depicts that, at low levels of factor (1) 1210 and factor (2) 1220 combined with intermediate levels of factor (3) 1230, type 1 thrombus 1255 appears as an oval, oblate spheroid or surface of revolution. The location, orientation, size and shape of oval representing type 1 thrombus 1255 may be determined by measuring aspirate viscosity 1130 or other means of determining the concentration of thrombus in the aspirate (e.g., flowmeter, optical density, etc.). Type 2 thrombus 1260 (response surface/volume) is illustrated at moderate levels of factor (1) 1210 and factor (2) 1220 and a higher level of factor (3) 1230. Type 3 thrombus 1270 is shown to be responsive to a range of levels of factor (1) 1210, factor (2) 1220 and factor (3) 1230. Type 4 thrombus 1280 is shown to be responsive to a limited range of levels of factor (2) 1220, a moderate range of levels of factor (3) 1230 and a broad range of levels of factor (1) 1210.

Heretofore in this disclosure, embodiments have been generally presented to execute “cause and effect” or “stimulus/response” methods that detect and exploit efficacious thrombectomy operating modes based upon intra-procedural data. The representative flowcharts of FIG. 9A through FIG. 9D evaluate viscometric measurement data with respect to other data including: baseline data (e.g., V₀, 4 cP, etc.), historical data, intra-procedural data, and/or arbitrary constants (e.g., δ₁, δ₂, κ, V_{clog}, etc.) etc. FIG. 7 depicts an example array of factors 780, factor variables 782 and levels 788 which had been predetermined by speculation or other undisclosed means. FIG. 7 is representative of a very large (and undetermined) number of combinations of the example factors 780 and levels 788; the number of combinations is certainly in excess of 1 million. It is therefore an objective of embodiments of the present invention to “pare down” the number of possible combinations (of factors and levels) to a manageable number, such as fewer than 50,000 possible combinations. It is a further objective of embodiments and methods of the present invention to utilize and contribute to a knowledge base of efficacious thrombectomy operating modes (e.g., in terms such as factors 780, factor variables 782 and levels 788) in order that initial values for levels 788, ranges 786 and number of levels 790 are selected that minimize procedure time and maximize thrombectomy efficacy.

For instance, a thrombectomy procedure to treat ischemic stroke will typically utilize a single-lumen catheter of small diameter (range of approximately 1F to 6F); therefore certain factors (e.g., infusion 895, extension 897, etc.) are typically not present in an ischemic stroke thrombectomy procedure. Likewise, in a typical ischemic stroke procedure, factor variable 782, LFO stroke 880 is typically limited to small values (e.g., 0.1 mm to 1.0 mm, etc.). This knowledge of clinical indication and catheter selection enable the selection of a predetermined set of arbitrary constants such as threshold values (e.g., δ₁, δ₂, κ, V_{clog}, etc.) and/or initial values such as loop indices (e.g., i=7, j=2, k=9, l=4, m=17, n=0, etc.) that are specific to the clinical indication and catheter.

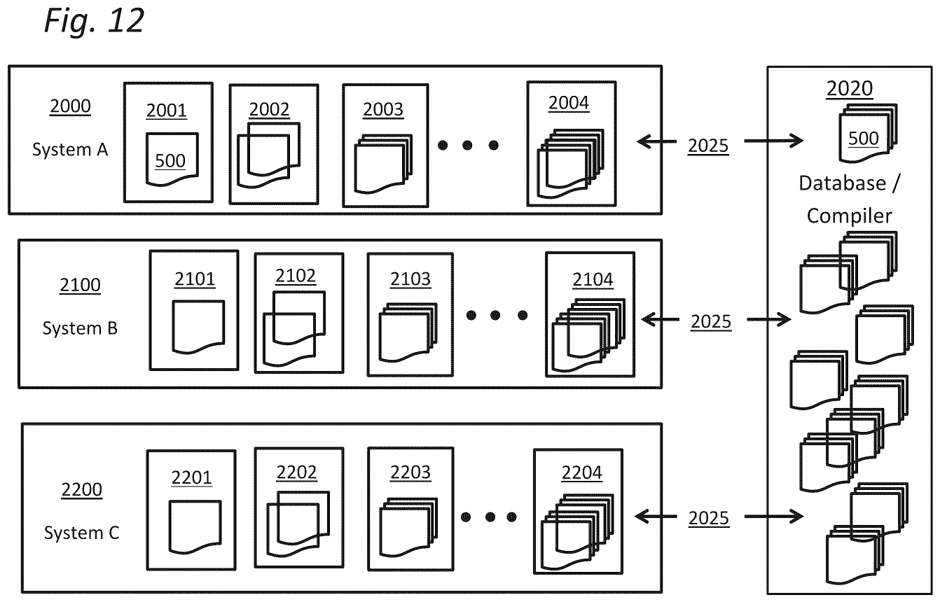

FIG. 12 illustrates a representative data storage, analysis and communication pathway suitable for determining and implementing new or updated system parameters (e.g., thrombectomy control flowcharts 901, threshold values and initial values for loop indices, etc.) that are specific to a clinical indication and catheter, etc., and derived from inter-procedural or historical data. A block diagram of an embodiment of a knowledge-based thrombectomy system is presented in FIG. 12 which depicts procedure data from multiple procedures being logged, transmitted to and compiled by database/compiler 2020. System A 2000, system B 2100, and system C 2200, are illustrated wherein a system may comprise any or all of the following: thrombectomy machine (by model number, serial number, location, etc.), catheter (model number, length, diameter, features, e.g., hydrodynamic jet, variable geometry, rotating cutter, obturator, etc.), system factors/parameters (e.g., LFO/HO, variable geometry, hydrodynamic jet, etc.), clinical indication (e.g., ischemic stroke, deep vein thrombosis, chronic total occlusion, pulmonary embolism, etc.), operator, etc. System A 2000 is shown to sequentially perform a first procedure denoted as system A procedure #1 2001; and subsequent procedures denoted as system A procedure #2 2002, system A procedure #3 2003 and system A procedure #n 2004. System A procedure #1 2001 is depicted to contain procedure log file 500; each procedure #x similarly contains procedure log file 500, however many instances are omitted in FIG. 12 for clarity. System B 2100 is shown to sequentially perform a first procedure denoted system B procedure #1 2101; and subsequent procedures denoted system B procedure #2 2102, system B procedure #3 2103 and B procedure #n 2104. System C 2200 is shown to sequentially perform a first procedure denoted system C procedure #1 2201; and subsequent procedures denoted system C procedure #2 2202, system C procedure #3 2203 and system C procedure #n 2204. Each procedure is shown to generate a procedure data log file, thus the n^{th} procedure is shown to generate an n^{th} procedure data log file for each procedure completed (for each system, e.g., A, B, C, etc.).

Database/Compiler 2020 is shown to exist remotely and is in shown in communication with system A 2000, system B 2100, and system C 2200 through data exchange 2025. Data exchange 2025 may comprise bi-directional wired or wireless communication or storage devices, e.g., Ethernet/internet, Bluetooth, WiFi, USB connection or storage devices, etc. Data collected from multiple thrombectomy procedures (e.g., as a plurality of procedure data log files, etc.) may be compiled and analyzed by database/compiler 2020 to identify effective treatment regimens (e.g., thrombectomy operating modes, initial values, arbitrary values, sequences or combinations thereof, etc.) that may significantly improve procedure efficacy. Database/compiler 2020 may then provide (data-driven) software or firmware updates to systems of the network (e.g., System A 2000, system B 2100, and system C 2200, etc.). Embodiments of the present invention integrate real-time measurement of thrombectomy efficacy (e.g., viscosity, relative flow rate, % thrombus, etc.) with correlation to a plurality of therapeutic treatments (thrombectomy operating modes) to enable cause/effect or stimulus/response analysis to be conducted both intra-procedurally and post-procedurally. FIG. 12 illustrates how inter-procedural data may be utilized in embodiments of the present invention because, for instance, a plurality of procedures conducted upon system A 2000 may precede subsequent similar procedure conducted upon system B 2100 and system C 2200, etc. A plurality of data log files 500 may be locally compiled and analyzed by individual systems (e.g., system A 2000, system B 2100, system C 2200, etc.) or by database/compiler 2020.

Embodiment of database/compiler 2020 is depicted, among other functionalities, as a repository for a plurality of procedure log files 500, each of which represents a statistically significant number of experiments (i.e., thrombectomy operating modes, range of approximately 100 to 20,000 per procedure) correlated to thrombectomy efficacy (e.g., viscosity, slope, % thrombus, relative flow rate, etc.). FIG. 12 is instrumental in conveying the quantity of data comprising embodiments of knowledge-based thrombectomy systems. Subsequent to any single procedure, clinicians, reviewers, analysts, algorithms or software may identify efficacious thrombectomy operating modes as well as inefficacious modes. These data are available to database/compiler 2020 for analysis including statistical analysis and development of robust thrombectomy control algorithms, initial values and arbitrary constants for system parameters such as: factors 780, factor variables 782, ranges 786, levels 790 and number of levels 790, etc. Updated software/firmware comprising knowledge-based thrombectomy control flowcharts 901 may be transmitted to and installed in individual thrombectomy systems (e.g., system A 2000, system B 2100, system C 2200, etc.) by data transfer means such as data exchange 2025, flash drives, CD-ROM, DVD-ROM, cloud storage, etc.

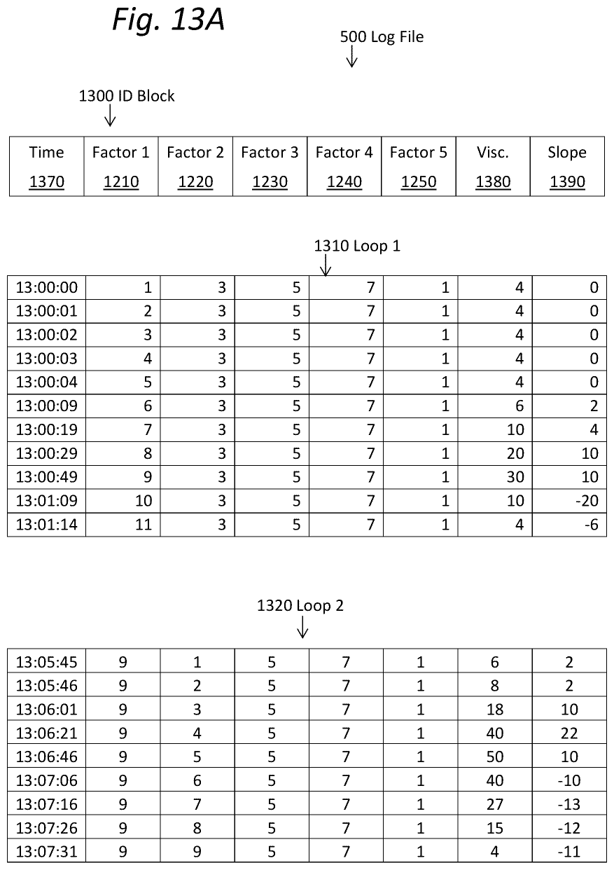

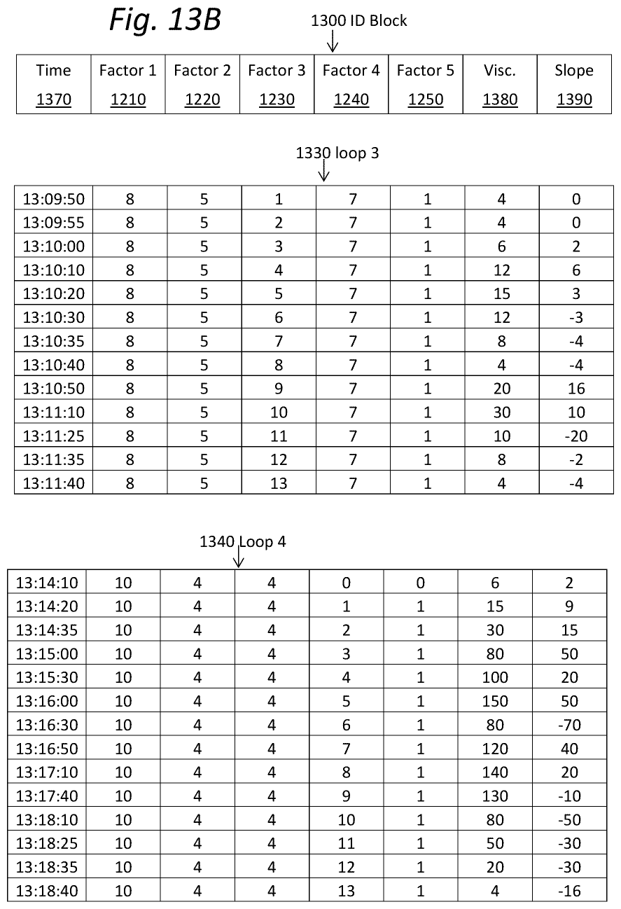

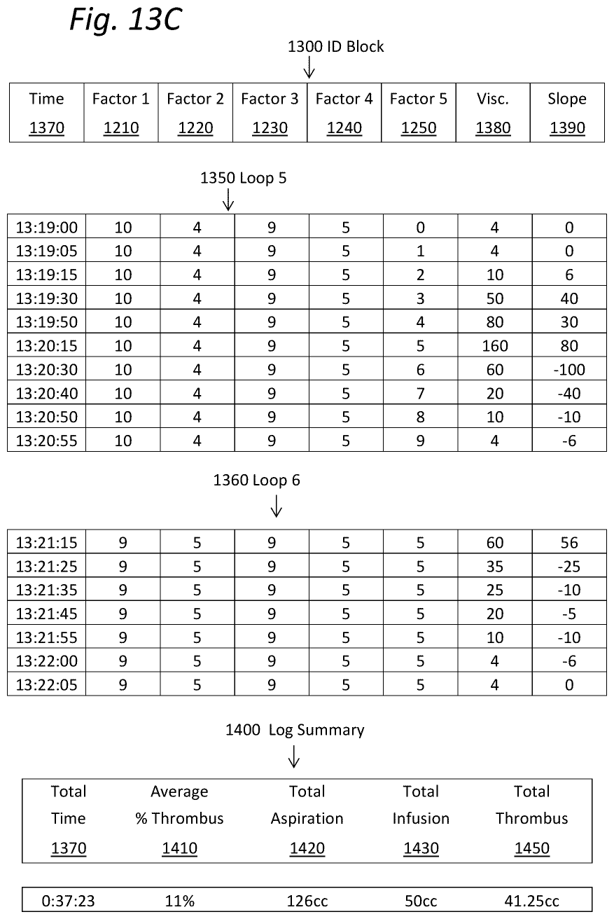

FIG. 13A, FIG. 13B and FIG. 13C show excerpts of a representative thrombectomy procedure data log file 500. This example thrombectomy procedure log file illustrates many of the functions of thrombectomy control flowchart 901, including adaptive dwell times, identifying efficacious combinations of i and j (loop indices) that positively correlate to thrombus in the catheter, updating loop indices to new values, etc. This example thrombectomy procedure log file illustrates an example progression or sequence of factors and levels consistent with a loop thrombectomy control flowchart 901 such as depicted in FIG. 9A; loops may be single, multiple or nested. ID block 1300 shows column headers including: time 1370, factor 1 1210, factor 2 1220, factor 3 1230, factor 4 1240, factor 5 1250, viscosity 1380 and slope 1390. Factors may be enumerated or a word description (e.g., 1, 2, 3, 4, etc. or infusion pressure, aspiration rate, LFO, HO frequency, etc.) may be shown (e.g., as headers) in a thrombectomy procedure log file. Thrombectomy procedure log file, including ID block 1300, may comprise data including, but not limited to: patient data, clinical indication, thrombectomy system, catheter, facility, clinician, software/firmware version, etc.

FIG. 13A depicts representative loop 1 1310 commencing at 13:00:00 hours and the initial time increment is shown to be 1 second; some data are omitted for brevity. Factor 1 1210 is initialized to level 1, factor 2 1220 is initialized to level 3, factor 3 1230 is initialized to level 5, factor 4 1240 is initialized to level 7, factor 5 1250 is initialized to level 1. The initial value levels may be predetermined (e.g., by knowledge-base, software/firmware, clinician input, etc.) or assigned arbitrary values; initial values may reflect historical data such that established, therapeutically efficacious, settings are selected. Loop 1 increments factor 1 1210 while the remaining factors and levels are held constant in this loop. Factor 1 1210 is shown to be incremented from 1 to 11 and an increase in aspirate viscosity is detected at level 6; the maximum measured viscosity (viscosity≈30) in loop 1 1310 is shown to occur at level 9 of factor 1 1210. Viscosity 1380 decreases to the nominal value for blood (approximately 4 cP) as factor 1 1210 is incremented to 11; loop 1 1310 is shown to be terminated after the experiment at level 11 returns a measured viscosity of blood (viscosity≈4). Level 9 of factor 1 1210 may be stored in memory as an efficacious level; in subsequent example loops, levels 8, 9 and 10 are shown to be upon.

Data from representative loop 1 1310 illustrate a feature of some embodiments of thrombectomy control flowchart 901, wherein efficacious thrombectomy operating modes are operated for a longer duration than less efficacious ones. For instance, at time 13:00:00, the measured aspirate viscosity and the duration of operation is approximately one second. At time 13:00:49, the measured aspirate viscosity is 30 cP, and the duration of operation is approximately 20 seconds. The representative data log files 500 (shown in FIG. 13A, FIG. 13B and FIG. 13C) illustrate how Effect Subroutine 1000 (among other capabilities) utilizes fixed and/or variable dwell times to extend the operating duration of efficacious thrombectomy operating modes (experiments).

Representative loop 2 1320 commences at 13:05:45 and holds factor 1 1210 level constant at the previously determined value of 9. Loop 2 1320 increments factor 2 1220, between 1 and 9, holding the remaining factors and levels constant. In loop 2 1320, the maximum measured viscosity (viscosity=50) is shown to occur at level 5 and returns to the measured viscosity of blood (viscosity≈4) at level 9 of factor 2 1220; loop 2 1320 is terminated when the measured viscosity decreases to that of blood (viscosity≈4). Level 5 of factor 2 1220 may be stored in memory as an efficacious level; in subsequent loops, levels 5 and 4 are experimented upon.

In FIG. 13B, loop 3 1330 commences at 13:09:50. Factor 3 1230 is incremented between 1 and 13 in loop 3 1330 while factor 1 1210 is held constant at level 8 (determined during loop 1 1310) and factor 2 1220 is held constant at level 5 (determined during loop 2 1320). The remaining factors and levels remain at initial values. Loop 3 1330 is shown to have two viscosity peaks occurring at level 5 (viscosity=15) and level 10 (viscosity=30); loop 3 1330 is terminated at level 13 of factor 3 1230. Levels of 5 and 10 of factor 3 1230 may be stored in memory as efficacious levels; in subsequent loops levels 4 and 9 of factor 3 1230 are explored. Deviations from the efficacious levels (e.g., 5; 10, etc.) are subsequently experimented upon (e.g., 4 and 6 as deviations from 5; 8, 9, 11 and 12 as deviations from 10, etc.).

Loop 4 1340 commences at 13:14:10; factor 4 1240 is incremented between levels 0 and 13. In loop 4 1340, factor 1 1210 is held constant at level 10, factor 2 1220 and factor 3 1230 are held constant at level 4; these levels (or deviations therefrom) had been determined during previous loops. Loop 4 1340 is shown to have two viscosity peaks occurring at level 5 (viscosity=150) and level 8 (viscosity=140). Levels of 5 and 8 of factor 3 1230 may be stored in memory as efficacious levels; in subsequent loops, only level 5 of factor 3 1230 is shown to be explored (for brevity).

FIG. 13C shows loop 5 1350 commencing at 13:19:00; factor 5 1250 is incremented between levels 0 and 9. In loop 5 1350, factor 1 1210 is held constant at level 10, factor 2 1220 is held constant at level 4, factor 3 1230 is held constant at level 9 and factor 4 1240 is held constant at level 5. In loop 5 1350, four of the five factors have levels that were determined in previous loops. Loop 5 1350 is shown to have a viscosity peak occurring at level 5 (viscosity=160). Level 5 of factor 4 1240 may be stored in memory as an efficacious level; in loop 6 1360, only level 5 of factor 5 1250 is shown to be explored (for brevity).

Loop 6 1360 commences at 13:21:15. No factors are incremented in loop 6 1360; all factors and levels were determined in previous loops. Loop 6 1360 illustrates that all factors and levels may be held constant (experiments are repeated) while the measured viscosity decreases from 60 (at 13:21:15) to 4 (at 13:22:00).

Log summary 1400 depicts total procedure time 1370 (0:37:23), average % thrombus 1410 (11%), total aspiration 1420 (126cc), total infusion 1430 (50cc), and total thrombus 1450 (41.25cc). Log summary 1365 is a “procedure scorecard” of a thrombectomy procedure data log file; the relevant data may be averaged, statistically analyzed, compiled or otherwise manipulated and organized into relevant contributions to a knowledge base. A thrombectomy procedure featuring: minimum procedure time 1370, coupled with a maximum average % thrombus 1410, minimum total aspiration 1420 (blood loss) and maximum total thrombus 1450 is desirable.

Thrombectomy procedure log file 500 (as illustrated in FIG. 13A, FIG. 13B and FIG. 13C) is shown to contain measurement data (viscosity and slope) that were used as arguments of conditional statements (e.g., IF-THEN, DO—WHILE, etc.) within a thrombectomy control flowchart 901. In this manner, data are utilized intra-procedurally to identify and select combinations of factors 780 and levels 788 for extended operation (e.g., by means such as dwell commands, repeating combinations, etc.) or for further experimentation. Thrombectomy procedure log file 500 also illustrates how efficacious combinations of factors 780 and levels 788 may be stored as predetermined values for subsequent loops and/or procedures. Subsequent procedures sharing common clinical indications and equipment (e.g., PE with catheter XYZ, DVT with catheter ABC, etc.) may benefit from any (or all) prior thrombectomy procedure data log files to provide predetermined thrombectomy system settings including: initial values, ranges, dwell times, threshold values, selected factors and ranges, etc.

Procedure log file 500 illustrates how example thrombectomy control algorithm 901 responds to the data and correlation in two ways: (1) the dwell time is increased for efficacious configurations, and (2) efficacious configurations/setpoints (e.g., Factor 1 1210 set to level 788=10, Factor 2 1220 set to level 788=4, etc.) are repeated a plurality of times throughout the remainder of the procedure. Thrombectomy control algorithm 901 prescribes a sequence of operations of a thrombectomy apparatus; each operation of the apparatus occurring for a duration sufficient to take a measurement of thrombus in the catheter. Upon detection of an operation that positively correlates to thrombus, thrombectomy control algorithm 901 invokes actions (e.g., DWELL, j=j-1, etc.) that interrupt or change the prescribed sequence. Embodiments of the invention thereby implement control strategies that are adaptive to increase the procedure time wherein flowing thrombus is present in the catheter.

Procedure log file 500 illustrates a difference between intra-procedural and inter-procedural (post-procedural or historical) data as utilized by some embodiments of the invention. The example procedure utilized initial values (for apparatus or system setpoints) of (1, 3, 5, 7, 1) and a set of arbitrary constants, levels, ranges and threshold values (e.g., δ₁, δ₂, κ, etc.). During the course of the procedure, it was determined that setpoint configuration (10, 4, 9, 5, 5) exhibited strong correlation to viscosity. Had the procedure utilized the initial values (10, 4, 9, 5, 5), the procedure efficiency may have been increased. Embodiments of the invention compile a plurality of similar procedure log files 500 and calculate improved initial values; improved values for levels, ranges and threshold values may be calculated or otherwise obtained by the historical data. The improved set of initial values, levels, ranges and threshold values may be transmitted to each system controller 810 such as by software or firmware update. Thrombectomy control algorithm 901 may be improved by observation or analysis of historical data such that a more efficient set of machine instructions may be transmitted to each system controller 810 such as by software or firmware update.

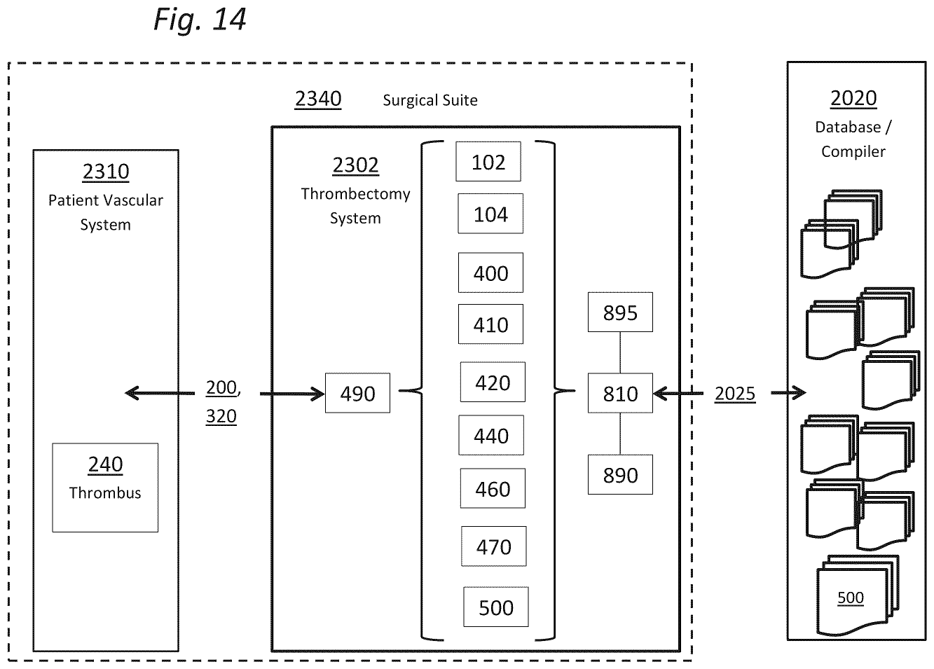

FIG. 14 depicts a physical relationship and communication pathways representative of some embodiments of a generalized knowledge-based thrombectomy system 2302. FIG. 14 depicts a block diagram of a knowledge-based thrombectomy system 2302 with respect to the patient and patient vascular system 2310, surgical suite 2340 (the location where the thrombectomy procedure is performed) and remote database/compiler 2020, that may typically be at an off-site and/or centralized location. Catheter 200 and infusion tube 320 are shown to access patient vascular system 2310 which is shown to contain thrombus 240.

Knowledge based thrombectomy system 2302 is shown comprising any or all of system controller 810, LF Oscillator 102, Harmonic Oscillator 104, LCO with aspiration and infusion 400, accelerometer 410, pressure transducer 420, aspirate pump 440, infusion pump 460, infusion tube drive motor 470, and manifold 500; other embodiments may comprise a greater or lesser number of components or features which may be implemented to aspirate thrombus 240. System controller 810 is shown in communication with clinician input 895 and clinician data 890; communication with database/compiler 2020 is shown through data exchange 2025.

FIG. 15 depicts a block diagram of an illustrative system controller 810 operating in accordance with aspects and implementations of the present disclosure. As shown in FIG. 15, system controller 810 comprises processor 2402, main memory 2404, storage device 2406, analog/digital I/O 2408, motor drivers 2410, audio generator 2412, video generator 2414, interconnected as shown (e.g., via one or more busses, etc.). System controller 810 may comprise proprietary hardware or off-the-shelf components including PC's, microcontrollers (e.g., Arduino, Raspberry Pi, etc.) that may be programmed in any appropriate computer language including Visual Basic, C++, Python, etc. Syntax from BASIC and FORTRAN computer programming languages are incorporated herein including: FOR—NEXT loops, IF-THEN conditional statements, DO loops, DO WHILE loops, GOSUB, GOTO, etc.

Processor 2402 represents one or more general-purpose processing devices such as a microprocessor, central processing unit, or the like. More particularly, processor 2402 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. Processor 2402 may also be one or more special-purpose processing devices such as an application specific integrated circuit (ASIC), a field programmable gate array (FPGA), a digital signal processor (DSP), network processor, or the like. Processor 2402 is capable of executing instructions stored in main memory 2404 and storage device 2406, including instructions corresponding to the methods exemplified in FIG. 9A through FIG. 9D; of reading data from and writing data into main memory 2404 and storage device 2406; and of receiving input signals and transmitting output signals to analog/digital I/O 2408. While a single processor is depicted in FIG. 15 for simplicity, system controller 810 might comprise a plurality of processors.

Main memory 2404 is capable of storing executable instructions and data, including instructions and data corresponding to the methods exemplified in FIG. 9A through FIG. 9D, and may include volatile memory devices (e.g., random access memory [RAM]), non-volatile memory devices (e.g., flash memory), and/or other types of memory devices. Software /firmware 2416 may comprise a list of executable instructions and data and may be integrated to main memory 2404 or as a standalone, discrete component.

Storage device 2406 is capable of persistent storage of executable instructions and data, including instructions and data corresponding to the method exemplified in FIG. 9A through 9D, and may include a magnetic hard disk, a Universal Serial Bus [USB] solid state drive, a Redundant Array of Independent Disks [RAID] system, a network attached storage [NAS] array, etc. While a single storage device is depicted in FIG. 15 for simplicity, system controller 810 might comprise a plurality of storage devices.

Analog/Digital I/O 2408 receives input signals from one or more devices including pressure transducer 420, accelerometer 410 and/or a user of system controller 810. Analog/Digital I/O 2408 forwards corresponding signals to processor 2402, receives signals from processor 2402, and emits corresponding output signals that can control system functions (e.g., aspirate pump 440, infusion pump 460, infusion tube drive motor 470, LCO 100, etc.) and/or be sensed by the user. The input mechanism of analog/digital I/O 2408 might be a footswitch, a knob, an alphanumeric input device (e.g., a keyboard, etc.), a touchscreen, a cursor control device (e.g., a mouse, a trackball, etc.), a microphone, etc., and the output mechanism of analog/digital I/O 2408 might be a liquid-crystal display (LCD), a cathode ray tube (CRT), a speaker, etc. While a single I/O device is depicted in FIG. 15 for simplicity, system controller 2402 might comprise a plurality of I/O devices.

Audio generator 2412 and/or video generator 2414 may produce information, data or signals which are conveyed to the clinician by means including a speaker and/or a touchscreen, and/or a LCD and/or CRT display. Motor drivers 2410 may produce signals which provide control over electric devices such as motors, step motors, servo motors, linear actuators, solenoids, valves etc. Database/compiler 2020 is shown in communication with system controller 810 by communication means such as data exchange 2025. Data exchange 2025 may provide unidirectional or bi-directional communication with system controller 810 by which information including data log file 500, and/or software/firmware 2416 updates are transmitted. Data exchange 2025 may comprise wired connection (e.g., USB port, DB-9, DB-25 connections, etc.) or may comprise wireless communication (e.g., Bluetooth, WiFi, cloud storage or other wireless network communication protocol.)

It is to be understood that the above-described embodiments are merely illustrative of the present invention and that many variations of the above-described embodiments can be devised by those skilled in the art without departing from the scope of the invention. For example, in this specification, numerous specific details are provided in order to provide a thorough description and understanding of the illustrative embodiments of the present invention. Those skilled in the art will recognize, however, that the invention can be practiced without one or more of those details, or with other methods, materials, components, etc.

Furthermore, in some instances, well-known structures, materials, or operations are not shown or described in detail to avoid obscuring aspects of the illustrative embodiments. It is understood that the various embodiments shown in the figures are illustrative, and are not necessarily drawn or labeled to scale. Reference throughout the specification to “one embodiment” or “an embodiment” or “some embodiments” means that a particular feature, structure, material, or characteristic described in connection with the embodiment(s) is included in at least one embodiment of the present invention, but not necessarily all embodiments. Consequently, the appearances of the phrase “in one embodiment,” “in an embodiment,” or “in some embodiments” in various places throughout the specification are not necessarily all referring to the same embodiment. Furthermore, the particular features, structures, materials, or characteristics can be combined in any suitable manner in one or more embodiments. It is therefore intended that such variations be included within the scope of the appended claims and their variants or equivalents.

This disclosure of the invention incorporates multiple embodiments including means to attrite thrombus, means to aspirate thrombus and means to measure thrombus; additionally, methods to conduct deterministic events (experiments) and correlate measurement data to apparatus configuration (e.g., setpoints, positions, etc.). The multiple embodiments and multiple methods disclosed herein gives rise to a large combinations thereof. A non-exhaustive list of combinations of embodiments and methods is provided herein for completeness. Some embodiments of the invention comprise (in addition to other structures, mechanisms or embodiments) any combination of: Liquid Column Oscillator (LCO), Low Frequency Oscillator, Harmonic Oscillator, impulse mechanism, pressure limiter, hydrodynamic lance, mechanical lance, manifold, valve, obturator, guidewire, rotating cutter, stent retriever, capture mesh, filter, variable catheter geometry, system controller, peristaltic pump, pressurized or evacuated reservoir, liquid pump, gas/vapor/vacuum pump, syringe, syringe pump, non-isovolumetric component, stepper motor, servo motor, linear actuator, pressure transducer, flowmeter, stent retriever, capture mesh, thrombectomy procedure data log files, visual display, audio speaker, clinician input, footpedal, knobs, keypad, manual or automated positioning of, and position measurement of devices including: catheters, guidewires, obturators, etc. Some embodiments of the invention utilize methods including any combination of: time-domain viscometry, viscometric sampling, viscometric distance sampling, determination of % thrombus, determination of thrombus load, determination of (relative and absolute) flow rate, determination of thrombectomy efficacy, clog detection, clog aversion, dynamic or adaptive modification of a sequence of machine instructions, data analysis, software or firmware updates, communication pathways, determination of threshold values, determination of ranges and increments for setpoint adjustment of an apparatus, pressure limitation, causing cavitation or boiling, determination and excitation of resonant frequencies, cause and effect (stimulus/response) experimentation, valve actuation, DOE, SPC, thrombectomy control algorithms, effect subroutines, distance subroutines, clog detect/avert subroutines, viscosity/slope measurement subroutines, thrombectomy procedure data log files, graphical depiction, clinician instructions, clinician feedback, clinician input, calibration procedure and subroutine, apparatus operation, setpoint changes, etc.

In order to provide a clear and consistent understanding of the disclosure and the appended claims, including the scope to be given such terms, the following glossary of terms and definitions is provided.

Liquid Column Oscillator (LCO): any combination of an oscillating or reciprocating surface fluidically coupled to a fluid-filled catheter operating in the range of approximately 0.1 Hz to 19,000 Hz. LCO embodiments may comprise a plurality of oscillating or reciprocating surfaces including: LF oscillator and Harmonic Oscillator (HO).

LF Oscillator (low frequency): A Liquid Column Oscillator characterized by imparting fluid translation within a catheter on a length scale of approximately 0.5 mm to 500 mm and a frequency range of approximately 0.1 Hz to 50 Hz. The abbreviation LFO may be used herein; the terms LCO, Low Frequency Oscillator and LFO may be used interchangeably in context herein.

HF Oscillator (High Frequency or Harmonic Oscillator): A Liquid Column Oscillator characterized by imparting pressure or mechanical waves within a fluid medium wherein the fluid translation is typically less than approximately 0.5 mm and a frequency range of approximately 20 Hz to 19,000 Hz. The terms LCO, HO, Harmonic Oscillator, HF Oscillator and High Frequency Oscillator may be used interchangeably in context herein.

Fluid: any homogeneous or heterogeneous matter comprised of vapors, gasses, liquids, solids, suspensions or slurries thereof that may be transferred into a tube, pipe or catheter by means of a differential pressure existent within the tube, pipe or catheter. The terms Fluid and Liquid may be used interchangeably herein.

Viscosity—the resistance of a fluid to flow; herein including the resistance of a homogeneous fluid or inhomogeneous mixture of fluids and/or solids to flow through a catheter or conduit. An inhomogeneous mixture of thrombus and blood might be uniformly distributed or might be non-uniform (spatially discrete) along the length of a catheter. The viscosity of an inhomogeneous mixture in a catheter may be measured by methods such as time-domain viscometry. The measured viscosity may be approximately inversely proportional to the rate of flow through the catheter. Herein, viscosity may be construed to mean the average or effective viscosity of fluid contained within a conduit or catheter. Eq. 0 provides a mathematical description where \hat{μ} is the viscosity of a differential fluid volume element; the effective or average viscosity may be calculated by integrating the viscosities of the differential fluid volume elements along the length (L) of the catheter.

$$µ \approx \frac{1}{L} {\int }_{0}^{L} \hat{µ} dx \qquad Eq . 0$$

Note that if even a single differential fluid volume element has a very large value of \hat{μ} (e.g., greater than approximately 10,000 cP) the measured, effective or average value of μ will also increase to a large value. Thus, a clogged or corked catheter might be indicated by a very large measured value of μ.

Viscometric Sampling—A method of measuring the viscosity of aspirate—or—a method of determining an aspirate characteristic, wherein a small aspirate sample (range of approximately 0.2cc to 5.0cc) is withdrawn from a patient vasculature to quantitatively measure the viscosity. In cases wherein the measured aspirate viscosity is approximately equal to blood, the sample may be returned to the patient vasculature, such as by reversal of an aspirate pump. Viscometric sampling may thereby be conducted with small, zero or near-zero blood loss. Viscometric sampling data in excess of threshold values (e.g., 500 cP, 1,000 cP, etc. as may be experimentally determined) may detect a clog, imminent clog or corking of the catheter.

Viscometric Distance Sampling—A method of viscometric sampling that includes an inference to the distance between the catheter tip and a thrombus. As the catheter tip is physically moved (i.e., advanced) into closer proximity (i.e., smaller distance) to a thrombus, the magnitude of data from viscometric sampling increases in inverse relation to distance. Viscometric distance sampling data in excess of threshold values (e.g., 500 cP, 1,000 cP, etc. as may be experimentally determined) may detect a clog, imminent clog or corking of the catheter.

Corking (of a Catheter)—A case of clogging a catheter wherein an excess of thrombus is ingested such that ordinary differential pressure (e.g., steady, interrupted, non-oscillatory, etc. suction/vacuum from a syringe, evacuated reservoir, etc.) forces are ineffective in establishing or restoring flow. Corking of a catheter typically occurs when suction /vacuum exceeds maximum levels imposed by constraints including mass, diameter, length, rheology, viscosity, solids content, rigidity, elasticity, etc. of thrombus with respect to the catheter dimensions.

Aspirate (noun)—any fluid, liquid, solid, slurry, homogeneous or heterogeneous matter that may be transferred through a conduit or catheter; also the contents of the conduit or catheter.

Aspirate or aspirating (verb)—employ(ing) differential pressure to transfer any fluid, liquid, solid, gas, vapor, slurry or heterogeneous matter through a conduit or catheter. Sources of differential pressure include: pumps, evacuated reservoirs, syringes, compliance chambers, atmospheric pressure, intravascular pressure, phase change, gravity, etc.

Attribute Data: Attribute data, also known as categorical data, consists of discreet categories or labels that represent different qualities or characteristics. These categories are typically non-numeric and qualitative in nature. Attribute data is typically used to describe characteristics that can be counted or categorized. Attribute data are typically graphically depicted as frequency distributions, histograms, bar charts, pie charts, etc.

Variable Data: Variable data, also known as numerical data or continuous data, consists of numeric values that can take any real number value within a certain range. These values can be measurements of physical properties and are quantitative in nature. Variable data may be expressed in engineering units including: cP, Pa, ° C., etc.; variable data may be ratiometric or expressed as percentages. Variable data may be acted upon using mathematical methods of algebra, linear algebra, calculus, differential equations, etc.

Experiment: herein, the act of exposing a system to external stimuli and measuring the system response. Example: a thrombectomy apparatus is operated wherein the setpoint(s) of the apparatus have been adjusted to specific values (e.g., aspiration pump speed=10%, infusion pump speed=20%, LCO frequency=15 Hz, etc.); this operation is maintained for a duration enabling measurement of the system response (e.g., viscosity=10 cP, % thrombus=10%, relative flow rate=40%, etc.). Also, an investigation and attempt to demonstrate the cause and effect relationship between two or more variables (factors and/or levels).

Experiment array: herein, a shorthand notation that indicates the setpoint(s) of the apparatus during an experiment. Example experiment arrays: [aspiration pump speed=10%, infusion pump speed=20%, LCO frequency=15 Hz, infusion=0%, rotating cutter=0 rpm, HO frequency=3,000 Hz] or simply [10, 20, 15, 0, 0, 3,000].

Correlation: herein, connecting or associating an experiment or experiment array with the measured system response (e.g., viscosity=10 cP, % thrombus=10%, relative flow rate=40%, etc.). The correlation may be termed “positive correlation” when the measured system response is measured to be outside of baseline values (e.g., viscosity>4 cP, % thrombus>0%, relative flow rate<100%, etc.). Positive correlation may be quantitative. Example: experiment X [10, 20, 15, 0, 0, 3,000] is correlated to a measured % thrombus of 30%. Experiment X is positively correlated to % thrombus at 30% significance, or [10, 20, 15, 0, 0, 3,000] is positively correlated to a measured % thrombus at 30% significance.

Deterministic event: herein, any prescribed event that affects, influences or changes the outcome of successive events, observations or measurements. Herein, conducted experiments are deterministic events that affect, influence or change the outcome of thrombus attrition or thrombectomy efficacy measurements.

Design of Experiments (DOE): a systematic, efficient method that enables study of the relationship between multiple input variables (i.e., factors), at multiple input setpoints (i.e., levels), and key output variables (i.e., responses, e.g., thrombectomy efficacy). It is a structured approach for collecting and analyzing data.

Thrombectomy Operating Mode: Any combination of factors (e.g., aspiration, infusion, LCO/HO, rotating cutter, obturator, hydrodynamic jet, variable geometry, catheter positioning, etc.) and levels (e.g., on/off, 375 Hz, 10% speed, 0.8cc/s flow rate, 40% pressure, direct impingement, 10% extension, 5 mm withdrawal, etc.) which may be executed in the course of a thrombectomy procedure.

Thrombectomy Control Flowchart (or Thrombectomy Control Algorithm): An algorithm to execute a plurality of thrombectomy operating modes and measure the thrombectomy efficacy of each thrombectomy operating mode. A thrombectomy control flowchart algorithm may invoke intra-procedural data, clinician input, predetermined values, historical data and/or conditional statements to improve the efficacy of a thrombectomy procedure by identifying and exploiting efficacious thrombectomy operating modes.

Thrombectomy and/or Procedure Data Log File: a generally tabular, matrix or graphical form of collected data comprising thrombectomy system settings (e.g., factors and levels, parameters and setpoints, thrombectomy operating mode, etc.) correlated to measured thrombectomy efficacy.

Thrombectomy Efficacy: (1) An intra-procedural quantitative score system to assess the amount of thrombus and the flow rate within the catheter at any point in time. Thrombectomy efficacy may be defined to be zero at any time if either the % thrombus in the catheter is zero or if the flow rate is zero (e.g., due to a clog). (2) A post-procedural quantitative score system to assess measured quantities such as the amounts of thrombus, blood loss and procedure time.

Factor: an adjustable apparatus, machine or system parameter (e.g., speed, frequency, pressure, length, temperature, etc.); factors are typically setpoint controlled.

Level: a setpoint of a factor (e.g., speed setpoint=10 mph, frequency setpoint=375 Hz, pressure setpoint=50 psi, length setpoint=10 mm, temperature setpoint=298K, etc.). In some cases (e.g., involving manual manipulation), the level may be measured (e.g., catheter position, obturator position, etc.).

Setpoint: (1) automated systems: the desired value of any adjustable process variable (e.g., pressure, flow rate, rpm, frequency, length, etc.). (2) manually operated systems: a stated or measured value of any adjustable process variable (e.g., catheter position, footpedal position, knob adjustment, regulator adjustment, etc.).

Characteristic of Flow: A language-based descriptor of flow of aspirate within a catheter which is expressed in attribute data terms including: “free flow,” “open flow,” “restricted flow,” “clot,” “clog,” etc. Also termed “Flow State.” Characteristic of flow may be monitored for change.

Relative Flow Rate (Qr): An intensive property of fluid within a catheter calculated from viscosity measurements. Relative flow rate may be normalized to a reference fluid such as blood or saline. Relative flow rate is generally independent of scale (e.g., catheter length/diameter, differential pressure, etc.). Qr (calculated for the i^{th} sample) may be defined in a manner such as: Qrᵢ=(μ_{blood})/(μᵢ) (where Qrᵢ is the relative flow rate of the i^{th} sample and μᵢ is the viscosity of the i^{th} sample) or a similar expression.

Absolute Flow Rate (Q) and Absolute Flow Velocity: extensive properties of a flow field that may be quantitatively expressed in units such as liters per minute (I/min) or meters per second (m/s). Absolute flow rate and absolute flow velocity are generally dependent upon differential pressure, catheter dimensions, etc. Absolute flow rate and absolute flow velocity may be interconverted multiplying/dividing by the diameter of the catheter, tube or pipe; this conversion may be approximate because of phenomena including fluid velocity profile.

% Thrombus or % T: An intensive property of aspirate (within a catheter) derived or calculated from viscosity measurements. % Thrombus is relevant in thrombectomy vernacular because: (1) larger is better, (2) the measurement data has been converted to intuitively familiar units. % Thrombus (calculated for the i^{th} sample) may be defined in a manner such as % Tᵢ=(1−Qrᵢ)×100% (where % Tᵢ is the calculated % thrombus for the i^{th} sample) or a similar expression.

Thrombus Load: a quantitative property of aspirate (within a catheter) derived or calculated from viscosity measurements in conjunction with historical data that identify threshold viscosities that, if exceeded, are likely to cause clogging or corking of a catheter. For example, in cases wherein thrombus load is calculated to be 30%, maximum aspiration may be continued (perhaps increasing the thrombus load). In cases wherein thrombus load is calculated to be 90%, actions such as: reduced aspiration rates, catheter repositioning, saline flush, etc. are indicated to avert imminent clogging or corking of the catheter.

Means to Attrite Thrombus: herein, any apparatus, structure, mechanism or system to attrite thrombus by mechanical, hydrodynamic, pressure or oscillatory forces or actions. Examples include: Liquid Column Oscillator, Low Frequency Oscillator, Harmonic Oscillator, obturator, guidewire, hydrodynamic lance, hydrodynamic jet, rotating cutter, snare, stent retriever, catheter, aspiration system, thrombolytic compounds, etc. Example setpoints include: 3 Hz, 10 RPM, 3 mm, 30%, 10 mmHg, 100 psi, 3 seconds, 3 revolutions, on/off, open/closed, deployed, etc.

Means to Aspirate Fluid: herein, any apparatus or mechanism, such as a pump (liquid, gas or vapor) or pumping system, valve, differential pressure reservoir, etc., that causes flow of fluid through a catheter. A means to aspirate fluid may also comprise a means to attrite thrombus. Examples include: peristaltic pump, positive displacement liquid pump, evacuated reservoir, vacuum pump, dynamic pump, hydrodynamic jet, valve, syringe, syringe pump, etc. Example setpoints include: 3 Hz, 10 RPM, 3 mm, 10 mmHg, 100 psi, 3 seconds, 3 revolutions, 30%, on/off, open/closed, etc.

Means to Measure Thrombus: herein, any instrument or system that directly or indirectly measures the amount of thrombus within a catheter, including measurements of a fluid's resistance to flow through a catheter. Examples: viscometer, time-domain viscometer, flowmeter, differential pressure flowmeter, pressure transducer, densitometer, thermistor, etc.

The following referencing numbers are used in this disclosure:

| | |
|---|---|
| 10 | Pressure Limiter |
| 15 | orifice plate |
| 17 | orifice |
| 20 | pressure limiter piston |
| 22 | exhaust port/tube |
| 25 | pressure limiter spring |
| 30 | pressure limiter cylinder |
| 35 | crankshaft rotation direction |
| 40 | piston direction |
| 45 | cylinder pressure |
| 50 | pressure limiter pressure |
| 55 | pressure limiter displacement |
| 60 | torsion spring |
| 65 | impulse piston |
| 70 | sear |
| 75 | hammer |
| 80 | impulse piston distance |
| 90 | impluse mechanism |
| 100 | Liquid Column Oscillator (LCO) |
| 102 | LF oscillator (LF) |
| 104 | harmonic oscillator (HO) |
| 110 | sonic transducer |
| 120 | piston |
| 130 | acoustic tube |
| 140 | connecting rod |
| 150 | slide |
| 160 | crankshaft |
| 170 | high frequency standing wave |
| 180 | motor |
| 190 | low frequency standing wave |
| 200 | cylinder |
| 220 | catheter |
| 225 | vascular access |
| 230 | catheter tip |
| 240 | thrombus |
| 245 | thrombus bolus |
| 250 | tip to thrombus distance |
| 260 | attachments |
| 280 | vessel wall |
| 290 | oscillatory flow |
| 294 | aspiration flow direction |
| 295 | aspirate flow |
| 296 | infusion flow direction |
| 298 | P1 |
| 299 | P2 |
| 300 | thrombus amplitude |
| 320 | infusion tube |
| 340 | axial nozzle |
| 360 | radial nozzle |
| 380 | extension |
| 400 | LCO with aspiration and infusion |
| 410 | accelerometer |
| 420 | pressure transducer |
| 440 | aspirate pump |
| 450 | waste tube |
| 460 | infusion pump |
| 470 | infusion tube drive motor |
| 475 | coiled infusion tube |
| 480 | supply tube |
| 490 | manifold |
| 500 | log file |
| 510 | FS1 |
| 520 | FS2 |
| 530 | FS3 |
| 560 | radial jet |
| 580 | axial jet |
| 710 | blood |
| 730 | phase 1 |
| 750 | phase 2 |
| 770 | phase 3 |
| 780 | factor |
| 782 | factor variable |
| 784 | units |
| 786 | range |
| 788 | levels |
| 790 | number of levels |
| 792 | loop termination |
| 810 | system controller |
| 860 | LFO Frequency |
| 865 | HO Frequency |
| 875 | acoustic length |
| 880 | LFO Stroke |
| 885 | aspiration rpm |
| 890 | clinician data |
| 895 | clinician input |
| 895 | infusion rpm |
| 897 | extension length |
| 901 | thrombectomy control flowchart |
| 902 | operate apparatus |
| 905 | start |
| 910 | initialize setpoints |
| 920 | position catheter |
| 930 | factor loop |
| 940 | level loop |
| 944 | advance catheter |
| 946 | TOM = f{V(i, j)} |
| 948 | retract catheter |
| 950 | measure viscosity and slope |
| 960 | GOSUB 1000 |
| 970 | next level |
| 980 | next factor |
| 1000 | effect subroutine 1000 |
| 1050 | distance subroutine 1050 |
| 1060 | clog detect/avert subroutine 1060 |
| 1070 | viscosity/slope subroutine 1070 |
| 1100 | two-factor response surface |
| 1110 | frequency |
| 1120 | stroke |
| 1130 | aspirate viscosity |
| 1150 | pulmonary embolism |
| 1160 | deep vein thrombosis |
| 1170 | chronic total occlusion |
| 1180 | ischemic stroke |
| 1200 | three factor response surface |
| 1210 | factor 1 |
| 1220 | factor 2 |
| 1230 | factor 3 |
| 1240 | factor 4 |
| 1250 | factor 5 |
| 1255 | type 1 thrombus |
| 1260 | type 2 thrombus |
| 1270 | type 3 thrombus |
| 1280 | type 4 thrombus |
| 1300 | title block |
| 1310 | loop 1 |
| 1320 | loop 2 |
| 1330 | loop 3 |
| 1340 | loop 4 |
| 1350 | loop 5 |
| 1360 | loop 6 |
| 1370 | time |
| 1380 | viscosity |
| 1390 | slope |
| 1400 | log summary |
| 1410 | % thrombus |
| 1420 | aspiration |
| 1430 | infusion |
| 1450 | thrombus |
| 2000 | System A |
| 2001 | system A procedure #1 |
| 2002 | system A procedure #2 |
| 2003 | system A procedure #3 |
| 2004 | system A procedure #n |
| 2020 | Database/Compiler |
| 2025 | Data Exchange |
| 2100 | System B |
| 2101 | system B procedure # 1 |
| 2102 | system B procedure #2 |
| 2103 | system B procedure #3 |
| 2104 | system B procedure #n |
| 2200 | System C |
| 2201 | system C procedure # 1 |
| 2202 | system C procedure #2 |
| 2203 | system C procedure #3 |
| 2204 | system C procedure #n |
| 2302 | Thrombectomy System |
| 2310 | patient vascular system |
| 2320 | data log file |
| 2340 | surgical suite |
| 2402 | processor |
| 2404 | main memory |
| 2406 | storage device |
| 2408 | analog/digital I/O |
| 2410 | motor drivers |
| 2412 | audio generator |
| 2414 | video generator |
| 2416 | software/firmware |

1. An apparatus comprising:
a first fluid conduit having a proximal end and a distal end, wherein, in use of the apparatus, the distal end of the first fluid conduit is fluidically coupled to a fluid reservoir, the fluid reservoir including a material attached to a wall thereof; and
a reciprocating surface, wherein the reciprocating surface displaces fluid within the first fluid conduit, and wherein the reciprocating surface is operable to create an oscillating flow of a fluid within the first fluid conduit at a frequency less than 19,000 Hz, the oscillating flow within the first fluid conduit creating an oscillating motion of the material, thereby attriting the material.

2. The apparatus of claim 1 including:
a transducer fluidically coupled to the fluid conduit; and
a controller operable to receive input data from the transducer and vary the motion of the reciprocating surface.

3. The apparatus of claim 2 wherein varying the motion of the reciprocating surface comprises varying the speed of the motion of the reciprocating surface.

4. The apparatus of claim 2 wherein varying the motion comprises varying the distance of the motion.

5. The apparatus of claim 2 including a pump fluidically coupled to the first fluid conduit.

6. The apparatus of claim 5 including:
a second fluid conduit; and
a second pump fluidically coupled to the second fluid conduit.

7. The apparatus of claim 6 wherein:
the second fluid conduit is at a first location with respect to the first fluid conduit at a first time; and
the second fluid conduit is at a second location with respect to the first fluid conduit at a second time.

8. The apparatus of claim 2 wherein the distance between the proximal end of first fluid conduit and the distal end of the first fluid conduit is a first length at the first time; and
the distance between the proximal end of first fluid conduit and the distal end of the first fluid conduit is a second length at a second time.

9. The apparatus of claim 1 wherein the reciprocating surface comprises a face of a piston.

10. The apparatus of claim 1 wherein the reciprocating surface comprises a diaphragm.

11. The apparatus of claim 1 wherein the reciprocating surface comprises a sonic transducer.

12. The apparatus of claim 2 wherein the transducer is operable to measure pressure.

13. The apparatus of claim 2 wherein the transducer is operable to measure frequency.

14. The apparatus of claim 2 wherein the controller is operable to measure a viscosity of the fluid within the fluid conduit.

15. An apparatus comprising a fluid conduit, the fluid conduit having a proximal end and a distal end, the fluid conduit comprising:
a first lumen operable to transport a pressurized liquid from a proximal end thereof to a distal end thereof;
wherein, in use of the apparatus, the distal end of the fluid conduit is fluidically coupled to a fluid reservoir, and at least a portion of the pressurized liquid is discharged into the fluid reservoir in a radial direction, thereby generating a reaction force exerted upon the fluid conduit in a direction opposite to the radial direction, the fluid conduit thereby being forced into contact with a wall of the fluid reservoir.

16. The apparatus of claim 15 wherein the conduit comprises a second lumen, wherein the distal end of the first lumen is a distance, d, from a distal end of the second lumen, wherein in use of the apparatus, the distance, d, has a first value at a first time and a second value at a second time.

17. An apparatus comprising:
a) a fluid conduit having a proximal end and a distal end, wherein, in use of the apparatus, the distal end of the fluid conduit is fluidically coupled to a fluid reservoir, the fluid reservoir containing a fluid and including a material attached to a wall thereof, the material having different physical properties from the fluid;
b) a pumping system operable to generate a differential pressure between the proximal end and the distal end of the conduit, the pumping system having a setpoint and operable to create a flow of the fluid within the conduit;
c) means for attriting the material, the means having a setpoint;
d) a transducer operable to measure an amount of the material contained within the conduit;
e) a system controller, wherein the system controller:
(i) is operable to cause the means to attrite the material, receive measurement data from the transducer, adjust the setpoint of the pumping system, and adjust the setpoint of the means;
(ii) executes a first experiment comprising a combination of a first setpoint of the pumping system and a first setpoint of the means;
(iii) receives a first value of the measurement data based on the first experiment;
(iv) correlates, using the first value of the measurement data, the first experiment to the first measurement of the amount of the material contained within the catheter.

18. A method comprising:
(i) performing a first thrombectomy procedure using a thrombectomy apparatus, wherein the thrombectomy apparatus includes setpoint-controlled means to attrite thrombus, setpoint-controlled means to aspirate a fluid within a catheter, and means to measure thrombus within the catheter, wherein the first thrombectomy procedure comprises operating the thrombectomy apparatus at successive setpoints, wherein for each successive set point, an amount of thrombus within the catheter is measured and correlated to the respective setpoint, and wherein some of the setpoints positively correlate to the amount of thrombus within the catheter; and
(ii) performing a second thrombectomy procedure using the thrombectomy apparatus, wherein at least some of the setpoints that positively correlate to the amount of thrombus within the catheter are used to as setpoints for the setpoint-controlled means to attrite thrombus and the setpoint-controlled means to aspirate a fluid within a catheter.
